(19) United States
(12) Patent Application Publication
(10) Pub. No.: US 2022/0133802 A1
(43) Pub. Date: May 5, 2022

(54) FUSION POLYPEPTIDE FOR IMMUNOTHERAPY

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Michel Sadelain, New York, NY (US); Mohamad Hamieh, New York, NY (US); Anton Dobrin, New York, NY (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 17/578,989

(22) Filed: Jan. 19, 2022

Related U.S. Application Data
(63) Continuation of application No. PCT/US2020/042753, filed on Jul. 20, 2020.
(60) Provisional application No. 62/876,338, filed on Jul. 19, 2019.

(51) Int. Cl. A61K 35/17; C07K 14/725; A61P 35/00; C07K 14/705

(52) U.S. Cl. CPC A61K 35/17; C07K 14/7051; C07K 14/70532; C07K 14/70521; A61P 35/00

(57) ABSTRACT

The presently disclosed subject matter provides methods and compositions for enhancing immune responses toward tumor and pathogen antigens. It relates to fusion polypeptide that can be expressed in cells (e.g., immunoresponsive cells comprising an antigen-recognizing receptor) to improve the activity and/or efficiency of the cells. In certain embodiments, the fusion polypeptide comprises an extracellular domain and a transmembrane domain of a co-stimulatory ligand, and an intracellular domain of a co-stimulatory molecule.

### CROSS-REFERENCE TO RELATED APPLICATIONS

This application is a Continuation of International Patent Application No. PCT/US20/42753, filed Jul. 20, 2020, which claims priority to U.S. Provisional Patent Application Ser. No. 62/876,338, filed Jul. 19, 2019, the content of each of which is incorporated by reference in its entirety, and to each of which priority is claimed.

### SEQUENCE LISTING

The specification further incorporates by reference the Sequence Listing submitted herewith via EFS on Jan. 19, 2022. Pursuant to 37 C.F.R. § 1.52(e)(5), the Sequence Listing text file, identified as 0884860130SL.txt, is 126,063 bytes and was created on Jan. 19, 2022. The Sequence Listing electronically filed herewith, does not extend beyond the scope of the specification and thus does not contain new matter.

### 1. INTRODUCTION

The presently disclosed subject matter provides fusion polypeptides that can improve the activity and efficacy for immunotherapy.

### 2. BACKGROUND OF THE INVENTION

Cell-based immunotherapy is a therapy with curative potential for the treatment of cancer. T cells and other immune cells may be modified to target tumor antigens through the introduction of genetic material coding for natural or modified T cell receptors (TCR) or synthetic receptors for antigen, termed Chimeric Antigen Receptors (CARs), specific to selected antigens. Patient-engineered CAR T cells have demonstrated remarkable efficacy against a range of liquid and solid malignancies. However, treatment failure and relapses occur in a large fraction of patients. Therefore, there remain needs of improved immunotherapy.

### 3. SUMMARY OF THE INVENTION

The presently disclosed subject matter provides fusion polypeptides that can enhance the activity and efficacy of immunotherapy (e.g., T-cell immunotherapy). In certain embodiments, the fusion polypeptide comprises: a) an extracellular domain and a transmembrane domain of a co-stimulatory ligand, and b) an intracellular domain of a first co-stimulatory molecule.

In certain embodiments, the co-stimulatory ligand is selected from the group consisting of tumor necrosis factor (TNF) family members, and immunoglobulin (Ig) superfamily members, and combinations thereof. In certain embodiments, the TNF family member is selected from the group consisting of 4-1BBL, OX40L, CD70, GITRL, CD40L, and combinations thereof. In certain embodiments, the Ig superfamily member is selected from the group consisting of CD80, CD86, ICOSLG, and combinations thereof. In certain embodiments, the co-stimulatory ligand is CD80. In certain embodiments, the extracellular domain of CD80 comprise an amino acid sequence that is at least about 85% homologous or identical to the amino acid sequence set forth in SEQ ID NO: 1. In certain embodiments, the extracellular domain of CD80 comprises or consists of the amino acid sequence set forth in SEQ ID NO: 1 or a functional fragment thereof. In certain embodiments, the extracellular domain of CD80 comprises or consists of the amino acid sequence set forth in SEQ ID NO: 1. In certain embodiments, the transmembrane domain of CD80 comprise an amino acid sequence that is at least about 85% homologous or identical to the amino acid sequence set forth in SEQ ID NO: 2. In certain embodiments, the transmembrane domain of CD80 comprises or consists of the amino acid sequence set forth in SEQ ID NO: 2 or a fragment thereof. In certain embodiments, the transmembrane domain of CD80 comprises or consists of the amino acid sequence set forth in SEQ ID NO: 2.

In certain embodiments, the first co-stimulatory molecule is selected from the group consisting of CD28, 4-1BB, OX40, ICOS, DAP-10, CD27, CD40, NKG2D, CD2, and combinations thereof. In certain embodiments, the first co-stimulatory molecule is 4-1BB. In certain embodiments, the intracellular domain of 4-1BB comprises an amino acid sequence that is at least about 85% homologous or identical to the amino acid sequence set forth in SEQ ID NO: 3. In certain embodiments, the intracellular domain of 4-1BB comprises or consists of the amino acid sequence set forth in SEQ ID NO: 3 or a functional fragment thereof. In certain embodiments, the intracellular domain of 4-1BB comprises or consists of the amino acid sequence set forth in SEQ ID NO: 3.

In certain embodiments, the co-stimulatory ligand is CD80 and the first co-stimulatory molecule is selected from the group consisting of CD28, 4-1BB, OX40, ICOS, DAP-10, CD27, CD40, NKG2D, CD2, and combinations thereof.

In certain embodiments, the co-stimulatory ligand is CD80 and the first co-stimulatory molecule is 4-1BB. In certain embodiments, the fusion polypeptide comprises an amino acid sequence that is at least about 85% homologous or identical to the amino acid sequence set forth in SEQ ID NO: 4. In certain embodiments, the fusion polypeptide comprises or consists of the amino acid sequence set forth in SEQ ID NO: 4.

In certain embodiments, the fusion polypeptide further comprises an intracellular domain of a second co-stimulatory molecule. In certain embodiments, the second co-stimulatory molecule is selected from the group consisting of CD28, 4-1BB, OX40, ICOS, DAP-10, CD27, CD40, NKG2D, CD2, and combinations thereof. In certain embodiments, the second co-stimulatory molecule is CD28. In certain embodiments, the first co-stimulatory molecule is 4-1BB and the second co-stimulatory molecule is CD28. In certain embodiments, the intracellular domain of CD28 comprises an amino acid sequence that is at least about 85% homologous or identical to the amino acid sequence set forth in SEQ ID NO: 5. In certain embodiments, the intracellular domain of CD28 comprises or consists of the amino acid sequence set forth in SEQ ID NO: 5 or a functional fragment thereof. In certain embodiments, the intracellular domain of CD28 comprises or consists of the amino acid sequence set forth in SEQ ID NO: 5.

In certain embodiments, the co-stimulatory ligand is CD80, the first co-stimulatory molecule is 4-1BB, and the second co-stimulatory molecule is CD28. In certain embodiments, the fusion polypeptide comprises an amino acid sequence that is at least about 85% homologous or identical to the amino acid sequence set forth in SEQ ID NO: 6. In certain embodiments, the fusion polypeptide comprises or consists of the amino acid sequence set forth in SEQ ID NO: 6.

In certain embodiments, the fusion polypeptide is capable of stimulating a cell comprising an antigen-recognizing receptor. In certain embodiments, the fusion polypeptide is capable of enhancing the activity of a cell comprising an antigen-recognizing receptor. In certain embodiments, the activity includes cytotoxicity, cell proliferation, cell persistence, and combinations thereof.

In certain embodiments, the fusion polypeptide further comprises a signaling domain of a cytokine receptor. In certain embodiments, the cytokine receptor is selected from the group consisting of CD121a, CDw121b, IL-18Ra, IL18Rb, CD122, CD25, CD132, CD124, CD213a13, CD127, IL-9R, IL15Ra, CDw125, CDw131, CD126, CD130, IL11Ra, Cd114, CD212, CD4, CDw217, CD118, and CDw119.

The presently disclosed subject matter further provides nucleic acid molecules encoding a fusion polypeptide disclosed herein and vectors comprising the nucleic acid molecules.

The presently disclosed subject matter also provides cells comprising the fusion polypeptide, the nucleic acid molecule, or the vector disclosed herein.

In certain embodiments, the cell further comprises an antigen-recognizing receptor. In certain embodiments, the antigen recognizing receptor is selected from the group consisting of a chimeric antigen receptor (CAR), a T cell receptor (TCR), and a TCR like fusion molecule.

In certain embodiments, the antigen is a tumor antigen or a pathogen antigen. In certain embodiments, the antigen is a tumor antigen. In certain embodiments, the tumor antigen is selected from the group consisting of CD19, carbonic anhydrase IX (CAlX), carcinoembryonic antigen (CEA), CD8, CD7, CD10, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinase Erb-B2, Erb-B3, Erb-B4, folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-VIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A, and ERBB. In certain embodiments, the tumor antigen is CD19.

In certain embodiments, the antigen-recognizing receptor is exogenous or endogenous. In certain embodiments, the antigen recognizing receptor is recombinantly expressed. In certain embodiments, the antigen-recognizing receptor is expressed from a vector.

In certain embodiments, the cell is a cell of the lymphoid lineage. In certain embodiments, the cell is a cell of the myeloid lineage. In certain embodiments, the cell of the lymphoid lineage is selected from the group consisting of T cells, Natural Killer (NK) cells, B cells, dendritic cells, and, stem cell from which lymphoid cells may be differentiated. In certain embodiments, the cell is a T cell. In certain embodiments, the T cell is derived from an induced pluripotent stem cell. In certain embodiments, the T cell is a CD8⁺ T cell. In certain embodiments, the CD8⁺ T cell is CD4 independent. In certain embodiments, the T cell is selected from the group consisting of a cytotoxic T lymphocyte (CTL), a regulatory T cell, a γδ T cell, a tumor-infiltrating lymphocyte (TIL), and a Natural Killer T (NKT) cell. In certain embodiments, the fusion polypeptide and the antigen-recognizing receptor are integrated at a locus within the genome of the T cell. In certain embodiments, the locus is selected from the group consisting of a TRAC locus, a TRBC locus, a TRDC locus, and a TRGC locus. In certain embodiments, the locus is a TRAC locus or a TRBC locus. In certain embodiments, the locus is a TRAC locus. In certain embodiments, the antigen-recognizing receptor is a chimeric antigen receptor (CAR). In certain embodiments, the expressions of the antigen-recognizing receptor and the fusion polypeptides are under the control of an endogenous promoter. In certain embodiments, the endogenous promoter is selected from the group consisting of an endogenous TRAC promoter, an endogenous TRBC promoter, an endogenous TRDC promoter, and an endogenous TRGC promoter. In certain embodiments, the endogenous promoter is a TRAC promoter.

In certain embodiments, the fusion polypeptide and the antigen-recognizing receptor are integrated within a locus encoding an immune inhibitory molecule. In certain embodiments, the immune inhibitory molecule is selected the group consisting of CTLA-4, PD-1, LAG3, BTLA, B7-1, B7-H1, B7-H3, B7-H4, TIM3, SHP-1, SHP-2, TIGIT, CD160, and LAIR1.

In certain embodiments, the cell is autologous. In certain embodiments, the cell is allogeneic.

The presently disclosed subject matter provides compositions comprising a cell disclosed herein. In certain embodiments, the composition is a pharmaceutical composition that further comprises a pharmaceutically acceptable excipient. In certain embodiments, the pharmaceutical composition is for treating and/or preventing a neoplasm, an autoimmune disease, and/or an infectious disease. In certain embodiments, the neoplasm is cancer.

In certain embodiments, the composition further comprises a regulator that is capable of regulating or modulating expression and/or activity of the fusion polypeptide.

In certain embodiments, the regulator is selected from the group consisting of promoters that are capable of controlling the expression of the fusion polypeptide, molecules that are capable of regulating or modulating expression and/or activity of the co-stimulatory ligand, and molecules that are capable of regulating or modulating expression and activity of the co-stimulatory molecule.

In certain embodiments, the molecules that are capable of regulating or modulating expression and/or activity of the co-stimulatory ligand are selected from the group consisting of antibodies that bind to the co-stimulatory ligand, and fusion proteins that bind to the co-stimulatory ligand and regulate or modulate the expression and/or activity of the co-stimulatory ligand.

In certain embodiments, the regulator is an anti-CD80 antibody and the fusion polypeptide comprises an extracellular domain and a transmembrane domain of CD80.

In certain embodiments, the regulator is a fusion protein that binds to CD80 and modulates the activity of CD80 and the fusion polypeptide comprises an extracellular domain and a transmembrane domain of CD80. In certain embodiments, the fusion protein comprises a CTLA-4 fragment that binds to CD80. In certain embodiments, the CTLA-4 fragment that binds to CD80 is Abatacept or belatacept.

In certain embodiments, the molecules that are capable of regulating or modulating expression and/or activity of the co-stimulatory molecule are selected from the group consisting of antibodies that bind to the co-stimulatory molecule, and fusion proteins that bind to the co-stimulatory molecule and regulate or modulate the expression and/or activity of the co-stimulatory molecule.

The presently disclosed subject matter further provides compositions comprising: a) a fusion polypeptide disclosed herein; and b) an antigen-recognizing receptor that binds to an antigen. In certain embodiments, the fusion polypeptide is operably linked to a first promoter. In certain embodiments, the antigen-recognizing receptor is operably linked to a second promoter.

In addition, the presently disclosed subject matter further provides nucleic acid compositions comprising: a) a first polynucleotide encoding a fusion polypeptide disclosed herein; and b) a second polynucleotide encoding an antigen-recognizing receptor that binds to an antigen. In certain embodiments, the nucleic acid composition further comprises a first promoter that is operably linked to the fusion polypeptide. In certain embodiments, the nucleic acid composition further comprises a second promoter that is operably linked to the antigen-recognizing receptor.

In certain embodiments, one or both of the first and second promoters are endogenous or exogenous. In certain embodiments, the exogenous promoter is selected from the group consisting of an elongation factor (EF)-1 promoter, a cytomegalovirus immediate-early promoter (CMV) promoter, a simian virus 40 early promoter (SV40) promoter, a phosphoglycerate kinase (PGK) promoter, and a metallothionein promoter. In certain embodiments, one or both of the first and second promoters are inducible promoters. In certain embodiment, the inducible promoter is selected from a NFAT transcriptional response element (TRE) promoter, a CD69 promoter, a CD25 promoter, and an IL-2 promoter.

The presently disclosed subject matter further provides cells comprising the compositions disclosed herein or the nucleic acid compositions disclosed herein. In certain embodiments, the composition or the nucleic acid composition is integrated at a locus within the genome of the T cell. In certain embodiments, the locus is selected from the group consisting of a TRAC locus, a TRBC locus, a TRDC locus, and a TRGC locus. In certain embodiments, the locus is a TRAC locus or a TRBC locus. In certain embodiments, the composition or the nucleic acid composition is integrated within a locus encoding an immune inhibitory molecule. In certain embodiments, the immune inhibitory molecule is selected from the group consisting of CTLA-4, PD-1, LAG3, BTLA, B7-1, B7-H1, B7-H3, B7-H4, TIM3, SHP-1, SHP-2, TIGIT, CD160, and LAIR1.

In certain embodiments, the cells, the compositions, or the nucleic acid compositions disclosed herein are for use in reducing tumor burden, treating and/or preventing a neoplasm, treating and/or preventing an autoimmune disease, treating and/or preventing a pathogen infection (e.g., an infectious disease), and/or treating a subject having a relapse of a neoplasm.

The presently disclosed subject matter further provides methods for producing a cell. In certain embodiments, the method comprises introducing into a cell a nucleic acid molecule, a nucleic acid composition, or a vector disclosed herein.

The presently disclosed subject matter provides kits comprising a nucleic acid molecule, a vector, a cell, a composition, or a nucleic acid composition disclosed herein. In certain embodiments, the kit further comprises written instructions for treating and/or preventing a neoplasm, a pathogen infection (e.g., an infectious disease), and/or an autoimmune disease.

The presently disclosed subject matter provides methods of reducing tumor burden, treating a subject having a relapse of a neoplasm (e.g., a cancer), treating and/or preventing a neoplasm (e.g., a cancer), treating and/or preventing a pathogen infection (e.g., an infectious disease), and/or treating and/or preventing an autoimmune disease, in a subject. In certain embodiments, the method comprises administering to the subject an effective amount of the cells, a composition, or a nucleic acid composition disclosed herein. In certain embodiments, the method reduces the number of tumor cells, reduces tumor size, and/or eradicates the tumor in the subject. In certain embodiments, the neoplasm or tumor is selected from the group consisting of blood cancers and solid tumors. In certain embodiments, the neoplasm or tumor is a solid tumor. In certain embodiments, the blood cancer is selected from the group consisting of B cell leukemia, multiple myeloma, acute lymphoblastic leukemia (ALL), chronic lymphocytic leukemia, and non-Hodgkin's lymphoma.

In certain embodiments, the autoimmune disease and infectious disease are selected from the group consisting of rheumatoid arthritis (RA), Type I diabetes, systemic lupus erythematosus (SLE), inflammatory bowel disease, ulcerative colitis, psoriasis, psoriatic arthritis, scleroderma, autoimmune thyroid disease, Grave's disease, Crohn's disease, multiple sclerosis, systemic sclerosis, asthma, organ transplant rejection, a disease or condition associated with transplant, Takayasu arteritis, giant-cell arteritis, Kawasaki disease, polyarteritis nodosa, Behcet's syndrome, Wegener's granulomatosis, ANCA-vasculitides, Churg-Strauss syndrome, microscopic polyangiitis, vasculitis of connective tissue diseases, Hennoch-Schonlein purpura, cryoglobulinemic vasculitis, cutaneous leukocytoclastic angiitis, Sarcoidosis, Cogan's syndrome, Wiskott-Aldrich Syndrome, primary angiitis of the CNS, thromboangiitis obliterans, paraneoplastic arteritis, myelodysplastic syndrome, erythema elevatum diutinum, amyloidosis, autoimmune myositis, Guillain-Barre Syndrome, histiocytosis, atopic dermatitis, pulmonary fibrosis, glomerulonephritis, Whipple's disease, Still's disease, Sjogren's syndrome, osteomyelofibrosis, chronic inflammatory demyelinating polyneuropathy, Kimura's disease, systemic sclerosis, chronic periaortitis, chronic prostatitis, idiopathic pulmonary fibrosis, chronic granulomatous disease, idiopathic, bleomycin-induced lung inflammation, cytarabine-induced lung inflammation, autoimmune thrombocytopenia, autoimmune neutropenia, autoimmune hemolytic anemia, autoimmune lymphocytopenia, chronic autoimmune thyroiditis, autoimmune hepatitis, Hashimoto's thyroiditis, atopic thyroiditis, Graves disease, autoimmune polyglandular syndrome, autoimmune Addison syndrome, and myasthenia gravis.

In certain embodiments, the method further comprises administering to the subject a regulator that is capable of regulating or modulating expression and/or activity of the fusion polypeptide. In certain embodiments, the regulator is selected from the group consisting of promoters that are capable of controlling the expression of the fusion polypeptide, molecules that are capable of regulating or modulating expression and/or activity of the co-stimulatory ligand, and molecules that are capable of regulating or modulating expression and/or activity of the co-stimulatory molecule. In certain embodiments, the molecules that are capable of regulating or modulating expression and/or activity of the co-stimulatory ligand are selected from the group consisting of antibodies that bind to the co-stimulatory ligand, and fusion proteins that bind to the co-stimulatory ligand and regulate or modulate the expression, activity of the co-stimulatory ligand. In certain embodiments, the regulator is an anti-CD80 antibody and the fusion polypeptide comprises an extracellular domain and a transmembrane domain of CD80. In certain embodiments, the regulator is a fusion protein that binds to CD80 and modulates the activity of CD80 and the fusion polypeptide comprises an extracellular domain and a transmembrane domain of CD80. In certain embodiments, the fusion protein is a CTLA4 fragment that binds to CD80. In certain embodiments, the CTLA4 fragment that binds to CD80 is Abatacept or belatacept.

In certain embodiments, the molecules that are capable of regulating or modulating expression and/or activity of the co-stimulatory molecule are selected from the group consisting of antibodies that bind to the co-stimulatory molecule, and fusion proteins that bind to the co-stimulatory molecule and regulate or modulate the expression and/or activity of the co-stimulatory molecule.

In certain embodiments, the regulator is capable of depleting the cell. In certain embodiments, the regulator is capable of lessening or eliminating one or more side effects associated with administration of the cell. In certain embodiments, the one or more side effects are selected from the group consisting of off-tumor target effects, cytokine release syndrome, neurotoxicity, and combinations thereof.

In certain embodiments, the method further comprises administering to the subject a checkpoint immune blockade agent. In certain embodiments, the checkpoint immune blockade agent is selected from the group consisting of anti-PD-L1 antibodies, anti-CTLA-4 antibodies, anti-PD-1 antibodies, anti-LAG3 antibodies, anti-B7-H3 antibodies, anti-TIM3 antibodies, and combinations thereof. In certain embodiments, the checkpoint immune blockade agent is an anti-PD-1 antibody.

### 4. BRIEF DESCRIPTION OF THE FIGURES

The following Detailed Description, given by way of example, but not intended to limit the presently disclosed subject matter to specific embodiments described, may be understood in conjunction with the accompanying drawings.

FIG. 1 depicts a cell in accordance with certain embodiments of the presently disclosed subject matter.

FIGS. 2A-2H depict Universal CD80-4-1BB fusion polypeptide to boost antitumor T cell response. FIG. 2A shows FACS profile depicting the expression of 1928z CAR with (right) or without (left) the CD80-4-1BB fusion polypeptide. T cells are transduced with gamma retroviral vector to stably express 1928z and the CD80/4-1BB fusion polypeptide. FIG. 2B depicts the Kaplan-Meier survival analysis of mice bearing NALM6 CD19 leukemia cells treated with 10⁵ 19-28-z CAR T cells co-expressing an exogenous CD80 molecule, an exogenous 4-1BBL molecule, or the CD80/4-1BB fusion polypeptide. FIG. 2C depicts the FACS profile depicting the expression of 1928z-1xx CAR T cells with (right) or without (left) the CD80/4-1BB fusion polypeptide. 1928z-1xx and the CD80/4-1BB fusion polypeptide were targeted into TRAC locus and were expressed under the control of the endogenous TRAC promoter. FIG. 2D depicts the Kaplan-Meier survival analysis of mice bearing NALM6 CD19 leukemia cells treated with 2×10⁴ 19-28-z-1xx CART cells with or without co-expression of the CD80/4-1BB fusion polypeptide. FIG. 2E depicts the FACS profile depicting the expression of 19-HIT with (middle and right) or without (left) the CD80/4-1BB fusion polypeptide. FIG. 2E Left and FIG. 2E Middle are directed to 19-HIT and the CD80/4-1BB fusion polypeptide targeted into TRAC locus and expressed under the control of the endogenous TRAC promoter. FIG. 2E Right is directed to 19-HIT T cells transduced with SFG gamma-retroviral vector to express high levels of the CD80/4-1BB fusion polypeptide. FIG. 2F depicts the Kaplan-Meier survival analysis of mice bearing NALM6 CD19 leukemia cells treated with 10⁵ 19-HIT T cells with or without the CD80/4-1BB fusion polypeptide. FIG. 2G depicts the FACS profile depicting the expression of NY-ESO-1 TCR with (right) or without (left) the CD80/4-1BB fusion polypeptide. Gamma retroviral vector encoding for NY-ESO-1 TCR or the CD80/4-1BB fusion polypeptide were used. FIG. 2H depicts the Kaplan-Meier survival analysis of mice bearing SK-MEL-23 melanoma cell line expressing HLAA2.1/NYESO-complex treated with 2×10⁶ NY-ESO-1 TCR T cells with or without the CD80/4-1BB fusion polypeptide. P values were determined by log-rank Mantel-Cox test.

FIGS. 3A and 3B depict the maintenance of CD80-4-1BB anti-tumor benefit in presence of PDL-1 inhibitory ligand. FIG. 3A depicts the Kaplan-Meier survival analysis of mice bearing NALM6 CD19 leukemia cells overexpressing PDL-1 molecule treated with 5×10⁴ 19-CAR T cells with or without the CD80/4-1BB fusion polypeptide. FIG. 3B depicts the Kaplan-Meier survival analysis of mice bearing NALM6 CD19 leukemia cells overexpressing PDL-1 molecule treated with 10⁵ HIT T cells with or without the CD80/4-1BB fusion polypeptide. P values were determined by log-rank Mantel-Cox test.

FIGS. 4A and 4B depict that CD80-4-1BB expression level governs anti-tumor response efficiency. FIG. 4A depicts the FACS profile depicting the expression of the CD80/4-1BB fusion polypeptide under the control of endogenous TRAC locus or exogenous EF1a or PGK promoters targeted into the TRBC locus. In all conditions, 1928z-1xx was expressed under the control of endogenous TRAC promoter. T cells were bead-sorted after transduction based on CD80 expression using an anti-CD80 antibody. Mean Fluorescence Intensity of CD80 was shown. FIG. 4B shows the total flux signal of NALM6 tumor cells expressing Firefly Luciferase15 days post CART cell injection. P values were determined Mann-Whitney t-test.

FIG. 5 depicts the Kaplan-Meier survival analysis of mice bearing NALM6 CD19 leukemia cells treated with 2.5×10⁴ 1928 z-1xx CAR T cells with the CD80/4-1BB fusion polypeptide. Endogenous 4-1BB-gene knockout was obtained by electroporation of protein CAS9 and specific gRNA. Disruption of 4-1BB was achieved in 68% of infused CAR T cells. TRBC gRNA was used as a control. P values were determined by log-rank Mantel-Cox test.

FIG. 6 depicts the Kaplan-Meier survival analysis of mice bearing NALM6 CD19 leukemia cells treated with 2.5×10⁴ CAR T cells. Endogenous PD-1-gene knockout obtained by electroporation of protein CAS9 and specific gRNA. Disruption of PD-1 is achieved in 55% of infused CAR T cells. TRBC gRNA is used as a control. P values were determined by log-rank Mantel-Cox test.

FIGS. 7A and 7B depict the Kaplan-Meier survival analysis of mice bearing NALM6 CD19 leukemia cells treated with 8×10⁵ 1928 z CAR CD8⁺ T cells with the CD80/4-1BB fusion polypeptide (FIG. 7A) or 4×10⁵ 1928 z CAR CD8⁺ T cells with the CD80/4-1BB fusion polypeptide (FIG. 7B). P values were determined by log-rank Mantel-Cox test.

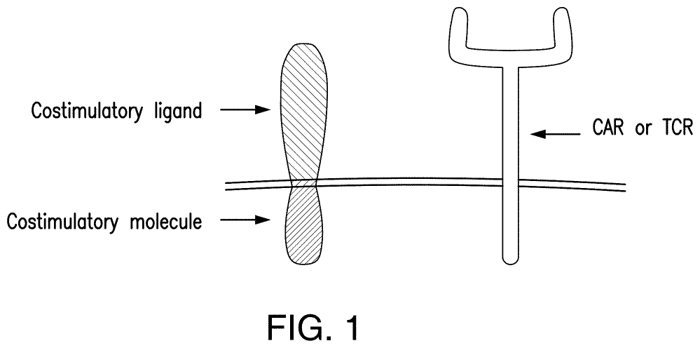

### 5. DETAILED DESCRIPTION OF THE INVENTION

The presently disclosed subject matter provides fusion polypeptides that are capable of enhancing the activity and/or efficacy of immunotherapy (e.g., T cell immunotherapy). The fusion polypeptide can enhance the activity and/or efficacy of cells (e.g., T cells or NK cells) comprising an antigen-recognizing receptor (e.g., a CAR, a TCR, or a TCR like fusion molecule). The presently disclosed subject matter also provides methods of using such fusion polypeptide for inducing and/or enhancing an immune response of a cell to a target antigen, and/or treating and/or preventing neoplasms or other diseases/disorders (e.g., autoimmune diseases and infectious diseases), e.g., where an increase in an antigen-specific immune response is desired. The presently disclosed subject matter is based, at least in part, on the discovery that a fusion polypeptide disclosed herein can enhance the activity (e.g., cytotoxicity) of a cell comprising an antigen-recognizing receptor (e.g., a CAR, a TCR, or a TCR like fusion molecule).

Non-limiting embodiments of the present disclosure are described by the present specification and Examples.

For purposes of clarity of disclosure and not by way of limitation, the detailed description is divided into the following subsections:

5.1. Definitions;

5.2. Fusion Polypeptides;

5.3. Cells;

5.4. Antigen-Recognizing Receptors;

5.5. Compositions and Vectors;

5.6. Polypeptides and Analogs;

5.7. Administration;

5.8. Formulations;

5.9. Methods of Treatment; and

5.10. Kits.

### 5.1. Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

As used herein, a “co-stimulatory molecule” refer to a cell surface molecule other than an antigen receptor or its ligand that can provide an efficient response of lymphocytes to an antigen. In certain embodiments, a co-stimulatory molecule can provide optimal lymphocyte activation.

As used herein, a “co-stimulatory ligand” refers to a molecule that upon binding to its receptor (e.g., a co-stimulatory molecule) produces a co-stimulatory response, e.g., an intracellular response that effects the stimulation provided when an antigen-recognizing receptor (e.g., a chimeric antigen receptor (CAR)) binds to its target antigen.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40 and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of activating an immune or immunoresponsive cell (e.g., a T-cell) in response to its binding to an antigen.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., J. Nucl. Med. 24:316-325 (1983). As used herein, antibodies include whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242).

As used herein, the term “single-chain variable fragment” or “scFv” is a fusion protein of the variable regions of the heavy (V_{H}) and light chains (V_{L}) of an immunoglobulin covalently linked to form a V_{H}::V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Biol Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after formation of reversible complexes. Methods for calculating the affinity of an antibody for an antigen are known in the art, including, but not limited to, various antigen-binding experiments, e.g., functional assays (e.g., flow cytometry assay).

The term “chimeric antigen receptor” or “CAR” as used herein refers to a molecule comprising an extracellular antigen-binding domain that is fused to an intracellular signaling domain that is capable of activating or stimulating an immune or immunoresponsive cell, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

As used herein, the term “nucleic acid molecules” include any nucleic acid molecule that encodes a polypeptide of interest or a fragment thereof. Such nucleic acid molecules need not be 100% homologous or identical with an endogenous nucleic acid sequence, but may exhibit substantial identity. Polynucleotides having “substantial identity” or “substantial homology” to an endogenous sequence are typically capable of hybridizing with at least one strand of a double-stranded nucleic acid molecule. By “hybridize” is meant a pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, e.g., less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, e.g., at least about 50% formamide. Stringent temperature conditions will ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In certain embodiments, hybridization will occur at 30° C. in 750 mM NaCl, 75 mM trisodium citrate, and 1% SDS. In certain embodiments, hybridization will occur at 37° C. in 500 mM NaCl, 50 mM trisodium citrate, 1% SDS, 35% formamide, and 100 μg/ml denatured salmon sperm DNA (ssDNA). In certain embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps can be less than about 30 mM NaCl and 3 mM trisodium citrate, e.g., less than about 15 mM NaCl and 1.5 mM trisodium citrate. Stringent temperature conditions for the wash steps will ordinarily include a temperature of at least about 25° C., of at least about 42° C., or of at least about 68° C. In certain embodiments, wash steps will occur at 25° C. in 30 mM NaCl, 3 mM trisodium citrate, and 0.1% SDS. In certain embodiments, wash steps will occur at 42° C. in 15 mM NaCl, 1.5 mM trisodium citrate, and 0.1% SDS. In certain embodiments, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Rogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” or “substantially homologous” is meant a polypeptide or nucleic acid molecule exhibiting at least about 50% homologous or identical to a reference amino acid sequence (for example, any one of the amino acid sequences described herein) or nucleic acid sequence (for example, any one of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

By “analog” is meant a structurally related polypeptide or nucleic acid molecule having the function of a reference polypeptide or nucleic acid molecule.

The term “ligand” as used herein refers to a molecule that binds to a receptor. In certain embodiments, the ligand binds to a receptor on another cell, allowing for cell-to-cell recognition and/or interaction.

The term “constitutive expression” or “constitutively expressed” as used herein refers to expression or expressed under all physiological conditions.

By “disease” is meant any condition, disease or disorder that damages or interferes with the normal function of a cell, tissue, or organ, e.g., neoplasm, and pathogen infection of cell.

By “effective amount” is meant an amount sufficient to have a therapeutic effect. In certain embodiments, an “effective amount” is an amount sufficient to arrest, ameliorate, or inhibit the continued proliferation, growth, or metastasis (e.g., invasion, or migration) of a neoplasm.

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

“Linker”, as used herein, shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises a sequence set forth in GGGGSGGGGSGGGGS [SEQ ID NO: 7].

By “neoplasm” is meant a disease characterized by the pathological proliferation of a cell or tissue and its subsequent migration to or invasion of other tissues or organs. Neoplasm growth is typically uncontrolled and progressive, and occurs under conditions that would not elicit, or would cause cessation of, multiplication of normal cells. Neoplasm can affect a variety of cell types, tissues, or organs, including but not limited to an organ selected from bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasms include cancers, such as sarcomas, carcinomas, or plasmacytomas (malignant tumor of the plasma cells). In certain embodiments, the neoplasm is cancer.

By “receptor” is meant a polypeptide, or portion thereof, present on a cell membrane that selectively binds one or more ligand.

By “recognize” is meant selectively binds to a target. A T cell that recognizes a tumor can expresses a receptor (e.g., a TCR or CAR) that binds to a tumor antigen.

By “reference” or “control” is meant a standard of comparison. For example, the level of scFv-antigen binding by a cell expressing a CAR and an scFv may be compared to the level of scFv-antigen binding in a corresponding cell expressing CAR alone.

By “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

By “signal sequence” or “leader sequence” is meant a peptide sequence (e.g., 5, 10, 15, 20, 25 or 30 amino acids) present at the N-terminus of newly synthesized proteins that directs their entry to the secretory pathway. Exemplary leader sequences include, but is not limited to, a human IL-2 signal sequence (e.g., MYRMQLLSCIALSLALVTNS [SEQ ID NO: 8]), a mouse IL-2 signal sequence (e.g., MYSMQLASCVTLTLVLLVNS [SEQ ID NO: 9]); a human kappa leader sequence (e.g., METPAQLLFLLLLWLPDTTG [SEQ ID NO: 10]), a mouse kappa leader sequence (e.g., METDTLLLWVLLLWVPGSTG [SEQ ID NO: 11]); a human CD8 leader sequence (e.g., MALPVTALLLPLALLLHAARP [SEQ ID NO: 12]); a truncated human CD8 signal peptide (e.g., MALPVTALLLPLALLLHA [SEQ ID NO: 13]); a human albumin signal sequence (e.g., MKWVTFISLLFSSAYS [SEQ ID NO: 14]); and a human prolactin signal sequence (e.g., MDSKGSSQKGSRLLLLLVVSNLLLCQGVVS [SEQ ID NO: 15]).

By “soluble” is meant a polypeptide that is freely diffusible in an aqueous environment (e.g., not membrane bound).

By “specifically binds” is meant a polypeptide or fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein refers to an antigen (e.g., a polypeptide) that is uniquely or differentially expressed on a tumor cell compared to a normal or non-neoplastic cell. In certain embodiments, a tumor antigen includes any polypeptide expressed by a tumor that is capable of activating or inducing an immune response via an antigen recognizing receptor (e.g., CD19, MUC-16) or capable of suppressing an immune response via receptor-ligand binding (e.g., CD47, PD-L1/L2, B7.1/2).

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

As used herein, “a functional fragment” of a molecule or polypeptide includes a fragment of the molecule or polypeptide that retains at least about 80%, at least about 85%, at least about 90%, at least about 95%, or at least about 100% of the primary function of the molecule or polypeptide.

Other aspects of the presently disclosed subject matter are described in the following disclosure and are within the ambit of the presently disclosed subject matter.

### 5.2. Fusion Polypeptides

The presently disclosed subject matter provides fusion polypeptides that are capable of enhancing the activity and/or efficacy of a cell comprising an antigen-recognizing receptor (e.g., a CAR, a TCR, or a TCR like fusion molecule). In certain embodiments, the fusion polypeptide comprises: a) an extracellular domain and a transmembrane domain of a co-stimulatory ligand, and b) an intracellular domain of a first co-stimulatory molecule.

5.2.1. Co-Stimulatory Ligands

The co-stimulatory ligand can be selected from the group consisting of tumor necrosis factor (TNF) family members, immunoglobulin (Ig) superfamily members, and combinations thereof. The TNF family member can be selected from the group consisting of 4-1BBL, OX40L, CD70, GITRL, CD40L, and combinations thereof. The Ig superfamily member can be selected from the group consisting of CD80, CD86, ICOS ligand (ICOSLG (also known as “CD275”), and combinations thereof.

In certain embodiments, the co-stimulatory ligand is CD80. In certain embodiments, the co-stimulatory ligand is human CD80. In certain embodiments, the CD80 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_005182 (SEQ ID NO: 16) or a fragment thereof, and/or may optionally comprise up to one or up to two or up to three conservative amino acid substitutions. SEQ ID NO: 16 is provided below.

| [SEQ ID NO: 16] | |
|---|---|
| 1 | MGHTRRQGTS PSKCPYLNFF QLLVLAGLSH FCSGVIHVTK EVKEVATLSC GHNVSVEELA |
| 61 | QTRIYWQKEK KMVLTMMSGD MNIWPEYKNR TIFDITNNLS IVILALRPSD EGTYECVVLK |
| 121 | YEKDAFKREH LAEVTLSVKA DFPTPSISDF EIPTSNIRRI ICSTSGGFPE PHLSWLENGE |
| 181 | ELNAINTTVS QDPETELYAV SSKLDFNMTT NHSFMCLIKY GHLRVNQTFN WNTTKQEHFP |
| 241 | DNLLPSWAIT LISVNGIFVI CCLTYCFAPR CRERRRNERL RRESVRPV |

In certain embodiments, an extracellular domain of CD80 comprises or consists of an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 1 (or amino acids 1-242 of SEQ ID NO: 16). In certain embodiments, an extracellular domain of CD80 comprises or consists of the amino acid sequence set forth in SEQ ID NO: 1 or a functional fragment thereof. A functional fragment of SEQ ID NO: 1 can be a consecutive portion of SEQ ID NO: 1, which is at least about 50, at least about 75, at least about 100, at least about 125, at least about 150, at least about 175, or at least about 200, or at least about 220 amino acids in length. In certain embodiments, the functional fragment of SEQ ID NO: 1 retains at least about 80%, at least about 85%, at least about 90%, at least about 95%, or at least about 100% of the primary function of the extracellular domain of CD80. Non-limiting examples of the primary functions of the extracellular domain of CD80 include binding to/interacting with CD28, binding to/interacting with CTLA-4, binding to/interacting with PD-L1, and contributing to CD80 homodimerization. In certain embodiments, an extracellular domain of CD80 comprises or consists of the amino acid sequence set forth in SEQ ID NO: 1 (or amino acids 1-242 of SEQ ID NO: 16). SEQ ID NO: 1 is provided below.

[SEQ ID NO: 1]
MGHTRRQGTSPSKCPYLNFFQLLVLAGLSHFCSGVIHVTKEVKEVATLSC
GHNVSVEELAQTRIYWQKEKKMVLTMMSGDMNIWPEYKNRTIFDITNNLS
IVILALRPSDEGTYECVVLKYEKDAFKREHLAEVTLSVKADEPTPSISDF
EIPTSNIRRIICSTSGGFPEPHLSWLENGEELNAINTTVSQDPETELYAV
SSKLDFNMTTNHSFMCLIKYGHLRVNQTFNWNTTKQEHFPDN

In certain embodiments, a transmembrane domain of CD80 comprises or consists of an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 2 (or amino acids 243-263 of SEQ ID NO: 16). In certain embodiments, a transmembrane domain of CD80 comprises or consists of the amino acid sequence set forth in SEQ ID NO: 2 or a fragment thereof. A fragment of SEQ ID NO: 2 can be at least about 5, at least about 10, at least about 15, or at least about 20 amino acids in length. In certain embodiments, a transmembrane domain of CD80 comprises or consists of the amino acid sequence set forth in SEQ ID NO: 2 (or amino acids 243-263 of SEQ ID NO: 16). SEQ ID NO: 2 is provided below.

[SEQ ID NO: 2]
LLPSWAITLISVNGIFVICCL

In certain embodiments, the co-stimulatory ligand is 4-1BBL. In certain embodiments, the co-stimulatory ligand is human 4-1BBL. In certain embodiments, the 4-1BBL comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_003802.1 (SEQ ID NO: 17) or a fragment thereof, and/or may optionally comprise up to one or up to two or up to three conservative amino acid substitutions. SEQ ID NO: 17 is provided below.

| [SEQ ID NO: 17] | |
|---|---|
| 1 | MEYASDASLD PEAPWPPAPR ARACRVLPWA LVAGLLLLLL LAAACAVFLA CPWAVSGARA |
| 61 | SPGSAASPRL REGPELSPDD PAGLLDLRQG MFAQLVAQNV LLIDGPLSWY SDPGLAGVSL |
| 121 | TGGLSYKEDT KELVVAKAGV YYVFFQLELR RVVAGEGSGS VSLALHLQPL RSAAGAAALA |
| 181 | LTVDLPPASS EARNSAFGFQ GRLLHLSAGQ RLGVHLHTEA RARHAWQLTQ GATVLGLFRV |
| 241 | TPEIPAGLPS PRSE |

In certain embodiments, the co-stimulatory ligand is OX40L. In certain embodiments, the co-stimulatory ligand is human OX40L. In certain embodiments, the OX40L comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_003317 (SEQ ID NO: 18) or a fragment thereof, and/or may optionally comprise up to one or up to two or up to three conservative amino acid substitutions. SEQ ID NO: 18 is provided below.

| [SEQ ID NO: 18] | |
|---|---|
| 1 | MERVQPLEEN VGNAARPRFE RNKLLLVASV IQGLGLLLCF TYICLHFSAL QVSHRYPRIQ |
| 61 | SIKVQFTEYK KEKGFILTSQ KEDEIMKVQN NSVIINCDGF YLISLKGYFS QEVNISLHYQ |
| 121 | KDEEPLFQLK KVRSVNSLMV ASLTYKDKVY LNVTTDNTSL DDFHVNGGEL ILIHQNPGEF |
| 181 | CVL |

In certain embodiments, the co-stimulatory ligand is CD70. In certain embodiments, the co-stimulatory ligand is human CD70. In certain embodiments, the CD70 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_001243 (SEQ ID NO: 19) or a fragment thereof, and/or may optionally comprise up to one or up to two or up to three conservative amino acid substitutions. SEQ ID NO: 19 is provided below.

| [SEQ ID NO: 19] | |
|---|---|
| 1 | MPEEGSGCSV RRRPYGCVLR AALVPLVAGL VICLVVCIQR FAQAQQQLPL ESLGWDVAEL |
| 61 | QLNHTGPQQD PRLYWQGGPA LGRSFLHGPE LDKGQLRIHR DGIYMVHIQV TLAICSSTTA |
| 121 | SRHHPTTLAV GICSPASRSI SLLRLSFHQG CTIASQRLTP LARGDTLCTN LTGTLLPSRN |
| 181 | TDETFFGVQW VRP |

In certain embodiments, the co-stimulatory ligand is CD86. In certain embodiments, the co-stimulatory ligand is human CD86. In certain embodiments, the CD86 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_787058.5 (SEQ ID NO: 20) or a fragment thereof, and/or may optionally comprise up to one or up to two or up to three conservative amino acid substitutions. SEQ ID NO: 20 is provided below.

[SEQ ID NO: 20]
MDPQCTMGLSNILFVMAFLLSGAAPLKIQAYFNETADLPCQFANSQNQSL
SELVVFWQDQENLVLNEVYLGKEKFDSVHSKYMGRTSFDSDSWILRLHNL
QIKDKGLYQCIIHHKKPIGMIRIHQMNSELSVLANFSQPEIVPISNITEN
VYINLICSSIHGYPEPKKMSVLLRIKNSTIEYDGVMQKSQDNVIELYDVS
ISLSVSFPDVISNMTIFCILETDKIRLLSSPFSIELEDPQPPPDHIPWIT
AVLPTVIICVMVFCLILWKWKKKKRPRNSYKCGINTMEREESEQTKKREK
IHIPERSDEAQRVFKSSKTSSCDKSDTCF

In certain embodiments, the co-stimulatory ligand is GITRL. In certain embodiments, the co-stimulatory ligand is human GITRL. In certain embodiments, the GITRL comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_005083.2 (SEQ ID NO: 21) or a fragment thereof, and/or may optionally comprise up to one or up to two or up to three conservative amino acid substitutions. SEQ ID NO: 21 is provided below.

| [SEQ ID NO: 21] | |
|---|---|
| 1 | MTLHPSPITC EFLFSTALIS PKMCLSHLEN MPLSHSRTQG AQRSSWKLWL FCSIVMLLFL |
| 61 | CSFSWLIFIF LQLETAKEPC MAKFGPLPSK WQMASSEPPC VNKVSDWKLE ILQNGLYLIY |
| 121 | GQVAPNANYN DVAPFEVRLY KNKDMIQTLT NKSKIQNVGG TYELHVGDTI DLIFNSEHQV |
| 181 | LKNNTYWGII LLANPQFIS |

In certain embodiments, the co-stimulatory ligand is ICOS ligand (ICOSLG). In certain embodiments, the co-stimulatory ligand is human ICOSLG. In certain embodiments, the ICOSLG comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_056074.1 (SEQ ID NO: 22) or a fragment thereof, and/or may optionally comprise up to one or up to two or up to three conservative amino acid substitutions. SEQ ID NO:

22 is provided below.

| [SEQ ID NO: 22] | |
|---|---|
| 1 | mrlgspgllf llfsslradt qekevramvg sdvelscacp egsrfdlndv yvywqtsesk |
| 61 | tvvtyhipqn sslenvdsry rnralmspag mlrgdfslrl fnvtpqdeqk fhclvlsgsl |
| 121 | gfqevlsvev tlhvaanfsv pvvsaphsps qdeltftcts ingyprpnvy winktdnsll |
| 181 | dqalqndtvf lnmrglydvv svlriartps vnigccienv llqqnitvgs qtgndigerd |
| 241 | kitenpvstg eknaatwsil avlcllvvva vaigwvcrdr clqhsyagaw ayspeteltg |
| 301 | hv |

In certain embodiments, the co-stimulatory ligand is CD40L (also known as “CD154”). In certain embodiments, the co-stimulatory ligand is human CD40L. In certain embodiments, the CD40L comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_000065.1 (SEQ ID NO: 23) or a fragment thereof, and/or may optionally comprise up to one or up to two or up to three conservative amino acid substitutions. SEQ ID NO: 23 is provided below.

| [SEQ ID NO: 23] | |
|---|---|
| 1 | mietynqtsp rsaatglpis mkifmylltv flitqmigsa lfavylhrrl dkiedernlh |
| 61 | edfvfmktiq rcntgersls llnceeiksq fegfvkdiml nkeetkkens femqkgdqnp |
| 121 | qiaahvisea sskttsvlqw aekgyytmsn nlvtlengkq ltvkrqglyy iyaqvtfcsn |
| 181 | reassqapfi aslclkspgr ferillraan thssakpcgq qsihlggvfe lqpgasvfvn |
| 241 | vtdpsqvshg tgftsfgllk l |

In certain embodiments, the fusion polypeptide comprises an extracellular domain and a transmembrane domain of a second co-stimulatory ligand. In certain embodiments, the fusion polypeptide comprises an extracellular domain and a transmembrane domain of a third co-stimulatory ligand. In certain embodiments, the fusion polypeptide comprises an extracellular domain and a transmembrane domain of a fourth co-stimulatory ligand. In certain embodiments, the fusion polypeptide comprises an extracellular domain and a transmembrane domain of a fifth co-stimulatory ligand. In certain embodiments, the first, second, third, fourth, and fifth co-stimulatory ligands can be the same or different among each other.

In certain embodiments, the fusion polypeptide comprises an extracellular domain and a transmembrane domain of CD80.

5.2.2. Co-Stimulatory Molecules

Non-limiting examples of co-stimulatory molecules include CD28, 4-1BB, OX40, ICOS, DAP-10, CD27, CD40, NKG2D, CD2, and combinations thereof.

In certain embodiments, the co-stimulatory molecule is 4-1BB. 4-1BB can act as a tumor necrosis factor (TNF) ligand and have stimulatory activity. In certain embodiments, the co-stimulatory molecule is human 4-1BB. In certain embodiments, the 4-1BB comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_001552 (SEQ ID NO: 24) or a fragment thereof, and/or may optionally comprise up to one or up to two or up to three conservative amino acid substitutions. SEQ ID NO: 24 is provided below.

| [SEQ ID NO: 24] | |
|---|---|
| 1 | MGNSCYNIVA TLLLVLNFER TRSLQDPCSN CPAGTFCDNN RNQICSPCPP NSFSSAGGQR |
| 61 | TCDICRQCKG VFRTRKECSS TSNAECDCTP GFHCLGAGCS MCEQDCKQGQ ELTKKGCKDC |
| 121 | CFGTFNDQKR GICRPWTNCS LDGKSVLVNG TKERDVVCGP SPADLSPGAS SVTPPAPARE |
| 181 | PGHSPQIISF FLALTSTALL FLLFFLTLRF SVVKRGRKKL LYIFKQPFMR PVQTTQEEDG |
| 241 | CSCRFPEEEE GGCEL |

In certain embodiments, the intracellular domain of 4-1BB comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 3 (or amino acids 214-255 of SEQ ID NO: 24) or a fragment thereof, and/or may optionally comprise up to one or up to two or up to three conservative amino acid substitutions. In certain embodiments, an intracellular domain of 4-1BB comprises or consists of the amino acid sequence set forth in SEQ ID NO: 3 or a functional fragment thereof. A functional fragment of SEQ ID NO: 3 can be a consecutive portion of SEQ ID NO: 3, which is at least about 20, at least about 25, at least about 30, at least about 35, or at least about 40 amino acids in length. In certain embodiments, the functional fragment of SEQ ID NO: 3 retains at least about 80%, at least about 85%, at least about 90%, at least about 95%, or at least about 100% of the primary functions of the intracellular domain of 4-1BB. Non-limiting examples of the primary functions of the intracellular domain of 4-1BB include providing co-stimulatory signaling for the activation and proliferation of an immunoresponsive cell (e.g., a T cell), and interacting and activating downstream adaptors (e.g., TRAFs). In certain embodiments, the intracellular domain of 4-1BB comprises or consists of the amino acid sequence set forth in SEQ ID NO: 3. SEQ ID NO: 3 is provided below:

[SEQ ID NO: 3]
KRGRKKLLYI FKQPFMRPVQ TTQEEDGCSC RFPEEEEGGC EL.

In certain embodiments, the co-stimulatory molecule is CD28. In certain embodiments, the co-stimulatory molecule is human CD28. In certain embodiments, the CD28 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_006130 (SEQ ID NO: 25) or a fragment thereof, and/or may optionally comprise up to one or up to two or up to three conservative amino acid substitutions. SEQ ID NO: 25 is provided below.

| [SEQ ID NO: 25] | |
|---|---|
| 1 | MLRLLLALNL FPSIQVTGNK ILVKQSPMLV AYDNAVNLSC KYSYNLFSRE FRASLHKGLD |
| 61 | SAVEVCVVYG NYSQQLQVYS KTGFNCDGKL GNESVTFYLQ NLYVNQTDIY FCKIEVMYPP |
| 121 | PYLDNEKSNG TIIHVKGKHL CPSPLFPGPS KPFWVLVVVG GVLACYSLLV TVAFIIFWVR |
| 181 | SKRSRLLHSD YMNMTPRRPG PTRKHYQPYA PPRDFAAYRS |

In certain embodiments, the intracellular domain of CD28 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 5 (or amino acids 180 to 219 of SEQ ID NO: 25) or a fragment thereof, and/or may optionally comprise up to one or up to two or up to three conservative amino acid substitutions. In certain embodiments, the intracellular domain of CD28 comprises or consists of the amino acid sequence set forth in SEQ ID NO: 5 or a functional fragment thereof. A functional fragment of SEQ ID NO: 5 can be a consecutive portion of SEQ ID NO: 5, which is at least about 20, at least about 25, at least about 30, or at least about 35 amino acids in length. In certain embodiments, the functional fragment of SEQ ID NO: 5 retains at least about 80%, at least about 85%, at least about 90%, at least about 95%, or at least about 100% of the primary function of the intracellular domain of CD28. Non-limiting examples of the primary functions of the intracellular domain of CD28 include providing co-stimulatory signaling for the activation and proliferation of an immunoresponsive cell (e.g., a T cell), and interacting with protein adaptors (e.g., PI3K, GRB2, and LCK). In certain embodiments, the intracellular domain of CD28 comprises or consists of the amino acid sequence set forth in SEQ ID NO: 5. SEQ ID NO: 5 is provided below:

[SEQ ID NO: 5]
RSERSRLLHSDYMNMTPRRPGPTREHYQPYAPPRDFAAYR

In certain embodiments, the co-stimulatory molecule is OX40. In certain embodiments, the co-stimulatory molecule is human OX40. In certain embodiments, the OX40 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_003318.1 (SEQ ID NO: 26) or a fragment thereof, and/or may optionally comprise up to one or up to two or up to three conservative amino acid substitutions. In certain embodiments, the intracellular domain of OX40 comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 236 to 277 of SEQ ID NO: 26 or a fragment thereof, and/or may optionally comprise up to one or up to two or up to three conservative amino acid substitutions. In certain embodiments, the intracellular domain of OX40 comprises or consists of amino acids 236 to 277 of SEQ ID NO: 26 or a fragment thereof. In certain embodiments, the intracellular domain of OX40 comprises or consists of the amino acids 236 to 277 of SEQ ID NO: 26. SEQ ID NO: 26 is provided below.

| [SEQ ID NO: 26] | |
|---|---|
| 1 | MCVGARRLGR GPCAALLLLG LGLSTVTGLH CVGDTYPSND RCCHECRPGN GMVSRCSRSQ |
| 61 | NTVCRPCGPG FYNDVVSSKP CKPCTWCNLR SGSERKQLCT ATQDTVCRCR AGTQPLDSYK |
| 121 | PGVDCAPCPP GHFSPGDNQA CKPWTNCTLA GKHTLQPASN SSDAICEDRD PPATQPQETQ |
| 181 | GPPARPITVQ PTEAWPRTSQ GPSTRPVEVP GGRAVAAILG LGLVLGLLGP LAILLALYLL |
| 241 | RRDQRLPPDA HKPPGGGSFR TPIQEEQADA HSTLAKI |

In certain embodiments, the co-stimulatory molecule is ICOS. In certain embodiments, the co-stimulatory molecule is human ICOS. In certain embodiments, the ICOS comprises or consists of an amino acid sequence that is at least about 80%, at least about 85%, at least about 90%, at least about 95%, at least about 96%, at least about 97%, at least about 98%, or at least about 99%, at least about 100% homologous or identical homologous to the amino acid sequence having a NCBI Reference No: NP_036224.1 (SEQ ID NO: 27) or a fragment thereof, and/or may optionally comprise up to one or up to two or up to three conservative amino acid substitutions. In certain embodiments, the intracellular domain of ICOS comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 162 to 199 of SEQ ID NO: 27 or a fragment thereof, and/or may optionally comprise up to one or up to two or up to three conservative amino acid substitutions. In certain embodiments, the intracellular domain of ICOS comprises or consists of amino acids 162 to 199 of SEQ ID NO: 27 or a fragment thereof. In certain embodiments, the intracellular domain of ICOS comprises or consists of the amino acids 162 to 199 of SEQ ID NO: 27. SEQ ID NO: 27 is provided below.

| [SEQ ID NO: 27] | |
|---|---|
| 1 | MKSGLWYFFL FCLRIKVLTG EINGSANYEM FIFHNGGVQI LCKYPDIVQQ FKMQLLKGGQ |
| 61 | ILCDLIKTKG SGNTVSIKSL KFCHSQLSNN SVSFFLYNLD HSHANYYFCN LSIFDPPPFK |
| 121 | VTLIGGYLHI YESQLCCQLK FWLPIGCAAF VVVCILGCIL ICWLTKKKYS SSVHDPNGEY |
| 181 | MFMRAVNTAK KSRLTDVTL |

In certain embodiments, the co-stimulatory molecule is DAP-10. In certain embodiments, the co-stimulatory molecule is human DAP-10. In certain embodiments, the DAP-10 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_001007470.1 (SEQ ID NO: 28) or a fragment thereof, and/or may optionally comprise up to one or up to two or up to three conservative amino acid substitutions. In certain embodiments, the intracellular domain of DAP10 comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 70 to 92 of SEQ ID NO: 28 or a fragment thereof, and/or may optionally comprise up to one or up to two or up to three conservative amino acid substitutions. In certain embodiments, the intracellular domain of DAP10 comprises or consists of amino acids 70 to 92 of SEQ ID NO: 28 or a fragment thereof. In certain embodiments, the intracellular domain of DAP10 comprises or consists of the amino acids 70 to 92 of SEQ ID NO: 28. SEQ ID NO: 28 is provided below.

| [SEQ ID NO: 28] | |
|---|---|
| 1 | MIHLGHILFLLLLPVAAAQTTPGERSSLPAFYPGTSGSCS GCGSLSLPLL AGLVAADAVA |
| 61 | SLLIVGAVFL CARPRRSPAQ DGKVYINMPG RG |

In certain embodiments, the co-stimulatory molecule is CD27. In certain embodiments, the co-stimulatory molecule is human CD27. In certain embodiments, the CD27 comprises or consists of an amino acid sequence that is at least about 80%, at least about 85%, at least about 90%, at least about 95%, at least about 96%, at least about 97%, at least about 98%, or at least about 99%, at least about 100% homologous or identical homologous to the amino acid sequence having a NCBI Reference No: NP_001233.1 (SEQ ID NO: 29) or a fragment thereof, and/or may optionally comprise up to one or up to two or up to three conservative amino acid substitutions. In certain embodiments, the intracellular domain of CD27 comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 213 to 260 of SEQ ID NO: 29 or a fragment thereof, and/or may optionally comprise up to one or up to two or up to three conservative amino acid substitutions. In certain embodiments, the intracellular domain of CD27 comprises or consists of amino acids 213 to 260 of SEQ ID NO: 29 or a fragment thereof. In certain embodiments, the intracellular domain of CD27 comprises or consists of the amino acids 213 to 260 of SEQ ID NO: 29. SEQ ID NO: 29 is provided below.

| [SEQ ID NO: 29] | |
|---|---|
| 1 | MARPHPWWLC VLGTLVGLSA TPAPKSCPER HYWAQGKLCC QMCEPGTFLV KDCDQHRKAA |
| 61 | QCDPCIPGVS FSPDHHTRPH CESCRHCNSG LLVRNCTITA NAECACRNGW QCRDKECTEC |
| 121 | DPLPNPSLTA RSSQALSPHP QPTHLPYVSE MLEARTAGHM QTLADFRQLP ARTLSTHWPP |
| 181 | QRSLCSSDFI RILVIFSGMF LVFTLAGALF LHQRRKYRSN KGESPVEPAE PCRYSCPREE |
| 241 | EGSTIPIQED YRKPEPACSP |

In certain embodiments, the co-stimulatory molecule is CD40. In certain embodiments, the co-stimulatory molecule is human CD40. In certain embodiments, the CD40 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_001241.1 (SEQ ID NO: 30) or a fragment thereof, and/or may optionally comprise up to one or up to two or up to three conservative amino acid substitutions. In certain embodiments, the intracellular domain of CD40 comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 216 to 277 of SEQ ID NO: 30 or a fragment thereof, and/or may optionally comprise up to one or up to two or up to three conservative amino acid substitutions. In certain embodiments, the intracellular domain of CD40 comprises or consists of amino acids 216 to 277 of SEQ ID NO: 30 or a fragment thereof. In certain embodiments, the intracellular domain of CD40 comprises or consists of the amino acids 216 to 277 of SEQ ID NO: 30. SEQ ID NO: 30 is provided below.

| [SEQ ID NO: 30] | |
|---|---|
| 1 | MVRLPLQCVL WGCLLTAVHP EPPTACREKQ YLINSQCCSL CQPGQKLVSD CTEFTETECL |
| 61 | PCGESEFLDT WNRETHCHQH KYCDPNLGLR VQQKGTSETD TICTCEEGWH CTSEACESCV |
| 121 | LHRSCSPGFG VKQIATGVSD TICEPCPVGF FSNVSSAFEK CHPWTSCETK DLVVQQAGTN |
| 181 | KTDVVCGPQD RLRALVVIPI IFGILFAILL VLVFIKKVAK KPTNKAPHPK QEPQEINFPD |
| 241 | DLPGSNTAAP VQETLHGCQP VTQEDGKESR ISVQERQ |

In certain embodiments, the co-stimulatory molecule is NKG2D. In certain embodiments, the co-stimulatory molecule is human NKG2D. In certain embodiments, the NKG2D comprises or consists of an amino acid sequence that is at least about 80%, at least about 85%, at least about 90%, at least about 95%, at least about 96%, at least about 97%, at least about 98%, or at least about 99%, at least about 100% homologous or identical homologous to the amino acid sequence having a NCBI Reference No: NP_031386.2 (SEQ ID NO: 31) or a fragment thereof, and/or may optionally comprise up to one or up to two or up to three conservative amino acid substitutions. In certain embodiments, the intracellular domain of NKG2D comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 73 to 216 of SEQ ID NO: 31 or a fragment thereof, and/or may optionally comprise up to one or up to two or up to three conservative amino acid substitutions. In certain embodiments, the intracellular domain of NKG2D comprises or consists of amino acids 73 to 216 of SEQ ID NO: 31 or a fragment thereof. In certain embodiments, the intracellular domain of NKG2D comprises or consists of the amino acids 73 to 216 of SEQ ID NO: 31. SEQ ID NO: 31 is provided below.

| [SEQ ID NO: 31] | |
|---|---|
| 1 | MGWIRGRRSR HSWEMSEFHN YNLDLKKSDF STRWQKQRCP VVKSKCRENA SPFFFCCFIA |
| 61 | VAMGIRFIIM VTIWSAVFLN SLFNQEVQIP LTESYCGPCP KNWICYKNNC YQFFDESKNW |
| 121 | YESQASCMSQ NASLLKVYSK EDQDLLKLVK SYHWMGLVHI PTNGSWQWED GSILSPNLLT |
| 181 | IIEMQKGDCA LYASSFKGYI ENCSTPNTYI CMQRTV |

In certain embodiments, the co-stimulatory molecule is CD2. In certain embodiments, the co-stimulatory molecule is human CD2. In certain embodiments, CD2 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_001315538 (SEQ ID NO: 32) or a fragment thereof, and/or may optionally comprise up to one or up to two or up to three conservative amino acid substitutions. In certain embodiments, the intracellular domain of CD2 comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 236 to 351 of SEQ ID NO: 32 or a fragment thereof, and/or may optionally comprise up to one or up to two or up to three conservative amino acid substitutions. In certain embodiments, the intracellular domain of CD2 comprises or consists of amino acids amino acids 236 to 351 of SEQ ID NO: 32 or a fragment thereof. In certain embodiments, the intracellular domain of CD2 comprises or consists of the amino acids 236 to 351 of SEQ ID NO: 32. SEQ ID NO: 32 is provided below.

| [SEQ ID NO: 32] | |
|---|---|
| 1 | msfpckfvas fllifnvssk gayskeitna letwgalgqd inldipsfqm sddiddikwe |
| 61 | ktsdkkkiaq frkeketfke kdtyklfkng tlkikhlktd dqdiykvsiy dtkgknvlek |
| 121 | ifdlkiqery skpkiswtci nttltcevmn gtdpelnlyq dgkhlklsqr vithkwttsl |
| 181 | sakfkctagn kvskessvep vscpggsilg qsnglsawtp pshptslpfa ekgldiylii |
| 241 | gicgggsllm vfvallvfyi tkrkkqrsrr ndeeletrah rvateergrk phqipastpq |
| 301 | npatsqhppp ppghrsqaps hrppppghry qhqpqkrppa psgtqvhqqk gpplprprvq |
| 361 | pkpphgaaen slspssn |

In certain embodiments, the fusion polypeptide comprises an intracellular domain of a second co-stimulatory molecule. In certain embodiments, the fusion polypeptide comprises an intracellular domain of a third co-stimulatory molecule. In certain embodiments, the fusion polypeptide comprises an intracellular domain of a fourth co-stimulatory molecule. In certain embodiments, the fusion polypeptide comprises an intracellular domain of a fifth co-stimulatory molecule. In certain embodiments, the first, second, third, fourth, and fifth co-stimulatory molecule can be the same or different among each other.

In certain embodiments, the fusion polypeptide comprises an intracellular domain of a first co-stimulatory molecule that is 4-1BB, and an intracellular domain of a second co-stimulatory molecule that is CD28.

5.2.3. Cytokine Receptors

In certain embodiments, the fusion polypeptide further comprises a cytokine receptor. In certain embodiments, the fusion polypeptide further comprises a signaling domain of a cytokine receptor. The cytokine receptor or the signaling domain of the cytokine receptor can be positioned at the N-terminus or C-terminus of the fusion polypeptide. In certain embodiments, the cytokine receptor or the signaling domain of the cytokine receptor is positioned at the C-terminus of the fusion polypeptide. In certain embodiments, the cytokine receptor or the signaling domain of the cytokine receptor is positioned at the C-terminus of the co-stimulatory ligand (e.g., CD80, 4-1BBL, OX40L, CD70, GITRL, CD40L, CD86, or ICOSLG). Non-limiting examples of cytokine receptors include CD121a, CDw121b, IL-18Ra, IL18Rb, CD122, CD25, CD132, CD124, CD213a13, CD127, IL-9R, IL15Ra, CDw125, CDw131, CD126, CD130, IL11Ra, Cd114, CD212, CD4, CDw217, CD118, and CDw119.

5.2.4. Exemplary Fusion Polypeptides

In certain embodiments, the fusion polypeptide comprises: a) an extracellular domain and a transmembrane domain of a co-stimulatory ligand, wherein the co-stimulatory ligand is CD80; and b) an intracellular domain of a first co-stimulatory molecule, wherein the first co-stimulatory molecule is selected from the group consisting of 4-1BB, CD28, OX40, ICOS, DAP-10, CD27, CD40, NKG2D, CD2, and combinations thereof.

5.2.4.1. CD80/4-1BB Fusion Polypeptide

In certain embodiments, the fusion polypeptide comprises: a) an extracellular domain and a transmembrane domain of a co-stimulatory ligand, wherein the co-stimulatory ligand is CD80; and b) an intracellular domain of a first co-stimulatory molecule, wherein the first co-stimulatory molecule is 4-1BB.

In certain embodiment, the CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 16. In certain embodiments, the CD80 comprises or consists of the amino acid sequence set forth in SEQ ID NO: 16.

In certain embodiments, the extracellular domain of CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 1 (or amino acids 1-242 of SEQ ID NO: 16). In certain embodiments, the extracellular domain of CD80 comprises or consists of the amino acid sequence set forth in SEQ ID NO: 1 or a functional fragment thereof. In certain embodiments, the extracellular domain of CD80 comprises or consists of the amino acid sequence set forth in SEQ ID NO: 1 (or amino acids 1-242 of SEQ ID NO: 16).

In certain embodiments, the transmembrane domain of CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 2 (or amino acids 243-263 of SEQ ID NO: 16). In certain embodiments, the transmembrane domain of CD80 comprises or consists of the amino acid sequence set forth in SEQ ID NO: 2 or a fragment thereof. In certain embodiments, the transmembrane domain of CD80 comprises or consists of the amino acid sequence set forth in SEQ ID NO: 2 (or amino acids 243-263 of SEQ ID NO: 16).

In certain embodiment, the 4-1BB comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 24. In certain embodiments, the 4-1BB comprises or consists of the amino acid sequence set forth in SEQ ID NO: 24.

In certain embodiments, the intracellular domain of 4-1BB comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 3 (or amino acids 214-255 of SEQ ID NO: 24). In certain embodiments, the intracellular domain of 4-1BB comprises or consists of the amino acid sequence set forth in SEQ ID NO: 3 or a functional fragment thereof. In certain embodiments, the intracellular domain of 4-1BB comprises or consists of the amino acid sequence set forth in SEQ ID NO: 3 (or amino acids 214-255 of SEQ ID NO: 24).

In certain embodiments, the fusion polypeptide comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 4. In certain embodiments, the fusion polypeptide comprises or consists of the amino acid sequence set forth in SEQ ID NO: 4. SEQ ID NO: 4 is provided below.

[SEQ ID NO: 4]
MGHTRRQGTSPSKCPYLNEFQLLVLAGLSHFCSGVIHVTKEVKEVATLS
CGHNVSVEELAQTRIYWQKEKKMVLTMMSGDMNIWPEYKNRTIFDITNN
LSIVILALRPSDEGTYECVVLKYEKDAFKREHLAEVTLSVKADEPTPSI
SDFEIPTSNIRRIICSTSGGFPEPHLSWLENGEELNAINTTVSQDPETE
LYAVSSKLDFNMTTNHSFMCLIKYGHLRVNQTENWNTTKQEHFPDNLLP
SWAITLISVNGIEVICCLTYCFKRGRKKLLYIFKQPFMRPVQTTQEEDG
CSCREPEEEEGGCEL

5.2.4.2. CD80/4-1BB-CD28 Fusion Polypeptide

In certain embodiments, the fusion polypeptide comprises a) an extracellular domain and a transmembrane domain of a co-stimulatory ligand, wherein the co-stimulatory ligand is CD80; b) an intracellular domain of a first co-stimulatory molecule, wherein the first co-stimulatory molecule is 4-1BB; and c) an intracellular domain of a second co-stimulatory molecule, wherein the second co-stimulatory molecule is CD28.

In certain embodiment, the CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 16. In certain embodiments, the CD80 comprises or consists of the amino acid sequence set forth in SEQ ID NO: 16.

In certain embodiments, the extracellular domain of CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 1 (or amino acids 1-242 of SEQ ID NO: 16). In certain embodiments, the extracellular domain of CD80 comprises or consists of the amino acid sequence set forth in SEQ ID NO: 1 or a functional fragment thereof. In certain embodiments, the extracellular domain of CD80 comprises or consists of the amino acid sequence set forth in SEQ ID NO: 1 (or amino acids 1-242 of SEQ ID NO: 16).

In certain embodiments, the transmembrane domain of CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 2 (or amino acids 243-263 of SEQ ID NO: 16). In certain embodiments, the transmembrane domain of CD80 comprises or consists of the amino acid sequence set forth in SEQ ID NO: 2 or a fragment thereof. In certain embodiments, the transmembrane domain of CD80 comprises or consists of the amino acid sequence set forth in SEQ ID NO: 2 (or amino acids 243-263 of SEQ ID NO: 16).

In certain embodiment, the 4-1BB comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 24. In certain embodiments, the 4-1BB comprises or consists of the amino acid sequence set forth in SEQ ID NO: 24.

In certain embodiments, the intracellular domain of 4-1BB comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 3 (or amino acids 214-255 of SEQ ID NO: 24). In certain embodiments, the intracellular domain of 4-1BB comprises or consists of the amino acid sequence set forth in SEQ ID NO: 3 or a functional fragment thereof. In certain embodiments, the intracellular domain of 4-1BB comprises or consists of the amino acid sequence set forth in SEQ ID NO: 3 (or amino acids 214-255 of SEQ ID NO: 24).

In certain embodiment, the CD28 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 25. In certain embodiments, the CD28 comprises or consists of the amino acid sequence set forth in SEQ ID NO: 25.

In certain embodiments, the intracellular domain of CD28 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 5 (or amino acids 180 to 219 of SEQ ID NO: 25). In certain embodiments, the intracellular domain of CD28 comprises or consists of the amino acid sequence set forth in SEQ ID NO: 5 or a functional fragment thereof. In certain embodiments, the intracellular domain of CD28 comprises or consists of the amino acid sequence set forth in SEQ ID NO: 5 (or amino acids 180 to 219 of SEQ ID NO: 25).

In certain embodiments, the fusion polypeptide comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 6. In certain embodiments, the fusion polypeptide comprises or consists of the amino acid sequence set forth in SEQ ID NO: 6. SEQ ID NO: 6 is provided below.

[SEQ ID NO: 6]
MGHTRRQGTSPSKCPYLNFFQLLVLAGLSHFCSGVIHVTKEVKEVATLS
CGHNVSVEELAQTRIYWQKEKKMVLTMMSGDMNIWPEYKNRTIFDITNN
LSIVILALRPSDEGTYECVVLKYEKDAFKREHLAEVTLSVKADEPTPSI
SDFEIPTSNIRRIICSTSGGFPEPHLSWLENGEELNAINTTVSQDPETE
LYAVSSKLDFNMTTNHSFMCLIKYGHLRVNQTFNWNTTKQEHFPDNLLP
SWAITLISVNGIFVICCLTYCFRSKRSRLLHSDYMNMTPRRPGPTRKHY
QPYAPPRDFAAYRKRGRKKLLYIFKQPFMRPVQTTQEEDGCSCRFPEEE
EGGCEL

5.2.4.3. CD80/CD28 Fusion Polypeptide

In certain embodiments, the fusion polypeptide comprises: a) an extracellular domain and a transmembrane domain of a co-stimulatory ligand, wherein the co-stimulatory ligand is CD80; and b) an intracellular domain of a first co-stimulatory molecule, wherein the first co-stimulatory molecule is CD28.

In certain embodiment, the CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 16. In certain embodiments, the CD80 comprises or consists of the amino acid sequence set forth in SEQ ID NO: 16.

In certain embodiments, the extracellular domain of CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 1 (or amino acids 1-242 of SEQ ID NO: 16). In certain embodiments, the extracellular domain of CD80 comprises or consists of the amino acid sequence set forth in SEQ ID NO: 1 or a functional fragment thereof. In certain embodiments, the extracellular domain of CD80 comprises or consists of the amino acid sequence set forth in SEQ ID NO: 1 (or amino acids 1-242 of SEQ ID NO: 16).

In certain embodiments, the transmembrane domain of CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 2 (or amino acids 243-263 of SEQ ID NO: 16). In certain embodiments, the transmembrane domain of CD80 comprises or consists of the amino acid sequence set forth in SEQ ID NO: 2 or a fragment thereof. In certain embodiments, the transmembrane domain of CD80 comprises or consists of the amino acid sequence set forth in SEQ ID NO: 2 (or amino acids 243-263 of SEQ ID NO: 16).

In certain embodiment, the CD28 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 25. In certain embodiments, the CD28 comprises or consists of the amino acid sequence set forth in SEQ ID NO: 25.

In certain embodiments, the intracellular domain of CD28 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 5 (or amino acids 180 to 219 of SEQ ID NO: 25). In certain embodiments, the intracellular domain of CD28 comprises or consists of the amino acid sequence set forth in SEQ ID NO: 5 or a functional fragment thereof. In certain embodiments, the intracellular domain of CD28 comprises or consists of the amino acid sequence set forth in SEQ ID NO: 5 (or amino acids 180 to 219 of SEQ ID NO: 25).

5.2.4.4. CD80/OX40 Fusion Polypeptide

In certain embodiments, the fusion polypeptide comprises: a) an extracellular domain and a transmembrane domain of a co-stimulatory ligand, wherein the co-stimulatory ligand is CD80; and b) an intracellular domain of a first co-stimulatory molecule, wherein the first co-stimulatory molecule is OX40.

In certain embodiment, the CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 16. In certain embodiments, the CD80 comprises or consists of the amino acid sequence set forth in SEQ ID NO: 16.

In certain embodiments, the extracellular domain of CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 1 (or amino acids 1-242 of SEQ ID NO: 16). In certain embodiments, the extracellular domain of CD80 comprises or consists of the amino acid sequence set forth in SEQ ID NO: 1 or a functional fragment thereof. In certain embodiments, the extracellular domain of CD80 comprises or consists of the amino acid sequence set forth in SEQ ID NO: 1 (or amino acids 1-242 of SEQ ID NO: 16).

In certain embodiments, the transmembrane domain of CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 2 (or amino acids 243-263 of SEQ ID NO: 16). In certain embodiments, the transmembrane domain of CD80 comprises or consists of the amino acid sequence set forth in SEQ ID NO: 2 or a fragment thereof. In certain embodiments, the transmembrane domain of CD80 comprises or consists of the amino acid sequence set forth in SEQ ID NO: 2 (or amino acids 243-263 of SEQ ID NO: 16).

In certain embodiments, the OX40 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 26 or a fragment thereof, and/or may optionally comprise up to one or up to two or up to three conservative amino acid substitutions. In certain embodiments, the intracellular domain of OX40 comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 236 to 277 of SEQ ID NO: 26 or a fragment thereof, and/or may optionally comprise up to one or up to two or up to three conservative amino acid substitutions. In certain embodiments, the intracellular domain of OX40 comprises or consists of amino acids 236 to 277 of SEQ ID NO: 26 or a fragment thereof. In certain embodiments, the intracellular domain of OX40 comprises or consists of amino acids 236 to 277 of SEQ ID NO: 26.

5.2.4.5. CD80/ICOS Fusion Polypeptide

In certain embodiments, the fusion polypeptide comprises: a) an extracellular domain and a transmembrane domain of a co-stimulatory ligand, wherein the co-stimulatory ligand is CD80; and b) an intracellular domain of a first co-stimulatory molecule, wherein the first co-stimulatory molecule is ICOS.

In certain embodiment, the CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 16. In certain embodiments, the CD80 comprises or consists of the amino acid sequence set forth in SEQ ID NO: 16.

In certain embodiments, the extracellular domain of CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 1 (or amino acids 1-242 of SEQ ID NO: 16). In certain embodiments, the extracellular domain of CD80 comprises or consists of the amino acid sequence set forth in SEQ ID NO: 1 or a functional fragment thereof. In certain embodiments, the extracellular domain of CD80 comprises or consists of the amino acid sequence set forth in SEQ ID NO: 1 (or amino acids 1-242 of SEQ ID NO: 16).

In certain embodiments, the transmembrane domain of CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 2 (or amino acids 243-263 of SEQ ID NO: 16). In certain embodiments, the transmembrane domain of CD80 comprises or consists of the amino acid sequence set forth in SEQ ID NO: 2 or a fragment thereof. In certain embodiments, the transmembrane domain of CD80 comprises or consists of the amino acid sequence set forth in SEQ ID NO: 2 (or amino acids 243-263 of SEQ ID NO: 16).

In certain embodiments, the ICOS comprises or consists of an amino acid sequence that is at least about 80%, at least about 85%, at least about 90%, at least about 95%, at least about 96%, at least about 97%, at least about 98%, or at least about 99%, at least about 100% homologous or identical homologous to the amino acid sequence set forth in SEQ ID NO: 27 or a fragment thereof, and/or may optionally comprise up to one or up to two or up to three conservative amino acid substitutions. In certain embodiments, the intracellular domain of ICOS comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 162 to 199 of SEQ ID NO: 27 or a fragment thereof, and/or may optionally comprise up to one or up to two or up to three conservative amino acid substitutions. In certain embodiments, the intracellular domain of ICOS comprises or consists of amino acids 162 to 199 of SEQ ID NO: 27 or a fragment thereof. In certain embodiments, the intracellular domain of ICOS comprises or consists of amino acids 162 to 199 of SEQ ID NO: 27.

5.2.4.6. CD80/DAP-10 Fusion Polypeptide

In certain embodiments, the fusion polypeptide comprises: a) an extracellular domain and a transmembrane domain of a co-stimulatory ligand, wherein the co-stimulatory ligand is CD80; and b) an intracellular domain of a first co-stimulatory molecule, wherein the first co-stimulatory molecule is DAP-10.

In certain embodiment, the CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 16. In certain embodiments, the CD80 comprises or consists of the amino acid sequence set forth in SEQ ID NO: 16.

In certain embodiments, the extracellular domain of CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 1 (or amino acids 1-242 of SEQ ID NO: 16). In certain embodiments, the extracellular domain of CD80 comprises or consists of the amino acid sequence set forth in SEQ ID NO: 1 or a functional fragment thereof. In certain embodiments, the extracellular domain of CD80 comprises or consists of the amino acid sequence set forth in SEQ ID NO: 1 (or amino acids 1-242 of SEQ ID NO: 16).

In certain embodiments, the transmembrane domain of CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 2 (or amino acids 243-263 of SEQ ID NO: 16). In certain embodiments, the transmembrane domain of CD80 comprises or consists of the amino acid sequence set forth in SEQ ID NO: 2 or a fragment thereof. In certain embodiments, the transmembrane domain of CD80 comprises or consists of the amino acid sequence set forth in SEQ ID NO: 2 (or amino acids 243-263 of SEQ ID NO: 16).

In certain embodiments, the DAP-10 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 28 or a fragment thereof, and/or may optionally comprise up to one or up to two or up to three conservative amino acid substitutions. In certain embodiments, the intracellular domain of DAP10 comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 70 to 92 of SEQ ID NO: 28 or a fragment thereof, and/or may optionally comprise up to one or up to two or up to three conservative amino acid substitutions. In certain embodiments, the intracellular domain of DAP10 comprises or consists of amino acids 70 to 92 of SEQ ID NO: 28 or a fragment thereof. In certain embodiments, the intracellular domain of DAP10 comprises or consists of amino acids 70 to 92 of SEQ ID NO: 28.

5.2.4.7. CD80/CD27 Fusion Polypeptide

In certain embodiments, the fusion polypeptide comprises: a) an extracellular domain and a transmembrane domain of a co-stimulatory ligand, wherein the co-stimulatory ligand is CD80; and b) an intracellular domain of a first co-stimulatory molecule, wherein the first co-stimulatory molecule is CD27.

In certain embodiment, the CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 16. In certain embodiments, the CD80 comprises or consists of the amino acid sequence set forth in SEQ ID NO: 16.

In certain embodiments, the extracellular domain of CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 1 (or amino acids 1-242 of SEQ ID NO: 16). In certain embodiments, the extracellular domain of CD80 comprises or consists of the amino acid sequence set forth in SEQ ID NO: 1 or a functional fragment thereof. In certain embodiments, the extracellular domain of CD80 comprises or consists of the amino acid sequence set forth in SEQ ID NO: 1 (or amino acids 1-242 of SEQ ID NO: 16).

In certain embodiments, the transmembrane domain of CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 2 (or amino acids 243-263 of SEQ ID NO: 16). In certain embodiments, the transmembrane domain of CD80 comprises or consists of the amino acid sequence set forth in SEQ ID NO: 2 or a fragment thereof. In certain embodiments, the transmembrane domain of CD80 comprises or consists of the amino acid sequence set forth in SEQ ID NO: 2 (or amino acids 243-263 of SEQ ID NO: 16).

In certain embodiments, the CD27 comprises or consists of an amino acid sequence that is at least about 80%, at least about 85%, at least about 90%, at least about 95%, at least about 96%, at least about 97%, at least about 98%, or at least about 99%, at least about 100% homologous or identical homologous to the amino acid sequence set forth in SEQ ID NO: 29 or a fragment thereof, and/or may optionally comprise up to one or up to two or up to three conservative amino acid substitutions. In certain embodiments, the intracellular domain of CD27 comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 213 to 260 of SEQ ID NO: 29 or a fragment thereof, and/or may optionally comprise up to one or up to two or up to three conservative amino acid substitutions. In certain embodiments, the intracellular domain of CD27 comprises or consists of amino acids 213 to 260 of SEQ ID NO: 29 or a fragment thereof. In certain embodiments, the intracellular domain of CD27 comprises or consists of amino acids 213 to 260 of SEQ ID NO: 29.

5.2.4.8. CD80/CD40 Fusion Polypeptide

In certain embodiments, the fusion polypeptide comprises: a) an extracellular domain and a transmembrane domain of a co-stimulatory ligand, wherein the co-stimulatory ligand is CD80; and b) an intracellular domain of a first co-stimulatory molecule, wherein the first co-stimulatory molecule is CD40.

In certain embodiment, the CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 16. In certain embodiments, the CD80 comprises or consists of the amino acid sequence set forth in SEQ ID NO: 16.

In certain embodiments, the extracellular domain of CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 1 (or amino acids 1-242 of SEQ ID NO: 16). In certain embodiments, the extracellular domain of CD80 comprises or consists of the amino acid sequence set forth in SEQ ID NO: 1 or a functional fragment thereof. In certain embodiments, the extracellular domain of CD80 comprises or consists of the amino acid sequence set forth in SEQ ID NO: 1 (or amino acids 1-242 of SEQ ID NO: 16).

In certain embodiments, the transmembrane domain of CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 2 (or amino acids 243-263 of SEQ ID NO: 16). In certain embodiments, the transmembrane domain of CD80 comprises or consists of the amino acid sequence set forth in SEQ ID NO: 2 or a fragment thereof. In certain embodiments, the transmembrane domain of CD80 comprises or consists of the amino acid sequence set forth in SEQ ID NO: 2 (or amino acids 243-263 of SEQ ID NO: 16).

In certain embodiments, the CD40 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 30 or a fragment thereof, and/or may optionally comprise up to one or up to two or up to three conservative amino acid substitutions. In certain embodiments, the intracellular domain of CD40 comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 216 to 277 of SEQ ID NO: 30 or a fragment thereof, and/or may optionally comprise up to one or up to two or up to three conservative amino acid substitutions. In certain embodiments, the intracellular domain of CD40 comprises or consists of amino acids 216 to 277 of SEQ ID NO: 30 or a fragment thereof. In certain embodiments, the intracellular domain of CD40 comprises or consists of the amino acids 216 to 277 of SEQ ID NO: 30.

5.2.4.9. CD80/NKG2D Fusion Polypeptide

In certain embodiments, the fusion polypeptide comprises: a) an extracellular domain and a transmembrane domain of a co-stimulatory ligand, wherein the co-stimulatory ligand is CD80; and b) an intracellular domain of a first co-stimulatory molecule, wherein the first co-stimulatory molecule is NKG2D.

In certain embodiment, the CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 16. In certain embodiments, the CD80 comprises or consists of the amino acid sequence set forth in SEQ ID NO: 16.

In certain embodiments, the extracellular domain of CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 1 (or amino acids 1-242 of SEQ ID NO: 16). In certain embodiments, the extracellular domain of CD80 comprises or consists of the amino acid sequence set forth in SEQ ID NO: 1 or a functional fragment thereof. In certain embodiments, the extracellular domain of CD80 comprises or consists of the amino acid sequence set forth in SEQ ID NO: 1 (or amino acids 1-242 of SEQ ID NO: 16).

In certain embodiments, the transmembrane domain of CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 2 (or amino acids 243-263 of SEQ ID NO: 16). In certain embodiments, the transmembrane domain of CD80 comprises or consists of the amino acid sequence set forth in SEQ ID NO: 2 or a fragment thereof. In certain embodiments, the transmembrane domain of CD80 comprises or consists of the amino acid sequence set forth in SEQ ID NO: 2 (or amino acids 243-263 of SEQ ID NO: 16).

In certain embodiments, the NKG2D comprises or consists of an amino acid sequence that is at least about 80%, at least about 85%, at least about 90%, at least about 95%, at least about 96%, at least about 97%, at least about 98%, or at least about 99%, at least about 100% homologous or identical homologous to the amino acid sequence set forth in SEQ ID NO: 31 or a fragment thereof, and/or may optionally comprise up to one or up to two or up to three conservative amino acid substitutions. In certain embodiments, the intracellular domain of NKG2D comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 73 to 216 of SEQ ID NO: 31 or a fragment thereof, and/or may optionally comprise up to one or up to two or up to three conservative amino acid substitutions. In certain embodiments, the intracellular domain of NKG2D comprises or consists of amino acids 73 to 216 of SEQ ID NO: 31 or a fragment thereof. In certain embodiments, the intracellular domain of NKG2D comprises or consists of amino acids 73 to 216 of SEQ ID NO: 31.

5.2.4.9. CD80/CD2 Fusion Polypeptide

In certain embodiments, the fusion polypeptide comprises: a) an extracellular domain and a transmembrane domain of a co-stimulatory ligand, wherein the co-stimulatory ligand is CD80; and b) an intracellular domain of a first co-stimulatory molecule, wherein the first co-stimulatory molecule is CD2.

In certain embodiment, the CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 16. In certain embodiments, the CD80 comprises or consists of the amino acid sequence set forth in SEQ ID NO: 16.

In certain embodiments, the extracellular domain of CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 1 (or amino acids 1-242 of SEQ ID NO: 16). In certain embodiments, the extracellular domain of CD80 comprises or consists of the amino acid sequence set forth in SEQ ID NO: 1 or a functional fragment thereof. In certain embodiments, the extracellular domain of CD80 comprises or consists of the amino acid sequence set forth in SEQ ID NO: 1 (or amino acids 1-242 of SEQ ID NO: 16).

In certain embodiments, the transmembrane domain of CD80 comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 2 (or amino acids 243-263 of SEQ ID NO: 16). In certain embodiments, the transmembrane domain of CD80 comprises or consists of the amino acid sequence set forth in SEQ ID NO: 2 or a fragment thereof. In certain embodiments, the transmembrane domain of CD80 comprises or consists of the amino acid sequence set forth in SEQ ID NO: 2 (or amino acids 243-263 of SEQ ID NO: 16).

In certain embodiments, the CD2 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 32 or a fragment thereof, and/or may optionally comprise up to one or up to two or up to three conservative amino acid substitutions. In certain embodiments, the intracellular domain of CD2 comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 236 to 351 of SEQ ID NO: 32 or a fragment thereof, and/or may optionally comprise up to one or up to two or up to three conservative amino acid substitutions. In certain embodiments, the intracellular domain of CD2 comprises or consists of amino acids 236 to 351 of SEQ ID NO: 32 or a fragment thereof. In certain embodiments, the intracellular domain of CD2 comprises or consists of amino acids 236 to 351 of SEQ ID NO: 32.

5.2.5. Activities of Fusion Polypeptides

The presently disclosed fusion polypeptide is capable of stimulating a cell comprising an antigen-recognizing receptor (e.g., a CAR, a TCR, or a TCR like molecule). In certain embodiments, the fusion polypeptide provides stimulation to T cells in cis and/or in trans.

In certain embodiments, the co-stimulatory ligand comprised in a presently disclosed fusion polypeptide is CD80. CD80 can interact with CD28, CTLA-4, and PD-L1. CD80 is the ligand of CD28 molecule, the main co-stimulatory molecule expressed by T cells. Upon activation, T cells express CTLA-4 and PD-L1, which negatively regulate T-cell receptor (“TCR”) and CD28 downstream signaling. CTLA-4 functions as a CD28 competitor by interacting with CD80 (affinity of interaction of CTLA-4/CD80 is usually higher than CD80/CD28). PD-L1, the receptor of PDL1, functions as T cell inhibitor by de-phosphorylation of TCR and CD28 downstream signalosome. Similar to CTLA-4, PD-L1 is able to interact with CD80 under certain conditions.

The presently disclosed fusion polypeptide (e.g., a fusion polypeptide comprising an extracellular domain and a transmembrane domain of CD80) can ligate three different receptors: CD28, CTLA-4 and PD-L1. By doing so, these ligations provide (i) signaling through the intracellular domain of the co-stimulatory molecule (e.g., 4-1BB, CD28, OX40, ICOS, DAP-10, CD27, CD40, NKG2D, or CD2) and (ii) competes with CTLA4/CD80 and PDL-1/PD-1 ligation, thereby shifting inhibitory signaling to activation signaling to T cells. Constitutive expression of CD80 (e.g., at a high level) on T cell provides a strong co-stimulation upon interaction with endogenous CD28. By interacting with CTLA-4 and PD-L1, fusion polypeptide comprising an extracellular domain and a transmembrane domain of CD80 and can compete with endogenous CD80/CTLA4 and PD-1/PD-L1 (e.g., expressed at a low level) to afford positive signaling into T cells. CTLA-4 is expressed on engineered and bystander T cells. PD-L1 is expressed on different cell types (mainly antigen presenting cells and tumor cells) and can be found on engineered T cells as well. Replacing the intracellular domain of CD80 by co-stimulatory derived signaling domain (e.g., an intracellular domain of a co-stimulatory molecule (e.g., CD28, and/or 4-1BB, etc.) shifts CD80 signaling from negative to positive.

The presently disclosed fusion polypeptide (e.g., a fusion polypeptide comprising an extracellular domain and a transmembrane domain of CD80) provides stimulation of T cells in cis and in trans via engagement with CD28 molecule as well as delivering signaling via the intracellular domain of one or more co-stimulatory molecules (e.g., 4-1BB, and/or CD28). Moreover, a fusion polypeptide comprising an extracellular domain and a transmembrane domain of CD80 can engage with CTLA-4 and PD-L1, two inhibitory molecules are known to be up-regulated in the tumor microenvironment. These interactions shift T cell inhibition into T cells activation. Thus, the presently disclosed fusion polypeptide (e.g., a fusion polypeptide comprising an extracellular domain and a transmembrane domain of CD80) affords co-stimulation into T cells and benefit positively from T cell inhibitory receptors expressed in a hostile tumor microenvironment.

5.2.6. Regulatability of the Fusion Polypeptides

In certain embodiments, the fusion polypeptide can be regulated, e.g., is regulatable. A regulator can regulate or modulate the expression and/or activity of the fusion polypeptide. With the regulator, the expression and/or activity of the fusion polypeptide can be switched on, or switched off. The fusion polypeptide can be regulated by controlling the levels of its expression (i.e., constitutive high, constitutive low or inducible) or by administration of exogenous molecules (e.g., antibodies, Ig fusion proteins). These approaches serve the purpose of switching on and off the function of the fusion polypeptide.

Non-limiting examples of regulators include promoters (e.g., inducible promoters) that capable of controlling the expression of the fusion polypeptide, molecules that are capable of regulating or modulating the expression and/or activity of the co-stimulatory ligand comprised in the fusion polypeptide, and molecules that are capable of regulating or modulating the expression and/or activity of the co-stimulatory molecule comprised in the fusion polypeptide.

Molecules that are capable of regulating or modulating the expression and/or activity of the co-stimulatory ligand comprised in the fusion polypeptide include antibodies binding to the co-stimulatory ligand, and fusion proteins binding to the co-stimulatory ligand and modulating expression and/or activity of the co-stimulatory ligand. In certain embodiment, the regulator is an antibody that binds to the co-stimulatory ligand.

Molecules that are capable of regulating or modulating the expression and/or activity of the co-stimulatory molecule comprised in the fusion polypeptide include antibodies binding to the co-stimulatory molecule, and fusion proteins binding to the co-stimulatory molecule and modulating expression and/or activity of the co-stimulatory molecule. In certain embodiment, the regulator is an antibody that binds to the co-stimulatory molecule.

In certain embodiments, the fusion polypeptide comprises an extracellular domain and a transmembrane domain of CD80, and the regulator is an anti-CD80 antibody.

In certain embodiments, the fusion polypeptide comprises an extracellular domain and a transmembrane domain of CD80, and the regulator is a fusion protein that binds to CD80 and modulates the activity of CD80. In certain embodiments, the fusion protein comprise an CTLA4 fragment that binds to CD80. In certain embodiments, the CTLA4 fragment is selected from the group consisting of Abatacept and belatacept.

5.2.7. Nucleic Acid Molecules Encoding the Fusion Polypeptides

The presently disclosed subject matter further provides nucleic acid molecules encoding the fusion polypeptides disclosed herein. In addition, the presently disclosed subject matter provides vectors comprising the nucleic acid molecules described herein. The vectors can be viral vectors or non-viral vectors. In certain embodiments, the vector is a viral vector. In certain embodiments, the viral vector is a retroviral vector, e.g., a gamma-retroviral vector, or a lentiviral vector.

### 5.3. Cells

The presently disclosed subject matter provides cells comprising a fusion polypeptide disclosed herein. In certain embodiments, the fusion protein is capable of promoting an anti-tumor effect of the cell. In certain embodiments, the cell is selected from the group consisting of cells of lymphoid lineage and cells of myeloid lineage. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell (e.g., embryonic stem cell).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., T_{EM} cells and T_{EMRA} cells, Regulatory T cells (also known as suppressor T cells), tumor-infiltrating lymphocyte (TIL), Natural Killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR. The T cell can be a CD4⁺ T cell or a CD8⁺ T cell. In certain embodiments, the T cell is a CD4⁺ T cell. In certain embodiments, the T cell is a CD8⁺ T cell. In certain embodiments, the CD8⁺ T cell is CD4 independent. In certain embodiments, the T cell is derived from an induced pluripotent stem cell (iPSC). In certain embodiments, the T cell is a CD8⁺ T cell that is CD4 independent, and the CD8⁺ T cell is derived from an iPSC.

In certain embodiments, the cell is a NK cell. Natural Killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells.

In certain embodiments, the presently disclosed cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred CAR modified T cells upon encounter with targeted tumor cells.

In certain embodiments, the cell is a cell of the myeloid lineage. Non-limiting examples of cells of the myeloid lineage include monocytes, macrophages, neutrophils, basophils, eosinophils, erythrocytes, megakaryocytes, and stem cells from which myeloid cells may be differentiated.

In certain embodiments, the stem cell is a pluripotent stem cell (e.g., an embryonic stem cell or an induced pluripotent stem cell).

In certain embodiments, the cell further comprises an antigen-recognizing receptor. In certain embodiments, the antigen-recognizing receptor is capable of activating the cell. The cells can be transduced with an antigen-recognizing receptor and a fusion polypeptide such that the cells co-express the antigen-recognizing receptor and the fusion polypeptide.

The fusion polypeptide can be operably linked to a first promoter. The antigen-recognizing receptor can be operably linked to a second promoter. The first promoter can be the same as the second promoter. Alternatively, the first promoter is different from the second promoter. The first and the second promoters can be endogenous or exogenous. Non-limiting examples of exogenous promoters include an elongation factor (EF)-1 promoter, a cytomegalovirus immediate-early promoter (CMV) promoter, a simian virus 40 early promoter (SV40) promoter, a phosphoglycerate kinase (PGK) promoter, and a metallothionein promoter. In certain embodiments, one or both of the first and second promoters are inducible promoters. In certain embodiment, the inducible promoter is selected from a NFAT transcriptional response element (TRE) promoter, a CD69 promoter, a CD25 promoter, and an IL-2 promoter.

In certain embodiments, the cell is a T cell, and the fusion polypeptide and the antigen-recognizing receptor are integrated at a locus within the genome of the T cell. Non-limiting examples of the loci include a TRAC locus, a TRBC locus, a TRDC locus, and a TRGC locus. In certain embodiments, the locus is a TRAC locus or a TRBC locus. Methods of targeting a CAR to a site within the genome of T cell are disclosed in WO2017180989 and Eyquem et al., Nature. (2017 Mar. 2); 543(7643): 113-117, both of which are incorporated by reference in their entireties.

In certain embodiments, the fusion polypeptide and the antigen-recognizing receptor are integrated at a locus encoding an immune inhibitory molecule. Non-limiting examples of immune inhibitory molecules include CTLA-4, PD-1, LAG3, BTLA, B7-1, B7-H1, B7-H3, B7-H4, TIM3, SHP-1, SHP-2, TIGIT, CD160, and LAIR1.

### 5.4. Antigen-Recognizing Receptors

In certain embodiments, the antigen-recognizing receptor binds to an antigen of interest. In certain embodiments, the antigen is a tumor antigen or a pathogen antigen. In certain embodiments, the antigen-recognizing receptor is a chimeric antigen receptor (CAR). In certain embodiments, the antigen-recognizing receptor is a T-cell receptor (TCR). In certain embodiments, the antigen-recognizing receptor is a TCR like fusion molecule.

5.4.1. Antigens

In certain embodiments, the antigen-recognizing receptor binds to a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. Non-limiting examples of tumor antigens include CD19, carbonic anhydrase IX (CAIX), carcinoembryonic antigen (CEA), CD8, CD7, CD10, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinase Erb-B2, Erb-B3, Erb-B4, folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-VIII, CD99, CD70, ADGRE2, CCR1, LILRB2, PRAME, and ERBB.

In certain embodiments, the antigen-recognizing receptor binds to CD19. In certain embodiments, the antigen-recognizing receptor binds to a murine CD19 polypeptide. In certain embodiments, the murine CD19 polypeptide comprises the amino acid sequence set forth in SEQ ID NO: 33. SEQ ID NO: 33 is provided below.

[SEQ ID NO: 33]
RPQKSLLVEVEEGGNVVLPCLPDSSPVSSEKLAWYRGNQSTPFLELSPG
SPGLGLHVGSLGILLVIVNVSDHMGGFYLCQKRPPFKDIWQPAWTVNVE
DSGEMERWNASDVRDLDCDLRNRSSGSHRSTSGSQLYVWAKDHPKVWGT
KPVCAPRGSSLNQSLINQDLTVAPGSTLWLSCGVPPVPVAKGSISWTHV
HPRRPNVSLLSLSLGGEHPVREMWVWGSLLLLPQATALDEGTYYCLRGN
LTIERHVKVIARSAVWLWLLRTGG

In certain embodiments, the antigen-recognizing receptor binds to a human CD19 polypeptide. In certain embodiments, the human CD19 polypeptide comprises the amino acid sequence set forth in SEQ ID NO: 34. SEQ ID NO: 34 is provided below.

[SEQ ID NO: 34]
PEEPLVVKVEEGDNAVLQCLKGTSDGPTQQLTWSRESPLKPFLKLSLGL
PGLGIHMRPLAIWLFIFNVSQQMGGFYLCQPGPPSEKAWQPGWTVNVEG
SGELFRWNVSDLGGLGCGLKNRSSEGPSSPSGKLMSPKLYVWAKDRPEI
WEGEPPCLPPRDSLNQSLSQDLTMAPGSTLWLSCGVPPDSVSRGPLSWT
HVHPKGPKSLLSLELKDDRPARDMWVMETGLLLPRATAQDAGKYYCHRG
NLTMSFHLEITARPVLWHWLLRTGGWK

In certain embodiments, the antigen-recognizing receptor binds to the extracellular domain of a CD19 protein.

In certain embodiments, the antigen-recognizing receptor binds to a pathogen antigen, e.g., for use in treating and/or preventing a pathogen infection or other infectious disease, for example, in an immunocompromised subject. Non-limiting examples of pathogen includes a virus, bacteria, fungi, parasite and protozoa capable of causing disease.

Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella, Legionella pneumophilia, Mycobacteria sps (e.g. M. tuberculosis, M. avium, M. intracellulare, M. kansaii, M. gordonae, M. leprae), Staphylococcus aureus, Staphylococcus epidermidis, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Campylobacter jejuni, Enterococcus sp., Haemophilus influenzae, Bacillus antracis, Corynebacterium diphtheriae, Corynebacterium sp., Erysipelothrix rhusiopathiae, Clostridium spp., Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli. Mycoplasma, Pseudomonas aeruginosa, Pseudomonas fluorescens, Corynobacteria diphtheriae, Bartonella henselae, Bartonella quintana, Coxiella burnetii, chlamydia, shigella, Yersinia enterocolitica, Yersinia pseudotuberculosis, Listeria monocytogenes, Mycoplasma spp., Vibrio cholerae, Borrelia, Francisella, Brucella melitensis, Proteus mirabilis, and Proteus.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

5.4.2. T-Cell Receptor (TCR)

In certain embodiments, the antigen-recognizing receptor is a TCR. A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The Variable region binds to the peptide/MHC complex. The variable domain of both chains each has three complementarity determining regions (CDRs).

In certain embodiments, a TCR can form a receptor complex with three dimeric signaling modules CD3δ/ε, CD3γ/ε and CD247 ζ/ζ or ζ/η. When a TCR complex engages with its antigen and MEW (peptide/MHC), the T cell expressing the TCR complex is activated.

In certain embodiments, the antigen-recognizing receptor is an endogenous TCR. In certain embodiments, the antigen-recognizing receptor is naturally occurring TCR.

In certain embodiments, the antigen-recognizing receptor is an exogenous TCR. In certain embodiments, the antigen-recognizing receptor is a recombinant TCR. In certain embodiments, the antigen-recognizing receptor is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

5.4.3. Chimeric Antigen Receptor (CAR)

In certain embodiments, the antigen-recognizing receptor is a chimeric antigen receptor (CAR). CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., a scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40, CD27, CD40 and NKG2D) to the cytoplasmic tail of the CAR to provide additional signals to the T cell. “Second generation” CARs comprise those that provide both co-stimulation (e.g., CD28 or 4-1BB) and activation (CD3). “Third generation” CARs comprise those that provide multiple co-stimulation (e.g., CD28 and 4-1BB) and activation (CD3ζ). In certain embodiments, the CAR is a second-generation CAR. In certain embodiments, the CAR comprises an extracellular antigen-binding domain that binds to an antigen, a transmembrane domain, and an intracellular signaling domain, wherein the intracellular signaling domain comprises a co-stimulatory signaling domain. In certain embodiments, the CAR further comprises a hinger/spacer region.

In certain non-limiting embodiments, the extracellular antigen-binding domain of the CAR (for example, a scFv or an analog thereof) binds to an antigen with a dissociation constant (K_{d}) of about 2×10⁻⁷ M or less. In certain embodiments, the K_{d} is about 2×10⁻⁷ M or less, about 1×10⁻⁷ M or less, about 9×10⁻⁸ M or less, about 1×10⁻⁸ M or less, about 9×10⁻⁹ M or less, about 5×10⁻⁹ M or less, about 4×10⁻⁹ M or less, about 3×10⁻⁹ or less, about 2×10⁻⁹ M or less, about 1×10⁻⁹ M or less, about 1×10⁻¹⁰ M or less, or about 1×10⁻¹¹M or less. In certain non-limiting embodiments, the K_{d} is about 1×10⁻⁸M or less. In certain non-limiting embodiments, the K_{d} is about 1×10⁻⁹M or less. In certain non-limiting embodiments, the K_{d} is from about 1×10⁻⁹M to about 1×10⁻⁷ M.

Binding of the extracellular antigen-binding domain (for example, in a scFv or an analog thereof) can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (MA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet).

5.4.3.1. Extracellular Antigen Binding Domain of a CAR

In certain embodiments, the extracellular antigen-binding domain specifically binds to an antigen. In certain embodiments, the extracellular antigen-binding domain is a scFv. In certain embodiments, the scFv is a human scFv. In certain embodiments, the scFv is a humanized scFv. In certain embodiments, the scFv is a murine scFv. In certain embodiments, the extracellular antigen-binding domain is a Fab, which is optionally crosslinked. In certain embodiments, the extracellular antigen-binding domain is a F(ab)₂. In certain embodiments, any of the foregoing molecules may be comprised in a fusion protein with a heterologous sequence to form the extracellular antigen-binding domain. In certain embodiments, the scFv is identified by screening scFv phage library with an antigen-Fc fusion protein. The scFv can be derived from a mouse bearing human V_{L} and/or V_{H} genes. The scFv can also be substituted with a camelid Heavy chain (e.g., VHH, from camel, lama, etc.) or a partial natural ligand for a cell surface receptor. In certain embodiments, the antigen is a tumor antigen, e.g., one disclosed herein. In certain embodiments, the antigen is a pathogen antigen, e.g., one disclosed herein.

In certain embodiments, the extracellular antigen-binding domain is a murine scFv. In certain embodiments, the extracellular antigen-binding domain is a murine scFv that binds to a human CD19 polypeptide. In certain embodiments, the extracellular antigen-binding domain comprises the amino acid sequence set forth in SEQ ID NO: 35 and specifically binds to a human CD19 polypeptide (e.g., a human CD19 polypeptide comprising the amino acid sequence set forth in SEQ ID NO: 34 or a portion thereof). In certain embodiments, the nucleotide sequence encoding the amino acid sequence of SEQ ID NO: 35 is set forth in SEQ ID NO: 36. In certain embodiments, the scFv is from a clone designated as “SJ25C1”.

In certain embodiments, the extracellular antigen-binding domain comprises a heavy chain variable region (V_{H}) CDR1 comprising the amino acid sequence set forth in SEQ ID NO: 37 or a conservative modification thereof, a V_{H} CDR2 comprising the amino acid sequence set forth in SEQ ID NO: 38 or a conservative modification thereof, and a V_{H} CDR3 comprising the amino acid sequence set forth in SEQ ID NO: 39 or a conservative modification thereof. In certain embodiments, the extracellular antigen-binding domain comprises a V_{H} CDR1 comprising the amino acid sequence set forth in SEQ ID NO: 37, a V_{H} CDR2 comprising the amino acid sequence set forth in SEQ ID NO: 38, and a V_{H} CDR3 comprising the amino acid sequence set forth in SEQ ID NO: 39. In certain embodiments, the extracellular antigen-binding domain comprises a light chain variable region (V_{L}) CDR1 comprising the amino acid sequence set forth in SEQ ID NO: 40 or a conservative modification thereof, a V_{L} CDR2 comprising the amino acid sequence set forth in SEQ ID NO: 41 or a conservative modification thereof, and a V_{L} CDR3 comprising the amino acid sequence set forth in SEQ ID NO: 42 or a conservative modification thereof. In certain embodiments, the extracellular antigen-binding domain comprises a V_{L} CDR1 comprising the amino acid sequence set forth in SEQ ID NO: 40, a V_{L} CDR2 comprising the amino acid sequence set forth in SEQ ID NO: 41, and a V_{L} CDR3 comprising the amino acid sequence set forth in SEQ ID NO: 42.

In certain embodiments, the extracellular antigen-binding domain comprises a V_{H} CDR1 comprising the amino acid sequence set forth in SEQ ID NO: 37 or a conservative modification thereof, a V_{H} CDR2 comprising the amino acid sequence set forth in SEQ ID NO: 38 or a conservative modification thereof, a V_{H} CDR3 comprising the amino acid sequence set forth in SEQ ID NO: 39, a conservative modification thereof, a V_{L} CDR1 comprising the amino acid sequence set forth in SEQ ID NO: 40 or a conservative modification thereof, a V_{L} CDR2 comprising the amino acid sequence set forth in SEQ ID NO: 41 or a conservative modification thereof, and a V_{L} CDR3 comprising the amino acid sequence set forth in SEQ ID NO: 42 or a conservative modification thereof. In certain embodiments, the extracellular antigen-binding domain comprises a V_{H} CDR1 comprising amino acids having the sequence set forth in SEQ ID NO: 37, a V_{H} CDR2 comprising the amino acid sequence set forth in SEQ ID NO: 38, a V_{H} CDR3 comprising the amino acid sequence set forth in SEQ ID NO: 39, a V_{L} CDR1 comprising the amino acid sequence set forth in SEQ ID NO: 40, a V_{L} CDR2 comprising the amino acid sequence set forth in SEQ ID NO: 41, and a V_{L} CDR3 comprising the amino acid sequence set forth in SEQ ID NO: 42.

In certain embodiments, the extracellular antigen-binding domain comprises a V_{H} comprising an amino acid sequence that is at least about 80% (e.g., at least about 85%, at least about 90%, or at least about 95%) homologous or identical to the amino acid sequence set forth in SEQ ID NO: 43. For example, the extracellular antigen-binding domain comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 43. In certain embodiments, the extracellular antigen-binding domain comprises a V_{H} comprising the amino acid sequence set forth in SEQ ID NO: 43.

In certain embodiments, the extracellular antigen-binding domain comprises a V_{L} comprising an amino acid sequence that is at least about 80% (e.g., at least about 85%, at least about 90%, or at least about 95%) homologous or identical to the amino acid sequence set forth in SEQ ID NO: 44. For example, the extracellular antigen-binding domain comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 44. In certain embodiments, the extracellular antigen-binding domain comprises a V_{L} comprising the amino acid sequence set forth in SEQ ID NO: 44.

In certain embodiments, the extracellular antigen-binding domain comprises a V_{H} comprising an amino acid sequence that is at least about 80% (e.g., at least about 85%, at least about 90%, or at least about 95%) homologous or identical to the amino acid sequence set forth in SEQ ID NO: 43, and a V_{L} comprising an amino acid sequence that is at least about 80% (e.g., at least about 85%, at least about 90%, or at least about 95%) homologous or identical to the amino acid sequence set forth in SEQ ID NO: 44.

In certain embodiments, the extracellular antigen-binding domain comprises a V_{H} comprising the amino acid sequence set forth in SEQ ID NO: 43. In certain embodiments, the extracellular antigen-binding domain comprises a V_{L} comprising the amino acid sequence set forth in SEQ ID NO: 44. In certain embodiments, the extracellular antigen-binding domain comprises V_{H} comprising the amino acid sequence set forth in SEQ ID NO: 43 and a V_{L} comprising the amino acid sequence set forth in SEQ ID NO: 44, optionally with (iii) a linker sequence, for example a linker peptide, between the V_{H} and the V_{L}. In certain embodiments, the linker comprises the amino acid sequence set forth in SEQ ID NO: 7.

SEQ ID NOs: 35-44 are provided in the following Table 1.

TABLE 1

| | | anti-human CD19 scFv | | |
|---|---|---|---|---|
| CDRs | | 1 | 2 | 3 |
| V_{H} | a.a. | GYAFSS [SEQ ID | YPGDGD [SEQ ID NO: | KTISSVVDF [SEQ ID |
| | | NO: 37] | 38] | NO: 39] |
| V_{L} | a.a. | KASQNVGTNVA [SEQ | SATYRN [SEQ ID NO: | QQYNRYPYT [SEQ ID |
| | | ID NO: 40] | 41] | NO: 42] |

| | |
|---|---|
| Full V_{H} | EVKLQQSGAE LVRPGSSVKI SCKASGYAFS SYWMNWVKQR PGQGLEWIGQ |
| | IYPGDGDTNY NGKFKGQATL TADKSSSTAY MQLSGLTSED SAVYFCARKT |
| | ISSVVDFYFD YWGQGTTVTV SS [SEQ ID NO: 43] |
| Full V_{L} | DIELTQSPKF MSTSVGDRVS VTCKASQNVG TNVAWYQQKP GQSPKPLIYS |
| | ATYRNSGVPD RFTGSGSGTD FTLTITNVQS KDLADYFCQQ YNRYPYTSGG |
| | GTKLEIKR [SEQ ID NO: 44] |
| scFv | MALPVTALLL PLALLLHAEV KLQQSGAELV RPGSSVKISC KASGYAFSSY |
| | WMNWVKQRPG QGLEWIGQIY PGDGDTNYNG KFKGQATLTA DKSSSTAYMQ |
| | LSGLTSEDSA VYFCARKTIS SVVDFYFDYW GQGTTVTVSS GGGGSGGGGS |
| | GGGGSDIELT QSPKFMSTSV GDRVSVTCKA SQNVGTNVAW YQQKPGQSPK |
| | PLIYSATYRN SGVPDRFTGS GSGTDFTLTI TNVQSKDLAD YFCQQYNRYP |
| | YTSGGGTKLE IKR [SEQ ID NO: 35] |
| DNA | ATGGCTCTCCCAGTGACTGCCCTACTGCTTCCCCTAGCGCTTCTCCTGCATGCAGAGG |
| | TGAAGCTGCAGCAGTCTGGGGCTGAGCTGGTGAGGCCTGGGTCCTCAGTGAAGATTTC |
| | CTGCAAGGCTTCTGGCTATGCATTCAGTAGCTACTGGATGAACTGGGTGAAGCAGAGG |
| | CCTGGACAGGGTCTTGAGTGGATTGGACAGATTTATCCTGGAGATGGTGATACTAACT |
| | ACAATGGAAAGTTCAAGGGTCAAGCCACACTGACTGCAGACAAATCCTCCAGCACAGC |
| | CTACATGCAGCTCAGCGGCCTAACATCTGAGGACTCTGCGGTCTATTTCTGTGCAAGA |
| | AAGACCATTAGTTCGGTAGTAGATTTCTACTTTGACTACTGGGGCCAAGGGACCACGG |
| | TCACCGTCTCCTCAGGTGGAGGTGGATCAGGTGGAGGTGGATCTGGTGGAGGTGGATC |
| | TGACATTGAGCTCACCCAGTCTCCAAAATTCATGTCCACATCAGTAGGAGACAGGGTC |
| | AGCGTCACCTGCAAGGCCAGTCAGAATGTGGGTACTAATGTAGCCTGGTATCAACAGA |
| | AACCAGGACAATCTCCTAAACCACTGATTTACTCGGCAACCTACCGGAACAGTGGAGT |
| | CCCTGATCGCTTCACAGGCAGTGGATCTGGGACAGATTTCACTCTCACCATCACTAAC |
| | GTGCAGTCTAAAGACTTGGCAGACTATTTCTGTCAACAATATAACAGGTATCCGTACA |
| | CGTCCGGAGGGGGGACCAAGCTGGAGATCAAACGG [SEQ ID NO: 36] |

As used herein, the term “a conservative sequence modification” refers to an amino acid modification that does not significantly affect or alter the binding characteristics of the presently disclosed CAR (e.g., the extracellular antigen-binding domain of the CAR) comprising the amino acid sequence. Conservative modifications can include amino acid substitutions, additions and deletions. Modifications can be introduced into the human scFv of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

The V_{H} and/or V_{L} amino acid sequences having at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NOs: 43 and 44) may contain substitutions (e.g., conservative substitutions), insertions, or deletions relative to the specified sequence(s), but retain the ability to bind to a target antigen (e.g., CD19). In certain embodiments, a total of 1 to 10 amino acids are substituted, inserted and/or deleted in a specific sequence (e.g., SEQ ID NOs: 43 and 44). In certain embodiments, substitutions, insertions, or deletions occur in regions outside the CDRs (e.g., in the FRs) of the extracellular antigen-binding domain. In certain embodiments, the extracellular antigen-binding domain comprises V_{H} and/or V_{L}, sequence selected from SEQ ID NOs: 43 and 44, including post-translational modifications of that sequence (SEQ ID NO: 43 and 44).

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the)(BLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

5.4.3.2. Transmembrane Domain of a CAR

In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain of the CAR can comprise a native or modified transmembrane domain of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 peptide, a NKG2D peptide, a synthetic polypeptide (not based on a protein associated with the immune response), or a combination thereof.

CD8

In certain embodiments, the transmembrane domain comprises a CD8 polypeptide. In certain embodiments, the CD8 polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the amino acid sequence having a NCBI Reference No: NP_001139345.1 (SEQ ID NO: 45) (homology herein may be determined using standard software such as BLAST or FASTA) as provided below, or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 45, which is at least 20, or at least 30, or at least 40, or at least 50, and up to 235 amino acids in length. Alternatively or additionally, in non-limiting various embodiments, the CD8 polypeptide comprises or consists of an amino acid sequence of amino acids 1 to 235, 1 to 50, 50 to 100, 100 to 150, 150 to 200, or 200 to 235 of SEQ ID NO: 45. In certain embodiments, the transmembrane domain of the CAR comprises a CD8 polypeptide that comprises or consists of amino acids 137 to 209 of SEQ ID NO: 45. SEQ ID NO: 45 is provided below.

[SEQ ID NO: 45]
MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV

In certain embodiments, the CD8 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence having a GenBank No: AAA92533.1 (SEQ ID NO: 46) (homology herein may be determined using standard software such as BLAST or FASTA) as provided below, or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 46, which is at least about 20, or at least about 30, or at least about 40, or at least about 50, or at least about 60, or at least about 70, or at least about 100, or at least about 200, and up to 247 amino acids in length. Alternatively or additionally, in non-limiting various embodiments, the CD8 polypeptide comprises or consists of amino acids 1 to 247, 1 to 50, 50 to 100, 100 to 150, 150 to 200, 151 to 219, or 200 to 247 of SEQ ID NO: 46. In certain embodiments, the transmembrane domain of the CAR comprises a CD8 polypeptide that comprises or consists of amino acids 151 to 219 of SEQ ID NO: 46. SEQ ID NO: 46 is provided below.

| [SEQ ID NO: 46] | |
|---|---|
| 1 | MASPLTRELS LNLLLMGESI ILGSGEAKPQ APELRIFPKK MDAELGQKVD LVCEVLGSVS |
| 61 | QGCSWLFQNS SSKLPQPTFV VYMASSHNKI TWDEKLNSSK LFSAVRDTNN KYVLTLNKFS |
| 121 | KENEGYYFCS VISNSVMYFS SVVPVLQKVN STTTKPVLRT PSPVHPTGTS QPQRPEDCRP |
| 181 | RGSVKGTGLD FACDIYIWAP LAGICVAPLL SLIITLICYH RSRKRVCKCP RPLVRQEGKP |
| 241 | RPSEKIV |

In certain embodiments, the CD8 polypeptide comprises or consists of the amino acid sequence set forth in SEQ ID NO: 47, which is provided below:

[SEQ ID NO: 47]
STTTKPVLRTPSPVHPTGTSQPQRPEDCRPRGSVKGTGLDFACDIYIWAP
LAGICVALLLSLITTLICY

In accordance with the presently disclosed subject matter, a “CD8 nucleic acid molecule” refers to a polynucleotide encoding a CD8 polypeptide.

An exemplary nucleic acid sequence encoding the amino acid sequence of SEQ ID NO: 47 comprises or consists of the nucleotide sequence set forth in SEQ ID NO: 48, which is provided below.

[SEQ ID NO: 48]
TCTACTACTACCAAGCCAGTGCTGCGAACTCCCTCACCTGTGCACCCTA
CCGGGACATCTCAGCCCCAGAGACCAGAAGATTGTCGGCCCCGTGGCTC
AGTGAAGGGGACCGGATTGGACTTCGCCTGTGATATTTACATCTGGGCA
CCCTTGGCCGGAATCTGCGTGGCCCTTCTGCTGTCCTTGATCATCACTC
TCATCTGCTAC

CD28

In certain embodiments, the transmembrane domain of a presently disclosed CAR comprises a CD28 polypeptide. In certain embodiments, the CD28 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 25 or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 25, which is at least 20, or at least 30, or at least 40, or at least 50, and up to 220 amino acids in length. Alternatively or additionally, in non-limiting various embodiments, the CD28 polypeptide comprises or consists of an amino acid sequence of amino acids 1 to 220, 1 to 50, 50 to 100, 100 to 150, 114 to 220, 150 to 200, 153 to 179, or 200 to 220 of SEQ ID NO: 25. In certain embodiments, the CAR comprises a transmembrane domain of CD28 (e.g., human CD28) or a portion thereof. In certain embodiments, the transmembrane domain of CD28 or a portion thereof comprises or consists of amino acids 153 to 179 of SEQ ID NO: 25. In certain embodiments, the CAR comprises a CD28 polypeptide comprising or consisting of amino acids 153 to 179 of SEQ ID NO: 25.

An exemplary nucleic acid sequence encoding amino acids 153 to 179 of SEQ ID NO: 25 comprises or consists of the nucleotide sequence set forth in SEQ ID NO: 49, which is provided below.

[SEQ ID NO: 49]
ttttgggtgctggtggtggttggtggagtcctggcttgctatagcttgc
tagtaacagtggcctttattattttctgggtg

CD84

In certain embodiments, the transmembrane domain of a presently disclosed CAR comprises a native or modified transmembrane domain of a CD84 polypeptide. In certain embodiments, the CD84 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence having a NCBI Reference No: NP_001171808.1 (SEQ ID No: 50), or a fragment thereof, and/or may optionally comprise up to one or up to two or up to three conservative amino acid substitutions. In non-limiting certain embodiments, the CD84 polypeptide comprises or consists of an amino acid sequence that is a consecutive portion of SEQ ID NO: 50, which is at least 20, or at least 30, or at least 40, or at least 50, and up to 345 amino acids in length. Alternatively or additionally, in non-limiting various embodiments, the CD84 polypeptide comprises or consists of an amino acid sequence of amino acids 1 to 345, 1 to 50, 50 to 100, 100 to 150, 150 to 200, 200 to 250, 226 to 250, 250 to 300, or 300 to 345 of SEQ ID NO: 50. In certain embodiments, the transmembrane domain of the CAR comprises a CD84 polypeptide comprising or consisting of amino acids 226 to 250 of SEQ ID NO: 50.

SEQ ID NO: 50 is provided below:

| [SEQ ID NO: 50] | |
|---|---|
| 1 | MAQHHLWILL LCLQTWPEAA GKDSEIFTVN GILGESVTFP VNIQEPRQVK IIAWTSKTSV |
| 61 | AYVTPGDSET APVVTVTHRN YYERIHALGP NYNLVISDLR MEDAGDYKAD INTQADPYTT |
| 121 | TKRYNLQIYR RLGKPKITQS LMASVNSTCN VTLTCSVEKE EKNVTYNWSP LGEEGNVLQI |
| 181 | FQTPEDQELT YTCTAQNPVS NNSDSISARQ LCADIAMGFR THHTGLLSVL AMFFLLVLIL |
| 241 | SSVFLFRLFK RRQGRIFPEG SCLNTFTKNP YAASKKTIYT YIMASRNTQP AESRIYDEIL |
| 301 | QSKVLPSKEE PVNTVYSEVQ FADKMGKAST QDSKPPGTSS YEIVI |

In accordance with the presently disclosed subject matter, a “CD84 nucleic acid molecule” refers to a polynucleotide encoding a CD84 polypeptide. An exemplary nucleic acid sequence encoding amino acids 226 to 250 of SEQ ID NO: 50 comprises or consists of the nucleotide sequence set forth in SEQ ID NO: 51, which is provided below.

[SEQ ID NO: 51]
TTGCTGAGCGTGCTGGCTATGTTCTTTCTGCTTGTTCTCATTCTGTCTT
CAGTGTTTTTGTTCCGTTTGTTCAAG

CD166

In certain embodiments, the transmembrane domain of a presently disclosed CAR comprises a native or modified transmembrane domain of a CD166 polypeptide. In certain embodiments, the CD166 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence having a NCBI Reference No: NP_001618.2 (SEQ ID NO: 52), or a fragment thereof, and/or may optionally comprise up to one or up to two or up to three conservative amino acid substitutions. In non-limiting certain embodiments, the CD166 polypeptide comprises or consists of an amino acid sequence that is a consecutive portion of SEQ ID NO: 52, which is at least 20, or at least 30, or at least 40, or at least 50, and up to 583 amino acids in length. Alternatively or additionally, in non-limiting various embodiments, the CD166 polypeptide comprises or consists of an amino acid sequence of amino acids 1 to 583, 1 to 50, 50 to 100, 100 to 150, 150 to 200, 200 to 250, 250 to 300, 300 to 350, 350 to 400, 400 to 450, 450 to 500, 500 to 550, 528 to 549, 528 to 553, or 550 to 583 of SEQ ID NO: 52. In certain embodiments, the transmembrane domain of the CAR comprises a CD166 polypeptide comprising or consisting of amino acids 528 to 553 of SEQ ID NO: 52. In certain embodiments, the transmembrane domain the CAR comprises a CD166 polypeptide comprising or consisting of amino acids 528 to 549 of SEQ ID NO: 52.

SEQ ID NO: 52 is provided below:

| [SEQ ID NO: 52] | |
|---|---|
| 1 | MESKGASSCR LLFCLLISAT VFRPGLGWYT VNSAYGDTII IPCRLDVPQN LMFGKWKYEK |
| 61 | PDGSPVFIAF RSSTKKSVQY DDVPEYKDRL NLSENYTLSI SNARISDEKR FVCMLVTEDN |
| 121 | VFEAPTIVKV FKQPSKPEIV SKALFLETEQ LKKLGDCISE DSYPDGNITW YRNGKVLHPL |
| 181 | EGAVVIIFKK EMDPVTQLYT MTSTLEYKTT KADIQMPFTC SVTYYGPSGQ KTIHSEQAVF |
| 241 | DIYYPTEQVT IQVLPPKNAI KEGDNITLKC LGNGNPPPEE FLFYLPGQPE GIRSSNTYTL |
| 301 | TDVRRNATGD YKCSLIDKKS MIASTAITVH YLDLSLNPSG EVTRQIGDAL PVSCTISASR |
| 361 | NATVVWMKDN IRLRSSPSFS SLHYQDAGNY VCETALQEVE GLKKRESLTL IVEGKPQIKM |
| 421 | TKKTDPSGLS KTIICHVEGF PKPAIQWTIT GSGSVINQTE ESPYINGRYY SKIIISPEEN |
| 481 | VTLTCTAENQ LERTVNSLNV SAISIPEHDE ADEISDENRE KVNDQAKLIV GIVVGLLLAA |
| 541 | LVAGVVYWLY MKKSKTASKH VNKDLGNMEE NKKLEENNHK TEA |

In accordance with the presently disclosed subject matter, a “CD166 nucleic acid molecule” refers to a polynucleotide encoding a CD166 polypeptide. An exemplary nucleic acid sequence encoding amino acids 528 to 553 of SEQ ID NO: 52 comprises or consists of the nucleotide sequence set forth in SEQ ID NO: 53, which is provided below.

[SEQ ID NO: 53]
CTAATTGTGGGAATCGTTGTTGGTCTCCTCCTTGCTGCCCTTGTTGCT
GGTGTCGTCTACTGGCTGTACATGAAGAAG

CD8a

In certain embodiments, the transmembrane domain of a presently disclosed CAR comprises a native or modified transmembrane domain of a CD8a polypeptide. In certain embodiments, the CD8a polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence having a NCBI Reference No: NP_001139345.1 (SEQ ID No: 54), or a fragment thereof, and/or may optionally comprise up to one or up to two or up to three conservative amino acid substitutions. In certain embodiments, the CD8a polypeptide comprises or consists of an amino acid sequence that is a consecutive portion of SEQ ID NO: 54, which is at least 20, or at least 30, or at least 40, or at least 50, and up to 235 amino acids in length. Alternatively or additionally, in non-limiting various embodiments, the CD8a polypeptide comprises or consists of an amino acid sequence of amino acids 1 to 235, 1 to 50, 50 to 100, 100 to 150, 150 to 200, 183 to 207, or 200 to 235 of SEQ ID NO: 54. In certain embodiments, the transmembrane domain of the CAR comprises a CD8a polypeptide comprising or consisting of amino acids 183 to 207 of SEQ ID NO: 54.

SEQ ID NO: 54 is provided below:

| [SEQ ID NO: 54] | |
|---|---|
| 1 | MALPVTALLL PLALLLHAAR PSQFRVSPLD RTWNLGETVE LKCQVLLSNP TSGCSWLFQP |
| 61 | RGAAASPTFL LYLSQNKPKA AEGLDTQRFS GKRLGDTFVL TLSDFRRENE GYYFCSALSN |
| 121 | SIMYFSHFVP VFLPAKPTTT PAPRPPTPAP TIASQPLSLR PEACRPAAGG AVHTRGLDFA |
| 181 | CDIYIWAPLA GTCGVLLLSL VITLYCNHRN RRRVCKCPRP VVKSGDKPSL SARYV |

In accordance with the presently disclosed subject matter, a “CD8a nucleic acid molecule” refers to a polynucleotide encoding a CD8a polypeptide. An exemplary nucleic acid sequence encoding amino acids 183 to 207 of SEQ ID NO: 54 comprises or consists of the nucleotide sequence set forth in SEQ ID NO: 55, which is provided below.

[SEQ ID NO: 55]
atctacatctgggcgcccttggccgggacttgtggggtccttctcctg
tcactggttatcaccctttactgcaac

CD8b

In certain embodiments, the transmembrane domain of a presently disclosed CAR comprises a native or modified transmembrane domain of a CD8b polypeptide. In certain embodiments, the CD8b polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence having a NCBI Reference No: NP_742099.1 (SEQ ID No: 56), or a fragment thereof, and/or may optionally comprise up to one or up to two or up to three conservative amino acid substitutions. In certain embodiments, the CD8b polypeptide comprises or consists of an amino acid sequence that is a consecutive portion of SEQ ID NO: 56, which is at least 20, or at least 30, or at least 40, or at least 50, and up to 221 amino acids in length. Alternatively or additionally, in non-limiting various embodiments, the CD8b polypeptide comprises or consists of an amino acid sequence of amino acids 1 to 221, 1 to 50, 50 to 100, 100 to 150, 114 to 220, 150 to 200, 171 to 195, or 200 to 221 of SEQ ID NO: 56. In certain embodiments, the transmembrane domain of the CAR comprises a CD8b polypeptide comprising or consisting of amino acids 171 to 195 of SEQ ID NO: 56.

SEQ ID NO: 56 is provided below:

| [SEQ ID NO: 56] | |
|---|---|
| 1 | MRPRLWLLLA AQLTVLHGNS VLQQTPAYIK VQTNKMVMLS CEAKISLSNM RIYWLRQRQA |
| 61 | PSSDSHHEFL ALWDSAKGTI HGEEVEQEKI AVFRDASRFI LNLTSVKPED SGIYFCMIVG |
| 121 | SPELTFGKGT QLSVVDFLPT TAQPTKKSTL KKRVCRLPRP ETQKGPLCSP ITLGLLVAGV |
| 181 | LVLLVSLGVA IHLCCRRRRA RLRFMKQLRL HPLEKCSRMD Y |

In accordance with the presently disclosed subject matter, a “CD8b nucleic acid molecule” refers to a polynucleotide encoding a CD8b polypeptide. An exemplary nucleic acid sequence encoding amino acids 171 to 195 of SEQ ID NO: 56 comprises or consists of the nucleotide sequence set forth in SEQ ID NO: 57, which is provided below.

[SEQ ID NO: 57]
ATCACCCTTGGCCTGCTGGTGGCTGGCGTCCTGGTTCTGCTGGTTTCCC
TGGGAGTGGCCATCCACCTGTGCTGC

ICOS

In certain embodiments, the transmembrane domain of a presently disclosed CAR comprises a native or modified transmembrane domain of an ICOS polypeptide. In certain embodiments, the ICOS polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID No: 27 or a fragment thereof, and/or may optionally comprise up to one or up to two or up to three conservative amino acid substitutions. In non-limiting certain embodiments, the ICOS polypeptide comprises or consists of an amino acid sequence that is a consecutive portion of SEQ ID NO: 27, which is at least 20, or at least 30, or at least 40, or at least 50, and up to 199 amino acids in length. Alternatively or additionally, in non-limiting various embodiments, the ICOS polypeptide comprises or consists of an amino acid sequence of amino acids 1 to 199, 1 to 50, 50 to 100, 100 to 150, 141 to 165, or 150 to 199 of SEQ ID NO: 27. In certain embodiments, the transmembrane domain of the CAR comprises an ICOS polypeptide comprising or consisting of amino acids 141 to 165 of SEQ ID NO: 27.

In accordance with the presently disclosed subject matter, an “ICOS nucleic acid molecule” refers to a polynucleotide encoding an ICOS polypeptide. An exemplary nucleic acid sequence encoding amino acids 141 to 165 of SEQ ID NO: 27 comprises or consists of the nucleotide sequence set forth in SEQ ID NO: 58, which is provided below.

[SEQ ID NO: 58]
TTCTGGTTACCCATAGGATGTGCAGCCTTTGTTGTAGTCTGCATTTTGGG
ATGCATACTTATTTGTTGGCTTACA

CTLA-4

In certain embodiments, the transmembrane domain of a presently disclosed CAR comprises a native or modified transmembrane domain of a CTLA-4 polypeptide. In certain embodiments, the CTLA-4 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence having a NCBI Reference No: NP_005205.2 (SEQ ID NO: 59), or a fragment thereof, and/or may optionally comprise up to one or up to two or up to three conservative amino acid substitutions. In certain embodiments, the CTLA-4 polypeptide comprises or has an amino acid sequence that is a consecutive portion of SEQ ID NO: 59, which is at least 20, or at least 30, or at least 40, or at least 50, and up to 223 amino acids in length. Alternatively or additionally, in non-limiting various embodiments, the CTLA-4 polypeptide comprises or consists of amino acids 1 to 223, 1 to 50, 50 to 100, 100 to 150, 150 to 200, 162 to 186, or 200 to 223 of SEQ ID NO: 59. In certain embodiments, the transmembrane domain of the CAR comprises a CTLA-4 polypeptide comprising or consisting of amino acids 162 to 186 of SEQ ID NO: 59.

SEQ ID NO: 59 is provided below:

| [SEQ ID NO: 59] | |
|---|---|
| 1 | MACLGFQRHK AQLNLATRTW PCTLLFFLLF IPVFCKAMHV AQPAVVLASS RGIASFVCEY |
| 61 | ASPGKATEVR VTVLRQADSQ VTEVCAATYM MGNELTFLDD SICTGTSSGN QVNLTIQGLR |
| 121 | AMDTGLYICK VELMYPPPYY LGIGNGTQIY VIDPEPCPDS DFLLWILAAV SSGLFFYSFL |
| 181 | LTAVSLSKML KKRSPLTTGV YVKMPPTEPE CEKQFQPYFI PIN |

In accordance with the presently disclosed subject matter, a “CTLA-4 nucleic acid molecule” refers to a polynucleotide encoding a CTLA-4 polypeptide. An exemplary nucleic acid sequence encoding amino acids 162 to 186 of SEQ ID NO: 59 comprises or consists of the nucleotide sequence set forth in SEQ ID NO: 60, which is provided below.

[SEQ ID NO: 60]
TTCCTCCTCTGGATCCTTGCAGCAGTTAGTTCGGGGTTGTTTTTTTATAG
CTTTCTCCTCACAGCTGTTTCTTTG

ICAM-1

In certain embodiments, the transmembrane domain of a presently disclosed CAR comprises a native or modified transmembrane domain of an ICAM-1 polypeptide. In certain embodiments, the ICAM-1 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence having a NCBI Reference No: NP_000192.2 (SEQ ID No: 61), or a fragment thereof, and/or may optionally comprise up to one or up to two or up to three conservative amino acid substitutions. In certain embodiments, the ICAM-1 polypeptide comprises or consists of an amino acid sequence that is a consecutive portion of SEQ ID NO: 61, which is at least 20, or at least 30, or at least 40, or at least 50, and up to 532 amino acids in length. Alternatively or additionally, in non-limiting various embodiments, the ICAM-1 polypeptide comprises or consists of an amino acid sequence of amino acids 1 to 532, 1 to 50, 50 to 100, 100 to 150, 150 to 200, 200 to 225, 250 to 300, 300 to 350, 350 to 400, 400 to 450, 481 to 507, 450 to 500, or 500 to 532 of SEQ ID NO: 61. In certain embodiments, the e transmembrane domain of the CAR comprises an ICAM-1 polypeptide comprising or consisting of amino acids 481 to 507 of SEQ ID NO: 61.

SEQ ID NO: 61 is provided below:

[SEQ ID NO: 61]

| | |
|---|---|
| 1 | MAPSSPRPAL PALLVLLGAL FPGPGNAQTS VSPSKVILPR |
| | GGSVLVTCST SCDQPKLLGI |
| 61 | ETPLPKKELL LPGNNRKVYE LSNVQEDSQP MCYSNCPDGQ |
| | STAKTFLTVY WTPERVELAP |
| 121 | LPSWQPVGKN LTLRCQVEGG APRANLTVVL LRGEKELKRE |
| | PAVGEPAEVT TTVLVRRDHH |
| 181 | GANFSCRTEL DLRPQGLELF ENTSAPYQLQ TFVLPATPPQ |
| | LVSPRVLEVD TQGTVVCSLD |
| 241 | GLFPVSEAQV HLALGDQRLN PTVTYGNDSF SAKASVSVTA |
| | EDEGTQRLTC AVILGNQSQE |
| 301 | TLQTVTIYSF PAPNVILTKP EVSEGTEVTV KCEAHPRAKV |
| | TLNGVPAQPL GPRAQLLLKA |
| 361 | TPEDNGRSFS CSATLEVAGQ LIHKNQTREL RVLYGPRLDE |
| | RDCPGNWTWP ENSQQTPMCQ |
| 421 | AWGNPLPELK CLKDGTFPLP IGESVTVTRD LEGTYLCRAR |
| | STQGEVTRKV TVNVLSPRYE |
| 481 | IVIITVVAAA VIMGTAGLST YLYNRQRKIK KYRLQQAQKG |
| | TPMKPNTQAT PP |

In accordance with the presently disclosed subject matter, an “ICAM-1 nucleic acid molecule” refers to a polynucleotide encoding an ICAM-1 polypeptide. An exemplary nucleic acid sequence encoding amino acids 481 to 507 of SEQ ID NO: 61 comprises or consists of the nucleotide sequence set forth in SEQ ID NO: 62, which is provided below.

[SEQ ID NO: 62]
ATTGTCATCATCACTGTGGTAGCAGCCGCAGTCATAATGGGCACTGCAGG
CCTCAGCACGTACCTCTATAACCGCCAGCGG

5.4.3.3. Hinge/Spacer Region

In certain embodiments, a CAR additionally comprises a hinge/spacer region that links the extracellular antigen-binding domain to the transmembrane domain. The hinge/spacer region can be flexible enough to allow the antigen binding domain to orient in different directions to facilitate antigen recognition. In certain embodiments, the hinge/spacer region of the CAR can comprise a native or modified hinge region of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 peptide, a NKG2D peptide, a synthetic polypeptide (not based on a protein associated with the immune response), or a combination thereof. The hinge/spacer region can be the hinge region from IgGl, or the CH₂CH₃ region of immunoglobulin and portions of CD3, a portion of a CD28 polypeptide (e.g., a portion of SEQ ID NO: 25), a portion of a CD8 polypeptide (e.g., a portion of SEQ ID NO: 45, or a portion of SEQ ID NO: 46), a variation of any of the foregoing which is at least about 80%, at least about 85%, at least about 90%, at least about 95%, or at least about 100% homologous or identical thereto, or a synthetic spacer sequence.

CD28

In certain embodiments, the hinge/spacer region of a presently disclosed CAR comprises a native or modified hinge region of a CD28 polypeptide as described herein. In certain embodiments, the hinge/spacer region of the CAR comprises a CD28 polypeptide comprising or consisting of amino acids 114 to 152 of SEQ ID NO: 25. An exemplary nucleic acid sequence encoding amino acids 114 to 152 of SEQ ID NO: 25 comprises or consists of the nucleotide sequence set forth in SEQ ID NO: 63, which is provided below.

[SEQ ID NO: 63]
IEVMYPPPYLDNEKSNGTIIHVKGKHLCPSPLFPGPSKP

CD84

In certain embodiments, the hinge/spacer region of a presently disclosed CAR comprises a native or modified hinge region of a CD84 polypeptide as described herein. In certain embodiments, the hinge/spacer region of the CAR comprises a CD84 polypeptide comprising or consisting of an amino acid sequence of amino acids 187 to 225 of SEQ ID NO: 50. An exemplary nucleic acid sequence encoding amino acids 187 to 225 of SEQ ID NO: 50 comprises or consists of the nucleotide sequence set forth in SEQ ID NO: 64, which is provided below.

[SEQ ID NO: 64]
CAAGAGCTGACTTACACGTGTACAGCCCAGAACCCTGTCAGCAACAATTC
TGACTCCATCTCTGCCCGGCAGCTCTGTGCAGACATCGCAATGGGCTTCC
GTACTCACCACACCGGG

CD166

In certain embodiments, the hinge/spacer region of a presently disclosed CAR comprises a native or modified hinge region of a CD166 polypeptide as described herein. In certain embodiments, the hinge/spacer region of the CAR comprises a CD166 polypeptide comprising or consisting of amino acids 489 to 527 of SEQ ID NO: 52. An exemplary nucleic acid sequence encoding amino acids 489 to 527 of SEQ ID NO: 52 comprises or consists of the nucleotide sequence set forth in SEQ ID NO: 65, which is provided below.

[SEQ ID NO: 65 ]
ACCAACTGGAGAGAACAGTAAACTCCTTGAATGTCTCTGCTATAAGTATT
CCAGAACACGATGAGGCAGACGAGATAAGTGATGAAAACAGAGAAAAGGT
GAATGACCAGGCAAAA

In certain embodiments, the CD166 polypeptide comprised in the hinge/spacer region of a presently disclosed CAR comprises or consists of amino acids 484 to 527 of SEQ ID NO: 52. In certain embodiments, the hinge/spacer region of the CAR comprises a CD166 comprising or consisting of amino acids 506 to 527 of SEQ ID NO: 52. In certain embodiments, the hinge/spacer region of the CAR comprises a CD166 polypeptide comprising or consisting of amino acids 517 to 527 of SEQ ID NO: 52. In certain embodiments, the hinge/spacer region of the CAR comprises a CD166 polypeptide comprising or consisting of the amino acid sequence set forth in SEQ ID NO: 66 or SEQ ID NO: 67.

[SEQ ID NO: 66]
NQLERTVNSLNVPAISIPEHDEADEISDENREKVNDQAK
[SEQ ID NO: 67]
AAANQLERTVNSLNVSAISIPEHDEADEISDENREKVNDQAK

In certain embodiments, the CD166 polypeptide comprised in the hinge/spacer region and the transmembrane domain of a presently disclosed CAR comprises or consists of the amino acid sequence set forth in SEQ ID NO: 68, SEQ ID NO: 69, SEQ ID NO: 70, SEQ ID NO: 71, SEQ ID NO: 72, SEQ ID NO: 73, or SEQ ID NO: 74.

[SEQ ID NO: 68]
PEHDEADEISDENREKVNDQAKLIVGIVVGLLLAALVAGVVYWLYMKK
[SEQ ID NO: 69]
ENREKVNDQAKLIVGIVVGLLLAALVAGVVYWLYMKK
[SEQ ID NO: 70]
NQLERTVNSLNVPAISIPEHDEADEISDENREKVNDQAKLIVGIVVGLLL
AALVAGVVYWLYMKK
[SEQ ID NO: 71]
TCTAENQLERTVNSLNVSAISIPEHDEADEISDENREKVNDQAKLIVGIV
VGLLLAALVAGVVYWL
[SEQ ID NO: 72]
PEHDEADEISDENREKVNDQAKLIVGIVVGLLLAALVAGVVYWL
[SEQ ID NO: 73]
NQLERTVNSLNVSAISIPEHDEADEISDENREKVNDQAKLIVGIVVGLLL
AALVAGVVYWL
[SEQ ID NO: 74]
AAANQLERTVNSLNVSAISIPEHDEADEISDENREKVNDQAKLIVGIVVG
LLLAALVAGVVYWLYMKK

CD8a

In certain embodiments, the hinge/spacer region of a presently disclosed CAR comprises a native or modified hinge region of a CD8a polypeptide as described herein. In certain embodiments, the hinge/spacer region of the CAR comprises a CD8a polypeptide comprising or consisting of amino acids 137 to 182 of SEQ ID NO: 54. An exemplary nucleic acid sequence encoding amino acids 137 to 182 of SEQ ID NO: 54 comprises or consists of the nucleotide sequence set forth in SEQ ID NO: 75, which is provided below.

[SEQ ID NO: 75]
cccaccacgacgccagcgccgcgaccaccaacaccggcgcccaccatcgc
gtcgcagcccctgtccctgcgcccagaggcgtgccggccagcggcggggg
gcgcagtgcacacgagggggctggacttcgcctgtgat

CD8b

In certain embodiments, the hinge/spacer region of a presently disclosed CAR comprises a native or modified hinge region of a CD8b polypeptide as described herein. In certain embodiments, the hinge/spacer region of the CAR comprises a CD8b polypeptide comprising or consisting of amino acids 132 to 170 of SEQ ID NO: 56. An exemplary nucleic acid sequence encoding the amino acids 132 to 170 of SEQ ID NO: 56 comprises or consists of the nucleotide sequence set forth in SEQ ID NO: 76, which is provided below.

[SEQ ID NO: 76]
CTGAGTGTGGTTGATTTCCTTCCCACCACTGCCCAGCCCACCAAGAAGTC
CACCCTCAAGAAGAGAGTGTGCCGGTTACCCAGGCCAGAGACCCAGAAGG
GCCCACTTTGTAGCCCC

ICOS

In certain embodiments, the hinge/spacer region of a presently disclosed CAR comprises a native or modified hinge region of an ICOS polypeptide as described herein. In certain embodiments, the hinge/spacer region of the CAR comprises an ICOS polypeptide comprising or consisting of amino acids 102 to 140 of SEQ ID NO: 27. An exemplary nucleic acid sequence encoding amino acids 102 to 140 of SEQ ID NO: 27 comprises or consists of the nucleotide sequence set forth in SEQ ID NO: 77, which is provided below.

[SEQ ID NO: 77]
tctcatgccaactattacttctgcaacctatcaatttttgatcctcctcc
ttttaaagtaactcttacaggaggatatttgcatatttatgaatcacaac
tttgttgccagctgaag

CTLA-4

In certain embodiments, the hinge/spacer region of a presently disclosed CAR comprises a native or modified hinge region of a CTLA-4 polypeptide as described herein. In certain embodiments, the hinge/spacer region of the CAR comprises a CTLA-4 polypeptide comprising or consisting of amino acids 123 to 161 of SEQ ID NO: 59. An exemplary nucleic acid sequence encoding amino acids 123 to 161 of SEQ ID NO: 59 comprises or consists of the nucleotide sequence set forth in SEQ ID NO: 78, which is provided below.

[SEQ ID NO: 78]
GACACGGGACTCTACATCTGCAAGGTGGAGCTCATGTACCCACCGCCATA
CTACCTGGGCATAGGCAACGGAACCCAGATTTATGTAATTGATCCAGAAC
CGTGCCCAGATTCTGAC

ICAM-1

In certain embodiments, the hinge/spacer region of a presently disclosed CAR comprises a native or modified hinge region of a ICAM-1 polypeptide as described herein. In certain embodiments, the hinge/spacer region of the CAR comprises an ICAM-1 polypeptide comprising or consisting of amino acids 442 to 480 of SEQ ID NO: 61. An exemplary nucleic acid sequence encoding amino acids 442 to 480 of SEQ ID NO: 61 comprises or consists of the nucleotide sequence set forth in SEQ ID NO: 79 as provided below.

[SEQ ID NO: 79]
GGGGAATCAGTGACTGTCACTCGAGATCTTGAGGGCACCTACCTCTGTCG
GGCCAGGAGCACTCAAGGGGAGGTCACCCGCAAGGTGACCGTGAATGTGC
TCTCCCCCCGGTATGAG

In certain embodiments, the hinge/spacer region is positioned between the extracellular antigen-binding domain and the transmembrane domain. In certain embodiments, the hinge/spacer region comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 peptide, a NKG2D peptide, a synthetic polypeptide (not based on a protein associated with the immune response), or a combination thereof. In certain embodiments, the transmembrane domain comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 peptide, a NKG2D peptide, a synthetic polypeptide (not based on a protein associated with the immune response), or a combination thereof.

In certain embodiments, the transmembrane domain and the hinge/spacer region are derived from the same molecule. In certain embodiments, the transmembrane domain and the hinge/spacer region are derived from different molecules. In certain embodiments, the hinge/spacer region of the CAR comprises a CD28 polypeptide and the transmembrane domain of the CAR comprises a CD28 polypeptide. In certain embodiments, the hinge/spacer region of the CAR comprises a CD28 polypeptide and the transmembrane domain of the CAR comprises a CD28 polypeptide. In certain embodiments, the hinge/spacer region of the CAR comprises a CD84 polypeptide and the transmembrane domain of the CAR comprises a CD84 polypeptide. In certain embodiments, the hinge/spacer region of the CAR comprises a CD166 polypeptide and the transmembrane domain of the CAR comprises a CD166 polypeptide. In certain embodiments, the hinge/spacer region of the CAR comprises a CD8a polypeptide and the transmembrane domain of the CAR comprises a CD8a polypeptide. In certain embodiments, the hinge/spacer region of the CAR comprises a CD8b polypeptide and the transmembrane domain of the CAR comprises a CD8b polypeptide. In certain embodiments, the hinge/spacer region of the CAR comprises a CD28 polypeptide and the transmembrane domain of the CAR comprises an ICOS polypeptide.

5.4.3.4. Intracellular Signaling Domain of a CAR

A. CD3ζ

In certain non-limiting embodiments, the CAR comprises an intracellular signaling domain. In certain non-limiting embodiments, the intracellular signaling domain of the CAR comprises a CD3ζ polypeptide, which can activate or stimulate a cell (e.g., a cell of the lymphoid lineage, e.g., a T cell). Wild type (“native”) CD3ζ comprises three immunoreceptor tyrosine-based activation motifs (“ITAMs”) (e.g., ITAM1, ITAM2 and ITAM3), three basic-rich stretch (BRS) regions (BRS1, BRS2 and BRS3), and transmits an activation signal to the cell (e.g., a cell of the lymphoid lineage, e.g., a T cell) after antigen is bound. The intracellular signaling domain of the native CD3ζ-chain is the primary transmitter of signals from endogenous TCRs. CD3ζ, as used in embodiments herein, is not native CD3ζ but is a modified CD3. In certain embodiments, the modified CD3ζ polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_932170 (SEQ ID No: 80), or a fragment thereof. In certain embodiments, the modified CD3ζ polypeptide comprises or consists of an amino acid sequence that is a consecutive portion of SEQ ID NO: 80, which is at least 20, or at least 30, or at least 40, or at least 50, or at least 100, or at least 110, or at least 113, and up to 164 amino acids in length. Alternatively or additionally, in certain embodiments, the modified CD3ζ polypeptide comprises or consists of an amino acid sequence of amino acids 1 to 50, 50 to 100, 100 to 150, 50 to 164, 52 to 164, 55 to 164, or 150 to 164 of SEQ ID NO: 80. In certain embodiments, the modified CD3ζ polypeptide comprises or consists of amino acids 52 to 164 of SEQ ID NO: 80.

SEQ ID NO: 80 is provided below:

[SEQ ID NO: 80]

| | |
|---|---|
| 1 | MKWKALFTAA ILQAQLPITE AQSFGLLDPK LCYLLDGILF |
| | IYGVILTALF LRVKFSRSAD |
| 61 | APAYQQGQNQ LYNELNLGRR EEYDVLDKRR GRDPEMGGKP |
| | QRRKNPQEGL YNELQKDKMA |
| 121 | EAYSEIGMKG ERRRGKGHDG LYQGLSTATK DTYDALHMQA |
| | LPPR |

In certain embodiments, the intracellular signaling domain of the CAR comprises a modified human CD3ζ polypeptide. In certain embodiments, the modified human CD3ζ polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence SEQ ID NO: 81 or a fragment thereof, and/or may optionally comprise up to one or up to two or up to three conservative amino acid substitutions. SEQ ID NO: 81 is provided below:

[SEQ ID NO: 81]
RVKFSRSADA PAYQQGQNQL YNELNLGRRE EYDVLDKRRG
RDPEMGGKPR RKNPQEGLYN ELQKDKMAEA YSEIGMKGER
RRGKGHDGLY QGLSTATKDT YDALHMQALP PR

An exemplary nucleic acid sequence encoding the amino acid sequence of SEQ ID NO: 81 comprises or consists of the nucleotide sequence set forth in SEQ ID NO: 82, which is provided below.

[SEQ ID NO: 82 ]
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGC

In certain embodiments, the intracellular signaling domain of the CAR comprises a modified CD3ζ polypeptide. In certain embodiments, the intracellular signaling domain of the CAR comprises a modified human CD3ζ polypeptide. In certain embodiments, the modified CD3ζ polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to SEQ ID NO: 83 or a fragment thereof, and/or may optionally comprise up to one or up to two or up to three conservative amino acid substitutions. SEQ ID NO: 83 is provided below:

[SEQ ID NO: 83]
RVKFSRSADA PAYQQGQNQL YNELNLGRRE EYDVLDKRRG
RDPEMGGKPR RKNPQEGLFN ELQKDKMAEA FSEIGMKGER
RRGKGHDGLF QGLSTATKDT FDALHMQALP PR

An exemplary nucleic acid sequence encoding the amino acid sequence of SEQ ID NO: 83 comprises or consists of the nucleotide sequence set forth in SEQ ID NO: 84, which is provided below.

[SEQ ID NO: 84]
agagtgaagttcagcaggagcgcagacgcccccgcgtaccagcagggcca
gaaccagctctataacgagctcaatctaggacgaagagaggagtacgatg
ttttggacaagagacgtggccgggaccctgagatggggggaaagccgaga
aggaagaaccctcaggaaggcctgtTcaatgaactgcagaaagataagat
ggcggaggcctTcagtgagattgggatgaaaggcgagcgccggaggggca
aggggcacgatggcctttTccaggggctcagtacagccaccaaggacacc
tTcgacgcccttcacatgcaggccctgccccctcgc

Immunoreceptor Tyrosine-Based Activation Motifs (ITAMs)

In certain embodiments, the intracellular signaling domain of the CAR comprises a modified CD3ζ polypeptide comprising or consisting of one, two or three ITAMs. In certain embodiments, the modified CD3ζ polypeptide comprises a native ITAM1 comprising or consisting of the amino acid sequence set forth in SEQ ID NO: 85.

[SEQ ID NO: 85]
QNQLYNELNLGRREEYDVLDKR

An exemplary nucleic acid sequence encoding the amino acid sequence of SEQ ID NO: 85 comprises or consists of the nucleotide sequence set forth in SEQ ID NO: 86, which is provided below.

[SEQ ID NO: 86]
cagaaccagctctataacgagctcaatctaggacgaagagaggagtacga
tgttttggacaagaga

In certain embodiments, the modified CD3ζ polypeptide comprises an ITAM1 variant comprising one or more loss-of-function mutations. In certain embodiments, the modified CD3ζ polypeptide consists of one ITAM1 variant comprising or consisting of two loss-of-function mutations. In certain embodiments, each of the one or more (e.g., two) loss of function mutations comprises or consists of a mutation of a tyrosine residue in ITAM1. In certain embodiments, the ITAM1 variant (e.g., the variant consisting of two loss-of-function mutations) comprises or consists of the amino acid sequence set forth in SEQ ID NO: 87, which is provided below.

[SEQ ID NO: 87]
QNQLFNELNLGRREEFDVLDKR

An exemplary nucleic acid sequence encoding the amino acid sequence of SEQ ID NO: 87 comprises or consists of the nucleotide sequence set forth in SEQ ID NO: 88, which is provided below.

[SEQ ID NO: 88]
cagaaccagctctTtaacgagctcaatctaggacgaagagaggagtTcga
tgttttggacaagaga

In certain embodiments, the modified CD3ζ polypeptide comprises a native ITAM2 comprising or consisting of the amino acid sequence set forth in SEQ ID NO: 89, which is provided below.

[SEQ ID NO: 89]
QEGLYNELQKDKMAEAYSEIGMK

An exemplary nucleic acid sequence encoding the amino acid sequence of SEQ ID NO: 89 comprises or consists of the nucleotide sequence set forth in SEQ ID NO: 90, which is provided below.

[SEQ ID NO: 90]
caggaaggcctgtacaatgaactgcagaaagataagatggcggaggccta
cagtgagattgggatgaaa

In certain embodiments, the modified CD3ζ polypeptide comprises an ITAM2 variant comprising one or more loss-of-function mutations. In certain embodiments, the modified CD3ζ polypeptide consists of one ITAM2 variant comprising or consisting of two loss-of-function mutations. In certain embodiments, each of the one or more (e.g., two) the loss of function mutations comprises a mutation of a tyrosine residue in ITAM2. In certain embodiments, the ITAM2 variant (e.g., a variant consisting of two loss-of-function mutations) comprises or consists of the amino acid sequence set forth in SEQ ID NO: 91, which is provided below.

[SEQ ID NO: 91]
QEGLFNELQKDKMAEAFSEIGMK

An exemplary nucleic acid sequence encoding the amino acid sequence of SEQ ID NO: 91 comprises or consists of the nucleotide sequence set forth in SEQ ID NO: 92, which is provided below.

[SEQ ID NO: 92]
caggaaggcctgtTcaatgaactgcagaaagataagatggcggaggcctT
cagtgagattgggatgaaa

In certain embodiments, the modified CD3ζ polypeptide comprises a native ITAM3 comprising the amino acid sequence set forth in SEQ ID NO: 93, which is provided below.

[SEQ ID NO: 93]
HDGLYQGLSTATKDTYDALHMQ

An exemplary nucleic acid sequence encoding the amino acid sequence of SEQ ID NO: 93 comprises or consists of the nucleotide sequence set forth in SEQ ID NO: 94, which is provided below.

[SEQ ID NO: 94]
cacgatggcctttaccagggtctcagtacagccaccaaggacacctacga
cgcccttcacatgcag

In certain embodiments, the modified CD3ζ polypeptide comprises an ITAM3 variant comprising one or more loss-of-function mutations. In certain embodiments, the modified CD3ζ polypeptide consists of one ITAM3 variant comprising or consisting of two loss-of-function mutations. In certain embodiments, each of the one or more (e.g., two) the loss of function mutations comprises a mutation of a tyrosine residue in ITAM3. In certain embodiments, the ITAM3 variant (e.g., a variant consisting of two loss-of-function mutations) comprises or consists of the amino acid sequence set forth in SEQ ID NO: 95, which is provided below.

[SEQ ID NO: 95]
HDGLFQGLSTATKDTFDALHMQ

An exemplary nucleic acid sequence encoding the amino acid sequence of SEQ ID NO: 95 comprises or consists of the nucleotide sequence set forth in SEQ ID NO: 96, which is provided below.

[SEQ ID NO: 96]
cacgatggcctttTccaggggctcagtacagccaccaaggacacctTcga
cgcccttcacatgcag

In certain embodiments, the intracellular signaling domain of the CAR comprises a modified CD3ζ polypeptide comprising or consisting essentially of or consisting of an ITAM1 variant comprising one or more loss-of-function mutations, an ITAM2 variant comprising one or more loss-of-function mutations, an ITAM3 variant comprising one or more loss-of-function mutations, or a combination thereof. In certain embodiments, the intracellular signaling domain of the CAR comprises a modified CD3ζ polypeptide comprising an ITAM2 variant comprising one or more (e.g., two) loss-of-function mutations and an ITAM3 variant comprising one or more (e.g., two) loss-of-function mutations. In certain embodiments, the intracellular signaling domain of the CAR comprises a modified CD3ζ polypeptide comprising a native ITAM1, an ITAM2 variant comprising or consisting of two loss-of-function mutations and an ITAM3 variant comprising or consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 85, an ITAM2 variant consisting of the amino acid sequence set forth in SEQ ID NO: 91, and an ITAM3 variant consisting of the amino acid sequence set forth in SEQ ID NO: 95 (e.g., a construct designated as “1XX”). In certain embodiments, the modified CD3ζ polypeptide comprising or consists of the amino acid sequence set forth in SEQ ID NO: 83.

In certain embodiments, the intracellular signaling domain of the CAR comprises a modified CD3ζ polypeptide comprising an ITAM1 variant comprising one or more (e.g., two) loss-of-function mutations and an ITAM3 variant comprising one or more (e.g., two) loss-of-function mutations. In certain embodiments, the intracellular signaling domain of the CAR comprises a modified CD3 polypeptide comprising an ITAM1 variant comprising two loss-of-function mutations, a native ITAM2, and an ITAM3 variant comprising two loss-of-function mutations. In certain embodiments, the intracellular signaling domain of the CAR comprises a modified CD3ζ polypeptide comprising an ITAM1 variant consisting of the amino acid sequence set forth in SEQ ID NO: 87, a native ITAM2 consisting of the amino acid sequence set forth in SEQ ID NO: 89, and an ITAM3 variant consisting of the amino acid sequence set forth in SEQ ID NO: 95 (e.g., a construct designated as “X2X”).

In certain embodiments, the intracellular signaling domain of the CAR comprises a modified CD3ζ polypeptide comprising an ITAM1 variant comprising one or more (e.g., two) loss-of-function mutations and an ITAM2 variant comprising one or more (e.g., two) loss-of-function mutations. In certain embodiments, the intracellular signaling domain of the CAR comprises a modified CD3 polypeptide comprising an ITAM1 variant comprising or consisting of two loss-of-function mutations, an ITAM2 variant comprising or consisting of wo loss-of-function mutations, and a native ITAM3. In certain embodiments, the intracellular signaling domain of the CAR comprises a modified CD3ζ polypeptide comprising an ITAM1 variant consisting of the amino acid sequence set forth in SEQ ID NO: 87, an ITAM2 variant consisting of the amino acid sequence set forth in SEQ ID NO: 91, and a native ITAM3 consisting of the amino acid sequence set forth in SEQ ID NO: 93 (e.g., a construct designated as “XX3”).

In certain embodiments, the intracellular signaling domain of the CAR comprises a modified CD3ζ polypeptide comprising an ITAM1 variant comprising one or more (e.g., two) loss-of-function mutations. In certain embodiments, the intracellular signaling domain of the CAR comprises a modified CD3ζ polypeptide comprising an ITAM1 variant comprising or consisting of two loss-of-function mutations, a native ITAM2, and a native ITAM3. In certain embodiments, the intracellular signaling domain of the CAR comprises a modified CD3ζ polypeptide comprising an ITAM1 variant consisting of the amino acid sequence set forth in SEQ ID NO: 87, a native ITAM2 consisting of the amino acid sequence set forth in SEQ ID NO: 89, and a native ITAM3 consisting of the amino acid sequence set forth in SEQ ID NO: 93 (e.g., a construct designated as “X23”).

In certain embodiments, the intracellular signaling domain of the CAR comprises a modified CD3ζ polypeptide comprising a native ITAM1, a native ITAM2, and an ITAM3 variant comprising one or more (e.g., two) loss-of-function mutations. In certain embodiments, the intracellular signaling domain of the CAR comprises a modified CD3ζ polypeptide comprising a native ITAM1, a native ITAM2, and an ITAM1 variant comprising or consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 85, a native ITAM2 consisting of the amino acid sequence set forth in SEQ ID NO: 89, and an ITAM3 variant consisting of the amino acid sequence set forth in SEQ ID NO: 95 (e.g., a construct designated as “12X”).

In certain embodiments, the intracellular signaling domain of the CAR comprises a modified CD3ζ polypeptide comprising a native ITAM1, an ITAM2 variant comprising one or more (e.g., two) loss-of-function mutations, and a native ITAM3. In certain embodiments, the intracellular signaling domain of the CAR comprises a modified CD3ζ polypeptide comprising a native ITAM1, an ITAM2 variant comprising or consisting of two loss-of-function mutations, and a native ITAM3. In certain embodiments, the intracellular signaling domain of the CAR comprises a modified CD3ζ polypeptide comprising a native ITAM1 consisting of the amino acid sequence set forth in SEQ ID NO: 85, an ITAM2 variant consisting of the amino acid sequence set forth in SEQ ID NO: 91, and a native ITAM3 variant consisting of the amino acid sequence set forth in SEQ ID NO: 93 (e.g., a construct designated as “1X3”).

In certain embodiments, the intracellular signaling domain of the CAR comprises a modified CD3ζ polypeptide comprising a deletion of one or two ITAMs. In certain embodiments, the modified CD3ζ polypeptide comprises or consists of a deletion of ITAM1 and ITAM2, e.g., the modified CD3ζ polypeptide comprises a native ITAM3 or an ITAM3 variant, and does not comprise an ITAM1 or an ITAM2. In certain embodiments, the modified CD3ζ polypeptide comprises a native ITAM3 consisting of the amino acid sequence set forth in SEQ ID NO: 93, and does not comprise an ITAM1 (native or modified), or an ITAM2 (native or modified) (e.g., a construct designated as “D12”).

In certain embodiments, the modified CD3ζ polypeptide comprises or consists of a deletion of ITAM2 and ITAM3, e.g., the modified CD3ζ polypeptide comprises a native ITAM1 or an ITAM1 variant, and does not comprise an ITAM2 or an ITAM3. In certain embodiments, the modified CD3ζ polypeptide comprises a native ITAM1 consisting of the amino acid sequence set forth in SEQ ID NO: 85, and does not comprise an ITAM2 (native or modified), or an ITAM3 (native or modified) (e.g., a construct designated as “D23”).

In certain embodiments, the modified CD3ζ polypeptide comprises or consists of a deletion of ITAM1 and ITAM3, e.g., the modified CD3ζ polypeptide comprises a native ITAM2 or an ITAM2 variant, and does not comprise an ITAM1 or an ITAM3. In certain embodiments, the modified CD3ζ polypeptide comprises a native ITAM2 consisting of the amino acid sequence set forth in SEQ ID NO: 89, and does not comprise an ITAM1 (native or modified), or an ITAM3 (native or modified) (e.g., a construct designated as “D13”).

In certain embodiments, the modified CD3ζ polypeptide comprises or consists of a deletion of ITAM1, e.g., the modified CD3ζ polypeptide comprises a native ITAM2 or an ITAM2 variant, and a native ITAM3 or an ITAM3 variant, and does not comprise an ITAM1 (native or modified).

In certain embodiments, the modified CD3ζ polypeptide comprises or consists of a deletion of ITAM2, e.g., the modified CD3ζ polypeptide comprises a native ITAM1 or an ITAM1 variant, and a native ITAM3 or an ITAM3 variant, and does not comprise an ITAM2 (native or modified).

In certain embodiments, the modified CD3ζ polypeptide comprises or consists of a deletion of ITAM3, e.g., the modified CD3ζ polypeptide comprises a native ITAM1 or an ITAM1 variant, and a native ITAM2 or an ITAM2 variant, and does not comprise an ITAM3 (native or modified).

B. Co-Stimulatory Signaling Region

In certain embodiments, the intracellular signaling domain of the CAR further comprises at least a co-stimulatory signaling region. In certain embodiments, the at least one co-stimulatory signaling region comprises an intracellular domain of a co-stimulatory molecule (e.g., a co-stimulatory molecule disclosed in Section 5.2.2). In certain embodiments, the co-stimulatory molecule is selected from the group consisting of CD28, 4-1BB, OX40, CD27, CD40, ICOS, DAP-10, CD2, and NKG2D.

The at least one co-stimulatory signaling region can include a CD28 polypeptide, a 4-1BB polypeptide, an OX40 polypeptide, an ICOS polypeptide, a DAP-10 polypeptide, a CD27 polypeptide, a CD40 polypeptide, a CD2 polypeptide, a NKG2D polypeptide or a combination thereof. The at least one co-stimulatory signaling region can include an intracellular domain of CD28 or a portion thereof, an intracellular domain of 4-1BB or a portion thereof, an intracellular domain of OX40 or a portion thereof, an intracellular domain of ICOS or a portion thereof, an intracellular domain of DAP-10 or a portion thereof, an intracellular domain of CD27 or a portion thereof, an intracellular domain of CD40 or a portion thereof, an intracellular domain of CD2 or a portion thereof, an intracellular domain of NKG2D or a portion thereof, or a combination thereof.

The co-stimulatory molecule can bind to a co-stimulatory ligand (e.g., co-stimulatory ligand disclosed in Section 5.2.1). As one example, a 4-1BB ligand (i.e., 4-1BBL) may bind to 4-1BB (also known as “CD137”) for providing an intracellular signal that in combination with a CAR signal induces an effector cell function of the CAR′ T cell. CARs comprising an intracellular signaling domain that comprises a co-stimulatory signaling region comprising a 4-1BB polypeptide, an ICOS polypeptide, or a DAP-10 polypeptide are disclosed in U.S. Pat. No. 7,446,190, which is herein incorporated by reference in its entirety.

In certain embodiments, the intracellular signaling domain of the CAR comprises a co-stimulatory signaling region that comprises a CD28 polypeptide (e.g., an intracellular domain of CD28). In certain embodiments, the intracellular signaling domain of the CAR comprises a co-stimulatory signaling region that comprises an intracellular domain of human CD28 or a portion thereof. In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 25, or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 25, which is at least 20, or at least 30, or at least 40, or at least 50, and up to 220 amino acids in length. Alternatively or additionally, in non-limiting various embodiments, the CD28 polypeptide comprises or consists of an amino acid sequence of amino acids 1 to 220, 1 to 50, 50 to 100, 100 to 150, 150 to 200, 180 to 220, 180 to 219, or 200 to 220 of SEQ ID NO: 25. In certain embodiments, the intracellular signaling domain of the CAR comprises a co-stimulatory signaling region that comprises an intracellular domain of CD28 or a portion thereof. In certain embodiments, the intracellular signaling domain of the CAR comprises a co-stimulatory signaling region that comprises an intracellular domain of human CD28 or a portion thereof. In certain embodiments, the intracellular signaling domain of the CAR comprises a co-stimulatory signaling region that comprises a CD28 polypeptide comprising or consisting of amino acids 180 to 220 of SEQ ID NO: 25. In certain embodiments, the intracellular signaling domain of the CAR comprises a co-stimulatory signaling region that comprises a CD28 polypeptide comprising or consisting of amino acids 180 to 219 of SEQ ID NO: 25.

In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to SEQ ID NO: 101 or a fragment thereof, and/or may optionally comprise up to one or up to two or up to three conservative amino acid substitutions. In certain embodiments, the CD28 polypeptide comprises or consists of the amino acid sequence set forth in SEQ ID NO: 101 (or amino acids 180 to 220 of SEQ ID NO: 25). SEQ ID NO: 101 is provided below:

[SEQ ID NO: 101]
RSKRSRLLHS DYMNMTPRRP GPTRKHYQPY APPRDFAAYR S.

An exemplary nucleic acid sequence encoding the amino acid sequence of SEQ ID NO: 101 comprises of consists of the nucleotide sequence set forth in SEQ ID NO: 102, which is provided below.

[SEQ ID NO: 102]
AGGAGTAAGAGGAGCAGGCTCCTGCACAGTGACTACATGAACATGACTCCC
CGCCGCCCCGGGCCCACCCGCAAGCATTACCAGCCCTATGCCCCACCACGC
GACTTCGCAGCCTATCGCTCC

In certain embodiments, the intracellular signaling domain of the CAR comprises a de-immunized intracellular domain of human CD28 or a portion thereof. In certain embodiments, the de-immunized intracellular domain of human CD28 or the portion thereof comprises or consists of an amino acid sequence that is at least about 80%, at least about 85%, at least about 90%, at least about 95%, at least about 96%, at least about 97%, at least about 98%, or at least about 99%, at least about 100% homologous or identical to SEQ ID NO: 103 or a fragment thereof, and/or may optionally comprise up to one or up to two or up to three conservative amino acid substitutions. In certain embodiments, the CD28 polypeptide comprises or consists of the amino acid sequence set forth in SEQ ID NO: 103. SEQ ID NO: 103 is provided below:

[SEQ ID NO: 103]
RSKRSRLLHS DYMNMTPRRP GPTRKHYQPY APPRDFAAYR K

In certain embodiments, the intracellular signaling domain of the CAR comprises a co-stimulatory signaling region that comprises an intracellular domain of murine CD28 or a portion thereof. In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_031668.3 (SEQ ID NO: 97), or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 97, which is at least about 20, or at least about 30, or at least about 40, or at least about 50, and up to 218 amino acids in length. Alternatively or additionally, in non-limiting various embodiments, the CD28 polypeptide comprises or consists of an amino acid sequence of amino acids 1 to 218, 1 to 50, 50 to 100, 100 to 150, 114 to 220, 150 to 200, 178 to 218, or 200 to 218 of SEQ ID NO: 97. In certain embodiments, the co-stimulatory signaling region of a presently disclosed CAR comprises a CD28 polypeptide that comprises or consists of amino acids 178 to 218 of SEQ ID NO: 97.

SEQ ID NO: 97 is provided below:

[SEQ ID NO: 97]

| | |
|---|---|
| 1 | MTLRLLFLAL NFFSVQVTEN KILVKQSPLL VVDSNEVSLS |
| | CRYSYNLLAK EFRASLYKGV |
| 61 | NSDVEVCVGN GNFTYQPQFR SNAEFNCDGD FDNETVTFRL |
| | WNLHVNHTDI YFCKIEFMYP |
| 121 | PPYLDNERSN GTIIHIKEKH LCHTQSSPKL FWALVVVAGV |
| | LFCYGLLVTV ALCVIWTNSR |
| 181 | RNRLLQSDYM NMTPRRPGLT RKPYQPYAPA RDFAAYRP |

An exemplary nucleic acid sequence encoding amino acids 178 to 218 of SEQ ID NO: 97 comprises of consists of the nucleotide sequence set forth in SEQ ID NO: 98, which is provided below.

[SEQ ID NO: 98]
aat agtagaagga acagactcct tcaaagtgac tacatgaaca
tgactccccg gaggcctggg ctcactcgaa agccttacca
gccctacgcc cctgccagag actttgcagc gtaccgcccc

In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence of SEQ ID NO: 99 or fragments thereof, and/or may optionally comprise up to one or up to two or up to three conservative amino acid substitutions. In certain embodiments, the CD28 polypeptide comprises or consists of the amino acid sequence set forth in SEQ ID NO: 99. SEQ ID NO: 99 is provided below:

[SEQ ID NO: 99]
NSRRNRLLQS DYMNMTPRRP GLTRKPYQPY APARDFAAYR P.

An exemplary nucleic acid sequence encoding the amino acid sequence of SEQ ID NO: 99 comprises of consists of the nucleotide sequence set forth in SEQ ID NO: 100, which is provided below.

[SEQ ID NO: 100]
AATAGTAGAAGGAACAGACTCCTTCAAAGTGACTACATGAACATGACTCCC
CGGAGGCCTGGGCTCACTCGAAAGCCTTACCAGCCCTACGCCCCTGCCAGA
GACTTTGCAGCGTACCGCCCC

In certain embodiments, the intracellular signaling domain of the CAR comprises a co-stimulatory signaling region that comprises two co-stimulatory molecules, e.g., co-stimulatory signaling regions of CD28 and 4-1BB or co-stimulatory signaling regions of CD28 and OX40.

In certain embodiments, the intracellular signaling domain of the CAR comprises a co-stimulatory signaling region that comprises a 4-1BB polypeptide. In certain embodiments, the 4-1BB polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 24 or a fragment thereof, and/or may optionally comprise up to one or up to two or up to three conservative amino acid substitutions.

In certain embodiments, the intracellular signaling domain of the CAR comprises a co-stimulatory signaling region that comprises an intracellular domain of 4-1BB or a portion thereof. In certain embodiments, the intracellular signaling domain of the CAR comprises a co-stimulatory signaling region that comprises an intracellular domain of human 4-1BB or a portion thereof. In certain embodiments, the intracellular signaling domain of 4-1BB or portion thereof comprises or consists of an amino acid sequence that is at least about 80%, at least about 85%, at least about 90%, at least about 95%, at least about 96%, at least about 97%, at least about 98%, or at least about 99%, at least about 100% homologous or identical to SEQ ID NO: 3 or fragments thereof, and/or may optionally comprise up to one or up to two or up to three conservative amino acid substitutions. In certain embodiments, the intracellular domain of 4-1BB comprises or consists of an amino acid sequence set forth in SEQ ID NO: 3.

An exemplary nucleic acid sequence encoding the amino acid sequence of SEQ ID NO: 3 comprises of consists of the nucleotide sequence set forth in SEQ ID NO: 104, which is provided below.

[SEQ ID NO: 104]
AAACGGGGCAGAAAGAAACTCCTGTATATATTCAAACAACCATTTATGAGA
CCAGTACAAACTACTCAAGAGGAAGATGGCTGTAGCTGCCGATTTCCAGAA
GAAGAAGAAGGAGGATGTGAACTG

An OX40 polypeptide can comprise or have an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 26 or a fragments thereof, and/or may optionally comprise up to one or up to two or up to three conservative amino acid substitutions.

An ICOS polypeptide can comprise or have an amino acid sequence that is at least about 80%, at least about 85%, at least about 90%, at least about 95%, at least about 96%, at least about 97%, at least about 98%, or at least about 99%, at least about 100% homologous or identical homologous to the amino acid sequence set forth in SEQ ID NO: 27 or a fragment thereof, and/or may optionally comprise up to one or up to two or up to three conservative amino acid substitutions.

In certain embodiments, a presently disclosed CAR further comprises an inducible promoter, for expressing nucleic acid sequences in human cells. Promoters for use in expressing CAR genes can be a constitutive promoter, such as ubiquitin C (UbiC) promoter.

In certain embodiments, mutation sites and/or junction between domains/motifs/regions of the CAR derived from different proteins are de-immunized. Immunogenicity of junctions between different CAR moieties can be predicted using NetMHC 4.0 Server. For each peptide containing at least one amino acid from next moiety, binding affinity to HLA A, B and C, for all alleles, can be predicted. A score of immunogenicity of each peptide can be assigned for each peptide. Immunogenicity score can be calculated using the formula Immunogenicity score=[(50-binding affinity)*HLA frequency]ₙ. n is the number of prediction for each peptide.

5.4.3.5. Exemplary CARs

1928z CAR

In certain embodiments, the cell comprises a presently disclosed fusion polypeptide and a CAR comprising an extracellular antigen-binding domain that binds to CD19 (e.g., human CD19), a transmembrane domain comprising a CD28 polypeptide (e.g., human CD28 polypeptide, e.g., a transmembrane domain of CD28 (e.g., human CD28) or a portion thereof), an intracellular signaling domain comprising a CD3ζ polypeptide (e.g., a human CD3ζ polypeptide), and a co-stimulatory signaling domain comprising a CD28 polypeptide (e.g., human CD28 polypeptide, e.g., an intracellular domain of CD28 (e.g., human CD28) of a portion thereof). In certain embodiments, the CAR is designated as “1928z”. In certain embodiments, the CAR (e.g., 1928z) comprises an amino acid sequence that is at least about 85%, about 90%, about 95%, about 96%, about 97%, about 98%, about 99% or about 100% homologous or identical to the amino acid sequence set forth in SEQ ID NO: 105, which is provided below. In certain embodiments, the CAR (e.g., 1928z) comprises the amino acid sequence set forth in SEQ ID NO: 105. The CAR comprising the amino acid sequence set forth in SEQ ID NO: 105 is able to bind to CD19 (e.g., human CD19).

[SEQ ID NO: 105]
EVKLQQSGAELVRPGSSVKISCKASGYAFSSYWMNWVKQRPGQGLEWIGQI
YPGDGDTNYNGKFKGQATLTADKSSSTAYMQLSGLTSEDSAVYFCARKTIS
SVVDFYFDYWGQGTTVTVSSGGGGSGGGGSGGGGSDIELTQSPKFMSTSVG
DRVSVTCKASQNVGTNVAWYQQKPGQSPKPLIYSATYRNSGVPDRFTGSGS
GTDFTLTITNVQSKDLADYFCQQYNRYPYTSGGGTKLEIKRAAAIEVMYPP
PYLDNEKSNGTIIHVKGKHLCPSPLFPGPSKPFWVLVVVGGVLACYSLLVT
VAFIIFWVRSKRSRLLHSDYMNMTPRRPGPTRKHYQPYAPPRDFAAYRSRV
KFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDAL
HMQALPPR

An exemplary nucleic acid sequence encoding the amino acid sequence of SEQ ID NO: 105 comprises or consists of the nucleotide sequence set forth in SEQ ID NO: 106, which is provided below.

[SEQ ID NO: 106]
GAGGTGAAGCTGCAGCAGTCTGGGGCTGAGCTGGTGAGGCCTGGGTCCTCA
GTGAAGATTTCCTGCAAGGCTTCTGGCTATGCATTCAGTAGCTACTGGATG
AACTGGGTGAAGCAGAGGCCTGGACAGGGTCTTGAGTGGATTGGACAGATT
TATCCTGGAGATGGTGATACTAACTACAATGGAAAGTTCAAGGGTCAAGCC
ACACTGACTGCAGACAAATCCTCCAGCACAGCCTACATGCAGCTCAGCGGC
CTAACATCTGAGGACTCTGCGGTCTATTTCTGTGCAAGAAAGACCATTAGT
TCGGTAGTAGATTTCTACTTTGACTACTGGGGCCAAGGGACCACGGTCACC
GTCTCCTCAGGTGGAGGTGGATCAGGTGGAGGTGGATCTGGTGGAGGTGGA
TCTGACATTGAGCTCACCCAGTCTCCAAAATTCATGTCCACATCAGTAGGA
GACAGGGTCAGCGTCACCTGCAAGGCCAGTCAGAATGTGGGTACTAATGTA
GCCTGGTATCAACAGAAACCAGGACAATCTCCTAAACCACTGATTTACTCG
GCAACCTACCGGAACAGTGGAGTCCCTGATCGCTTCACAGGCAGTGGATCT
GGGACAGATTTCACTCTCACCATCACTAACGTGCAGTCTAAAGACTTGGCA
GACTATTTCTGTCAACAATATAACAGGTATCCGTACACGTCCGGAGGGGGG
ACCAAGCTGGAGATCAAACGGGCGGCCGCAATTGAAGTTATGTATCCTCCT
CCTTACCTAGACAATGAGAAGAGCAATGGAACCATTATCCATGTGAAAGGG
AAACACCTTTGTCCAAGTCCCCTATTTCCCGGACCTTCTAAGCCCTTTTGG
GTGCTGGTGGTGGTTGGTGGAGTCCTGGCTTGCTATAGCTTGCTAGTAACA
GTGGCCTTTATTATTTTCTGGGTGAGGAGTAAGAGGAGCAGGCTCCTGCAC
AGTGACTACATGAACATGACTCCCCGCCGCCCCGGGCCCACCCGCAAGCAT
TACCAGCCCTATGCCCCACCACGCGACTTCGCAGCCTATCGCTCCAGAGTG
AAGTTCAGCAGGAGCGCAGACGCCCCCGCGTACCAGCAGGGCCAGAACCAG
CTCTATAACGAGCTCAATCTAGGACGAAGAGAGGAGTACGATGTTTTGGAC
AAGAGACGTGGCCGGGACCCTGAGATGGGGGGAAAGCCGAGAAGGAAGAAC
CCTCAGGAAGGCCTGTACAATGAACTGCAGAAAGATAAGATGGCGGAGGCC
TACAGTGAGATTGGGATGAAAGGCGAGCGCCGGAGGGGCAAGGGGCACGAT
GGCCTTTACCAGGGTCTCAGTACAGCCACCAAGGACACCTACGACGCCCTT
CACATGCAGGCCCTGCCCCCTCGC

In certain embodiments, the CAR further comprises a CD8 leader. In certain embodiments, the CD8 leader comprises or has the amino acid sequence set forth in SEQ ID NO: 13.

An exemplary nucleic acid sequence encoding the amino acid sequence of SEQ ID NO: 13 is set forth in SEQ ID NO: 107, which is provided below.

[SEQ ID NO: 107]
ATGGCTCTCCCAGTGACTGCCCTACTGCTTCCCCTAGCGCTTCTCCTGCAT
GCA

1928z-1xx CAR

In certain embodiments, a presently disclosed CAR comprises an extracellular antigen-binding domain that binds to a CD19 polypeptide (e.g., a human CD19 polypeptide), a transmembrane domain comprising a CD28 polypeptide (e.g., human CD28 polypeptide, e.g., a transmembrane domain of CD28 (e.g., human CD28) or a portion thereof), a hinge/spacer region derived from a CD28 polypeptide (e.g., human CD28), an intracellular signaling domain comprising a modified CD3ζ polypeptide (e.g., a modified human CD3ζ polypeptide) comprising a native ITAM1, an ITAM2 variant consisting of two loss-of-function mutations, and an ITAM3 variant consisting of two loss-of-function mutations, and a co-stimulatory signaling region comprising a CD28 polypeptide (e.g., a human CD28 polypeptide, e.g., an intracellular domain of CD28 (e.g., human CD28) of a portion thereof). In certain embodiments, the CAR is designated as “1928z-1xx”. In certain embodiments, the CAR (e.g., 1928z-1xx) comprises an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 109, which is provided below. In certain embodiments, the CAR (e.g., 1928z-1xx) comprises the amino acid sequence set forth in SEQ ID NO: 109. The CAR comprising the amino acid sequence set forth in SEQ ID NO: 109 is able to bind to CD19 (e.g., human CD19).

[SEQ ID NO: 109]
EVKLQQSGAELVRPGSSVKISCKASGYAFSSYWMNWVKQRPGQGLEWIGQI
YPGDGDTNYNGKFKGQATLTADKSSSTAYMQLSGLTSEDSAVYFCARKTIS
SVVDFYFDYWGQGTTVTVSSGGGGSGGGGSGGGGSDIELTQSPKFMSTSVG
DRVSVTCKASQNVGTNVAWYQQKPGQSPKPLIYSATYRNSGVPDRFTGSGS
GTDFTLTITNVQSKDLADYFCQQYNRYPYTSGGGTKLEIKRAAAIEVMYPP
PYLDNEKSNGTIIHVKGKHLCPSPLFPGPSKPFWVLVVVGGVLACYSLLVT
VAFIIFWVRSKRSRLLHSDYMNMTPRRPGPTRKHYQPYAPPRDFAAYRSRV
KFSRSADAPAYQQGQNQLYNELNLGRREEYDVLDKRRGRDPEMGGKPRRKN
PQEGLFNELQKDKMAEAFSEIGMKGERRRGKGHDGLFQGLSTATKDTFDAL
HMQALPPR

An exemplary nucleic acid sequence encoding the amino acid sequence of SEQ ID NO: 109 comprises or consists of the nucleotide sequence set forth in SEQ ID NO: 110, which is provided below.

[SEQ ID NO: 110]
gaggtgaagctgcagcagtctggggctgagctggtgaggcctgggtcctca
gtgaagatttcctgcaaggcttctggctatgcattcagtagctactggatg
aactgggtgaagcagaggcctggacagggtcttgagtggattggacagatt
tatcctggagatggtgatactaactacaatggaaagttcaagggtcaagcc
acactgactgcagacaaatcctccagcacagcctacatgcagctcagcggc
ctaacatctgaggactctgcggtctatttctgtgcaagaaagaccattagt
tcggtagtagatttctactttgactactggggccaagggaccacggtcacc
gtctcctcaggtggaggtggatcaggtggaggtggatctggtggaggtgga
tctgacattgagctcacccagtctccaaaattcatgtccacatcagtagga
gacagggtcagcgtcacctgcaaggccagtcagaatgtgggtactaatgta
gcctggtatcaacagaaaccaggacaatctcctaaaccactgatttactcg
gcaacctaccggaacagtggagtccctgatcgcttcacaggcagtggatct
gggacagatttcactctcaccatcactaacgtgcagtctaaagacttggca
gactatttctgtcaacaatataacaggtatccgtacacgtccggagggggg
accaagctggagatcaaacgggcggccgcaattgaagttatgtatcctcct
ccttacctagacaatgagaagagcaatggaaccattatccatgtgaaaggg
aaacacctttgtccaagtcccctatttcccggaccttctaagcccttttgg
gtgctggtggtggttggtggagtcctggcttgctatagcttgctagtaaca
gtggcctttattattttctgggtgaggagtaagaggagcaggctcctgcac
agtgactacatgaacatgactccccgccgccccgggcccacccgcaagcat
taccagccctatgccccaccacgcgacttcgcagcctatcgctccagagtg
aagttcagcaggagcgcagacgcccccgcgtaccagcagggccagaaccag
ctctataacgagctcaatctaggacgaagagaggagtacgatgttttggac
aagagacgtggccgggaccctgagatggggggaaagccgagaaggaagaac
cctcaggaaggcctgtTcaatgaactgcagaaagataagatggcggaggcc
tTcagtgagattgggatgaaaggcgagcgccggaggggcaaggggcacgat
ggcctttTccaggggctcagtacagccaccaaggacacctTcgacgccctt
cacatgcaggccctgccccctcgctaa

In certain embodiments, the CAR further comprises a CD8 leader. In certain embodiments, the CD8 leader comprises or has the amino acid sequence set forth in SEQ ID NO: 13.

An exemplary nucleic acid sequence encoding the amino acid sequence of SEQ ID NO: 13 comprises or consists of the nucleotide sequence set forth in SEQ ID NO: 107.

5.4.4. TCR like Fusion Molecules

In certain embodiments, the antigen-recognizing receptor is a TCR like fusion molecule. Non-limiting examples of TCR fusion molecules include HLA-Independent TCR-based Chimeric Antigen Receptor (also known as “HIT-CAR”, e.g., those disclosed in International Patent Application No. PCT/US19/017525, which is incorporated by reference in its entirety), and T cell receptor fusion constructs (TRuCs) (e.g., those disclosed in Baeuerle et al., “Synthetic TRuC receptors engaging the complete T cell receptor for potent anti-tumor response,” Nature Communications volume 10, Article number: 2087 (2019), which is incorporated by reference in its entirety).

In certain embodiments, the TCR like fusion molecule comprises an antigen binding chain that comprises an extracellular antigen-binding domain and a constant domain, wherein the TCR like fusion molecule binds to an antigen in an HLA-independent manner. In certain embodiments, the constant domain comprises a T cell receptor constant region selected from the group consisting of a native or modified TRAC peptide, a native or modified TRBC peptide, a native or modified TRDC peptide, a native or modified TRGC peptide and any variants or functional fragments thereof. In certain embodiments, the constant domain comprises a native or modified TRAC peptide. In certain embodiments, the constant domain comprises a native or modified TRBC peptide. In certain embodiments, the constant domain is capable of forming a homodimer or a heterodimer with another constant domain. In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a ligand for a cell-surface receptor, a receptor for a cell surface ligand, an antigen binding portion of an antibody or a fragment thereof or an antigen binding portion of a TCR. In certain embodiments, the extracellular antigen-binding domain of the TCR like fusion molecule comprises one or two immunoglobulin variable region(s). In certain embodiments, the extracellular antigen-binding domain of the TCR like fusion molecule comprises a heavy chain variable region (V_{H}) of an antibody. In certain embodiments, the extracellular antigen-binding domain of the TCR like fusion molecule comprises a light chain variable region (V_{L}) of an antibody. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a V_{H} of an antibody, wherein the V_{H} is capable of dimerizing with another extracellular antigen-binding domain comprising a V_{L} of the antibody and form a fragment variable (Fv). In certain embodiments, the extracellular antigen-binding domain of the TCR like fusion molecule comprises a V_{L} of an antibody, wherein the V_{L} is capable of dimerizing with another extracellular antigen-binding domain comprising a V_{H} of the antibody and form a fragment variable (Fv).

The TCR like fusion molecule can bind to a tumor antigen or a pathogen antigen. In certain embodiments, the TCR like fusion molecule binds to a tumor antigen. In certain embodiments, the TCR fusion molecule binds to CD19. In certain embodiments, the TCR like fusion molecule binds to human CD19. In certain embodiments, the TCR fusion molecule is designated as “19-HIT”. In certain embodiments, the TCR like fusion molecule (e.g., 19-HIT) comprises an amino acid sequence that is at least about 85%, about 90%, about 95%, about 96%, about 97%, about 98%, about 99% or about 100% homologous or identical to the amino acid sequence set forth in SEQ ID NO: 111, which is provided below. In certain embodiments, the TCR like fusion molecule (e.g., 19-HIT) comprises the amino acid sequence set forth in SEQ ID NO: 111. The TCR fusion molecule comprising the amino acid sequence set forth in SEQ ID NO: 111 is able to bind to CD19 (e.g., human CD19).

[SEQ ID NO: 111]
MALPVTALLLPLALLLHAEVKLQQSGAELVRPGSSVKISCKASGYAFSSYW
MNWVKQRPGQGLEWIGQIYPGDGDTNYNGKFKGQATLTADKSSSTAYMQLS
GLTSEDSAVYFCARKTISSVVDFYFDYWGQGTTVTVLEDLKNVEPPEVAVF
EPSEAEISHTQKATLVCLATGFYPDHVELSWWVNGKEVHSGVSTDPQPLKE
QPALNDSRYCLSSRLRVSATFWQNPRNHERCQVQFYGLSENDEWTQDRAKP
VTQIVSAEAWGRADCGFTSESYQQGVLSATILYEILLGKATLYAVLVSALV
LMAMVKRKDSRGGSGATNFSLLKQAGDVEENPGPMALPVTALLLPLALLLH
ADIELTQSPKFMSTSVGDRVSVTCKASQNVGTNVAWYQQKPGQSPKPLIYS
ATYRNSGVPDRFTGSGSGTDFTLTITNVQSKDLADYFCQQYNRYPYTSGGG
TKLEIIP

In certain embodiments, the cell comprises a TCR like fusion molecule and a presently disclosed fusion polypeptide. In certain embodiments, the cell expresses a high level of the fusion polypeptide.

### 5.5. Compositions and Vectors

The present discloses subject matter provides compositions comprising a fusion polypeptide disclosed herein (e.g., disclosed in Section 5.2) and an antigen-recognizing receptor disclosed herein (e.g., disclosed in Section 5.4). Also provided are cells comprising such compositions.

In certain embodiments, the fusion polypeptide is operably linked to a first promoter. In certain embodiments, the antigen-recognizing receptor is operably linked to a second promoter.

Furthermore, the present discloses subject matter provides nucleic acid compositions comprising a first polynucleotide encoding a fusion polypeptide disclosed herein (e.g., disclosed in Section 5.2) and a second polynucleotide encoding an antigen-recognizing receptor disclosed herein (e.g., disclosed in Section 5.4). Also provided are cells comprising such nucleic acid compositions.

In certain embodiments, the nucleic acid composition further comprises a first promoter that is operably linked to the fusion polypeptide. In certain embodiments, the nucleic acid composition further comprises a second promoter that is operably linked to the antigen-recognizing receptor.

In certain embodiments, one or both of the first and second promoters are endogenous or exogenous.

In certain embodiments, the exogenous promoter is selected from an elongation factor (EF)-1 promoter, a CMV promoter, a SV40 promoter, a PGK promoter, and a metallothionein promoter. In certain embodiments, one or both of the first and second promoters are inducible promoters. In certain embodiment, the inducible promoter is selected from a NFAT transcriptional response element (TRE) promoter, a CD69 promoter, a CD25 promoter, and an IL-2 promoter.

In certain embodiments, the antigen-recognizing receptor is a TCR, and the fusion polypeptide is operably linked to a first promoter, wherein the first promoter is capable of inducing a stable and high expression level of the fusion polypeptide.

In certain embodiments, the antigen-recognizing receptor is a TCR like fusion molecule (e.g., a HIT CAR), and the fusion polypeptide is operably linked to a first promoter, wherein the first promoter is capable of inducing a stable and high expression level of the fusion polypeptide.

In certain embodiments, the antigen-recognizing receptor is a chimeric antigen receptor and the expression of the chimeric antigen receptor and the fusion polypeptide are under the control of an endogenous promoter. Non-limiting examples of endogenous promoters include an endogenous TRAC promoter, an endogenous TRBC promoter, an endogenous TRDC promoter, and an endogenous TRGC promoter. In certain embodiments, the endogenous promoter is an endogenous TRAC promoter.

The compositions and nucleic acid compositions can be administered to subjects or and/delivered into cells by art-known methods or as described herein. Genetic modification of a cell (e.g., a T cell or a NK cell) can be accomplished by transducing a substantially homogeneous cell composition with a recombinant DNA construct. In certain embodiments, a retroviral vector (either gamma-retroviral or lentiviral) is employed for the introduction of the nucleic acid compositions into the cell. For example, the first polynucleotide encoding the fusion polypeptide and the second polynucleotide encoding the antigen-recognizing receptor can be cloned into a retroviral vector and expression can be driven from its endogenous promoter, from the retroviral long terminal repeat, or from a promoter specific for a target cell type of interest. Non-viral vectors may be used as well.

For initial genetic modification of a cell to include an antigen-recognizing receptor (e.g., a CAR, a TCR, or a TCR like fusion molecule), a retroviral vector is generally employed for transduction, however any other suitable viral vector or non-viral delivery system can be used. The antigen-recognizing receptor and the fusion polypeptide can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art. In certain embodiments, the P2A peptide comprises or has the amino acid sequence set forth in SEQ ID NO: 108, which is provided below:

[SEQ ID NO: 108]
GSGATNFSLLKQAGDVEENPGPM

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to deliver the fusion polypeptide and/or the antigen-recognizing receptor disclosed herein to a cell or a subject. In certain embodiments, a CRISPR system is used to deliver the fusion polypeptide and/or the antigen-recognizing receptor disclosed herein. In certain embodiments, zinc-finger nucleases are used to deliver the fusion polypeptide and/or the antigen-recognizing receptor disclosed herein. In certain embodiments, a TALEN system is used to deliver the fusion polypeptide and/or the antigen-recognizing receptor disclosed herein.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

The composition or nucleic acid composition disclosed herein can be placed anywhere in a genome. In certain embodiments, the composition or nucleic acid composition is placed in a locus within the genome of a T cell, including, but not limited to, a TRAC locus, a TRBC locus, a TRDC locus, and/or a TRGC locus. In certain embodiments, the placement of the composition or nucleic acid composition disrupts the expression of an endogenous T cell receptor. In certain embodiments, the antigen-recognizing receptor is a chimeric antigen receptor (CAR).

In certain embodiments, the composition or nucleic acid composition is integrated at a locus encoding for an immune inhibitory molecule. Non-limiting examples of immune inhibitory molecules include CTLA-4, PD-1, LAG3, BTLA, B7-1, B7-H1, B7-H3, B7-H4, TIM3, SHP-1, SHP-2, TIGIT, CD160, and LAIR1.

### 5.6. Polypeptides and Analogs

Also included in the presently disclosed subject matter are polypeptides disclosed herein (e.g., CD19, 4-1BB, CD80, CD28, CD3ζ, etc. or fragments thereof) that are modified in ways for desired purpose, e.g., for enhancing their anti-neoplastic and/or anti-tumor activity when expressed in a cell. The presently disclosed subject matter provides methods for optimizing an amino acid sequence or nucleic acid sequence by producing an alteration in the sequence, and modified amino acid sequences and nucleic acid sequences. Such alterations may include certain mutations, deletions, insertions, or post-translational modifications. The presently disclosed subject matter further includes analogs of any polypeptide disclosed herein. Analogs can differ from a polypeptide disclosed herein by amino acid sequence differences, by post-translational modifications, or by both. Analogs can exhibit at least about 85%, about 90%, about 91%, about 92%, about 93%, about 94%, about 95%, about 96%, about 97%, about 98%, about 99% or more homologous to all or part of an amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amina acids, e.g., D-amino acids or non-naturally occurring or synthetic amino acids, e.g., β or γ amino acids.

In addition to full-length polypeptides, the presently disclosed subject matter also provides fragments of any one of the polypeptides disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein disclosed herein (e.g., a fusion polypeptide). Such analogs may exceed the physiological activity of the original polypeptide. Methods of analog design are well known in the art, and synthesis of analogs can be carried out according to such methods by modifying the chemical structures such that the resultant analogs increase the anti-neoplastic activity of the original polypeptide when expressed in a cell. These chemical modifications include, but are not limited to, substituting alternative R groups and varying the degree of saturation at specific carbon atoms of a reference polypeptide. In certain embodiments, the protein analogs are relatively resistant to in vivo degradation, resulting in a more prolonged therapeutic effect upon administration. Assays for measuring functional activity include, but are not limited to, those described in the Examples below.

### 5.7. Administration

Compositions comprising the presently disclosed cells can be provided systemically or directly to a subject for inducing and/or enhancing an immune response to an antigen and/or treating and/or preventing a neoplasm (e.g., cancer), pathogen infection, or infectious disease. In certain embodiments, the presently disclosed cells, compositions, or nucleic acid compositions are directly injected into an organ of interest (e.g., an organ affected by a neoplasm). Alternatively, the presently disclosed cells, compositions, or nucleic acid compositions are provided indirectly to the organ of interest, for example, by administration into the circulatory system (e.g., the tumor vasculature). Expansion and differentiation agents can be provided prior to, during or after administration of the cells, compositions, or nucleic acid compositions to increase production of the cells (e.g., T cells (e.g., CTL cells) or NK cells) in vitro or in vivo.

The presently disclosed cells, compositions, or nucleic acid compositions can be administered in any suitable routes, including but not limited to, intravenous, subcutaneous, intranodal, intratumoral, intrathecal, intrapleural, intraperitoneal. Usually, at least about 1×10⁵ cells will be administered, eventually reaching about 1×10¹⁰ or more. The presently disclosed cells can comprise a purified population of cells. Those skilled in the art can readily determine the percentage of the presently disclosed cells in a population using various well-known methods, such as fluorescence activated cell sorting (FACS). Suitable ranges of purity in populations comprising the presently disclosed cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like. The presently disclosed compositions can be pharmaceutical compositions comprising the presently disclosed cells or their progenitors and a pharmaceutically acceptable carrier. Administration can be autologous or heterologous. For example, cells, or progenitors can be obtained from one subject, and administered to the same subject or a different, compatible subject. Peripheral blood derived cells or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed cell), it can be formulated in a unit dosage injectable form (solution, suspension, emulsion).

In certain embodiments, the pharmaceutical composition further comprises a regulator that is capable of regulating or modulating the expression and/or activity of the fusion polypeptide (e.g., one disclosed in Section 5.2.4).

### 5.8. Formulations

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified cells or their progenitors.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

The quantity of cells to be administered will vary for the subject being treated. In certain embodiments, between about 10⁴ and about 10¹⁰, between about 10⁵ and about 10⁹, or between about 10⁴ and about 10⁸ of the presently disclosed cells are administered to a human subject. In certain embodiments, between about 10⁴ and about 10⁷ of the presently disclosed cells are administered to a human subject. More effective cells may be administered in even smaller numbers. In certain embodiments, at least about 1×10⁴ of the presently disclosed cells are administered to a human subject. In certain embodiments, at least about 1×10⁵ of the presently disclosed cells are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### 5.9. Methods of Treatment

The presently disclosed subject matter provides methods for inducing and/or increasing an immune response in a subject in need thereof. The presently disclosed cells, compositions, and nucleic acid compositions can be used in a therapy or medicament. The presently disclosed cells and compositions comprising thereof can be used for treating and/or preventing a neoplasm in a subject. The presently disclosed cells, compositions and nucleic acid compositions can be used for prolonging the survival of a subject suffering from a neoplasm. The presently disclosed cells, compositions, and nucleic acid compositions can also be used for treating and/or preventing a neoplasm in a subject. In certain embodiments, the neoplasm is cancer. The presently disclosed cells, compositions, and nucleic acid compositions can also be used for treating and/or preventing a pathogen infection or other infectious disease in a subject, such as an immunocompromised human subject. The presently disclosed cells, compositions, and nucleic acid compositions can also be used for treating and/or preventing an autoimmune disease in a subject. Such methods comprise administering the presently disclosed cells in an amount effective, a presently disclosed composition (e.g., pharmaceutical composition), or a presently disclosed nucleic acid composition to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

An “effective amount” (or, “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

For adoptive immunotherapy using antigen-specific T cells, cell doses in the range of about 10⁶-10¹⁰ (e.g., about 10⁹) are typically infused. Upon administration of the presently disclosed cells into the host and subsequent differentiation, T cells are induced that are specifically directed against the specific antigen.

The presently disclosed subject matter provides methods for treating and/or preventing a neoplasm in a subject. The method can comprise administering an effective amount of the presently disclosed cells, a presently disclosed composition, or a presently disclosed nucleic acid composition to a subject having a neoplasm.

Non-limiting examples of neoplasms include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolar carcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasm is cancer. In certain embodiments, the neoplasm is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed cells, compositions, nucleic acid compositions can be used for treating and/or preventing blood cancers (e.g., leukemias, lymphomas, and myelomas) or ovarian cancer, which are not amenable to conventional therapeutic interventions. In certain embodiments, the presently disclosed cells, compositions, nucleic acid compositions can be used for treating and/or preventing a solid tumor.

The presently disclosed subject matter provides methods for treating and/or preventing a viral infection in a subject. The method can comprise administering an effective amount of the presently disclosed cells, a presently disclosed composition, or a presently disclosed nucleic acid composition to a subject having a viral infection. Non-limiting examples of viral infections include those caused by cytomegalovirus (CMV), Epstein-Barr virus (EBV), hepatitis A, B, C, D, E, F or G, human immunodeficiency virus (HIV), adenovirus, BK polyomavirus, coronavirus, coxsackievirus, poliovirus, herpes simplex type 1, herpes simplex type 2, human cytomegalovirus, human herpesvirus type 8, varicella-zoster virus, influenza virus, measles virus, mumps virus, parainfluenza virus, respiratory syncytial virus, papillomavirus, rabies virus, and Rubella virus. Other viral targets include Paramyxoviridae (e.g., pneumovirus, morbillivirus, metapneumovirus, respirovirus or rubulavirus), Adenoviridae (e.g., adenovirus), Arenaviridae (e.g., arenavirus such as lymphocytic choriomeningitis virus), Arteriviridae (e.g., porcine respiratory and reproductive syndrome virus or equine arteritis virus), Bunyaviridae (e.g., phlebovirus or hantavirus), Caliciviridae (e.g., Norwalk virus), Coronaviridae (e.g., coronavirus or torovirus), Filoviridae (e.g., Ebola-like viruses), Flaviviridae (e.g., hepacivirus or flavivirus), Herpesviridae (e.g., simplexvirus, varicellovirus, cytomegalovirus, roseolovirus, or lymphocryptovirus), Orthomyxoviridae (e.g., influenza virus or thogotovirus), Parvoviridae (e.g., parvovirus), Picomaviridae (e.g., enterovirus or hepatovirus), Poxviridae (e.g., orthopoxvirus, avipoxvirus, or leporipoxvirus), Retroviridae (e.g., lentivirus or spumavirus), Reoviridae (e.g., rotavirus), Rhabdoviridae (e.g., lyssavirus, novirhabdovirus, or vesiculovirus), and Togaviridae (e.g., alphavirus or rubivirus). In certain embodiments, the viral infections include human respiratory coronavirus, influenza viruses A-C, hepatitis viruses A to G, and herpes simplex viruses 1-9. In certain embodiments, the subject has an immunodeficiency. The presently disclosed subject matter provides methods for treating and/or preventing a bacterial infection in a subject. The method can comprise administering an effective amount of the presently disclosed cells, a presently disclosed composition, or a presently disclosed nucleic acid composition to a subject having a bacterial infection. Bacterial infections include, but are not limited to, Mycobacteria, Rickettsia, Mycoplasma, Neisseria meningitides, Neisseria gonorrheoeae, Legionella, Vibrio cholerae, Streptococci, Staphylococcus aureus, Staphylococcus epidermidis, Pseudomonas aeruginosa, Corynobacteria diphtherias, Clostridium spp., enterotoxigenic Escherichia coli, Bacillus anthracis, Rickettsia, Bartonella henselae, Bartonella quintana, Coxiella burnetii, chlamydia, Mycobacterium leprae, Salmonella, shigella, Yersinia enterocolitica, Yersinia pseudotuberculosis; Legionella pneumophila; Mycobacterium tuberculosis; Listeria monocytogenes; Mycoplasma spp., Pseudomonas fluorescens, Vibrio cholerae, Haemophilus influenzae, Bacillus anthracis, Treponema pallidum, Leptospira, Borrelia, Corynebacterium diphtherias, Francisella, Brucella melitensis, Campylobacter jejuni, Enterobacter, Proteus mirabilis, Proteus, and Klebsiella pneumoniae.

The presently disclosed subject matter provides methods for treating and/or preventing an autoimmune disease in a subject. The method can comprise administering an effective amount of the presently disclosed cells, a presently disclosed composition, or a presently disclosed nucleic acid composition to a subject having an autoimmune disease.

The presently disclosed subject matter provides methods for treating and/or preventing an autoimmune disease in a subject. The method can comprise administering an effective amount of the presently disclosed cells, a presently disclosed composition, or a presently disclosed nucleic acid composition to a subject having an infectious disease.

Non-limiting examples of autoimmune diseases and inflammatory diseases or conditions thereof include arthritis, e.g., rheumatoid arthritis (RA), Type I diabetes, systemic lupus erythematosus (SLE), inflammatory bowel disease, ulcerative colitis, psoriasis, psoriatic arthritis, scleroderma, autoimmune thyroid disease, Grave's disease, Crohn's disease, multiple sclerosis, systemic sclerosis, asthma, organ transplant rejection, a disease or condition associated with transplant, Takayasu arteritis, giant-cell arteritis, Kawasaki disease, polyarteritis nodosa, Behcet's syndrome, Wegener's granulomatosis, ANCA-vasculitides, Churg-Strauss syndrome, microscopic polyangiitis, vasculitis of connective tissue diseases, Hennoch-Schonlein purpura, cryoglobulinemic vasculitis, cutaneous leukocytoclastic angiitis, Sarcoidosis, Cogan's syndrome, Wiskott-Aldrich Syndrome, primary angiitis of the CNS, thromboangiitis obliterans, paraneoplastic arteritis, myelodysplastic syndrome, erythema elevatum diutinum, amyloidosis, autoimmune myositis, Guillain-Barre Syndrome, histiocytosis, atopic dermatitis, pulmonary fibrosis, glomerulonephritis, Whipple's disease, Still's disease, Sjogren's syndrome, osteomyelofibrosis, chronic inflammatory demyelinating polyneuropathy, Kimura's disease, systemic sclerosis, chronic periaortitis, chronic prostatitis, idiopathic pulmonary fibrosis, chronic granulomatous disease, idiopathic, bleomycin-induced lung inflammation, cytarabine-induced lung inflammation, autoimmune thrombocytopenia, autoimmune neutropenia, autoimmune hemolytic anemia, autoimmune lymphocytopenia, chronic autoimmune thyroiditis, autoimmune hepatitis, Hashimoto's thyroiditis, atopic thyroiditis, Graves disease, autoimmune polyglandular syndrome, autoimmune Addison syndrome, and/or myasthenia gravis. In accordance with the presently disclosed subject matter, the above-described various methods can comprise administering to the subject a checkpoint immune blockade agent.

In certain embodiments, the checkpoint immune blockade agent is selected from the group consisting of anti-PD-L1 antibodies, anti-CTLA-4 antibodies, anti-PD-1 antibodies, anti-LAG3 antibodies, anti-B7-H3 antibodies, anti-TIM3 antibodies, and combinations thereof. In certain embodiments, the checkpoint immune blockade agent is an anti-PD-L1 antibody or an anti-PD-1 antibody. In certain embodiments, the checkpoint immune blockade agent is an anti-PD-1 antibody.

In accordance with the presently disclosed subject matter, the above-described various methods can comprise administering to the subject a regulator that is capable of regulating or modulating expression, activity of the fusion polypeptide.

In certain embodiments, the regulator is selected from the group consisting of promoters that are capable of controlling the expression of the fusion polypeptide, molecules that are capable of regulating or modulating expression and/or activity of the co-stimulatory ligand, molecules that are capable of regulating or modulating expression and/or activity of the co-stimulatory molecule.

In certain embodiments, the molecules that are capable of regulating or modulating expression and/or activity of the co-stimulatory ligand are selected from the group consisting of antibodies that bind to the co-stimulatory ligand, and fusion proteins that bind to the co-stimulatory ligand and regulate or modulate the expression and/or activity of the co-stimulatory ligand.

In certain embodiments, the regulator is an anti-CD80 antibody and the fusion polypeptide comprises an extracellular domain and a transmembrane domain of CD80.

In certain embodiments, the regulator is a fusion protein that binds to CD80 and modulates the activity of CD80 and the fusion polypeptide comprises an extracellular domain and a transmembrane domain of CD80. In certain embodiments, the fusion protein is a CTLA-4 fragment that binds to CD80. In certain embodiments, the CTLA-4 fragment that binds to CD80 is Abatacept or belatacept.

In certain embodiments, the molecules that are capable of regulating or modulating expression, activity of the co-stimulatory molecule are selected from the group consisting of antibodies that bind to the co-stimulatory molecule, fusion proteins that bind to the co-stimulatory molecule and regulate or modulate the expression, activity of the co-stimulatory molecule.

In certain embodiments, the regulator is capable of depleting the cell. In certain embodiments, regulator is capable of lessening or eliminating one or more side effects associated with administration of the cell. In certain embodiments, the one or more side effects are selected from the group consisting of off-tumor target effects, cytokine release syndrome, neurotoxicity, and combinations thereof. In certain embodiments, the subject is a human.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition is administered to these subjects to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

A second group of suitable subjects is known in the art as the “adjuvant group.” These are individuals who have had a history of neoplasm, but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasm. Features typical of high-risk subgroups are those in which the tumor has invaded neighboring tissues, or who show involvement of lymph nodes.

Another group have a genetic predisposition to neoplasm but have not yet evidenced clinical signs of neoplasm. For instance, women testing positive for a genetic mutation associated with breast cancer, but still of childbearing age, can wish to receive one or more of the cells described herein in treatment prophylactically to prevent the occurrence of neoplasm until it is suitable to perform preventive surgery.

As a consequence of expression of an antigen-recognizing receptor that binds to a tumor antigen and a fusion polypeptide that enhances the anti-tumor effect of the cell, adoptively transferred cells are endowed with augmented and selective cytolytic activity at the tumor site. Furthermore, subsequent to their localization to tumor or viral infection and their proliferation, the T cells turn the tumor or viral infection site into a highly conductive environment for a wide range of immune cells involved in the physiological anti-tumor or antiviral response (tumor infiltrating lymphocytes, NK−, NKT− cells, dendritic cells, and macrophages).

Additionally, the presently disclosed subject matter provides methods for treating and/or preventing a pathogen infection (e.g., viral infection, bacterial infection, fungal infection, parasite infection, or protozoal infection) in a subject, e.g., in an immunocompromised subject. The method can comprise administering an effective amount of the presently disclosed cells, a presently disclosed composition, or a presently disclosed nucleic acid composition to a subject having a pathogen infection. Exemplary viral infections susceptible to treatment include, but are not limited to, Cytomegalovirus (CMV), Epstein Barr Virus (EBV), Human Immunodeficiency Virus (HIV), and influenza virus infections.

Further modification can be introduced to the presently disclosed cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the upstream of the antigen-recognizing receptor of a presently disclosed CAR. The suicide gene can be included within the vector comprising nucleic acids encoding a presently disclosed CAR. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated CAR-expressing T cells. The incorporation of a suicide gene into the a presently disclosed CAR gives an added level of safety with the ability to eliminate the majority of CAR T cells within a very short time period. A presently disclosed cell (e.g., a T cell) incorporated with a suicide gene can be pre-emptively eliminated at a given timepoint post CAR T cell infusion, or eradicated at the earliest signs of toxicity.

Furthermore, as disclosed in Section 5.2.4, the expression and/or activity of the fusion polypeptide can be regulated or modulated by a regulator (e.g., one disclosed in Section 5.2.4). In certain embodiments, the regulator can deplete the presently disclosed cells, e.g., to lessen or eliminate side effect(s) associated with administration of the presently disclosed cells, e.g., off-tumor target effects, cytokine release syndrome, and/or neurotoxicity.

In certain embodiments, the method or use describe herein further comprises administering a regulator that is capable of regulating or modulating expression and/or activity of the fusion polypeptide (e.g., one disclosed in Section 5.2.4). The regulator can be administered prior to, simultaneously, or post to the administration (e.g., initial administration) of the presently disclosed cells or compositions comprising thereof. In certain embodiments, the method or use describe herein further comprises administering an anti-CD80 antibody, and the fusion polypeptide comprised in the cells comprises an extracellular domain and a transmembrane domain of CD80. In certain embodiments, the method or use describe herein further comprises administering a fusion protein that binds to CD80 and modulates the activity of CD80, and the fusion polypeptide comprised in the cells comprises an extracellular domain and a transmembrane domain of CD80. In certain embodiments, the fusion protein is a CTLA-4 fragment that binds to CD80, e.g.,

### 5.10. Kits

The presently disclosed subject matter provides kits for inducing and/or enhancing an immune response and/or treating and/or preventing a neoplasm or a pathogen infection in a subject. In certain embodiments, the kit comprises an effective amount of presently disclosed cells, a presently disclosed composition, or a presently disclosed nucleic acid composition. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid molecule encoding an antigen-recognizing receptor (e.g., a CAR or a TCR) directed toward an antigen of interest and an isolated nucleic acid molecule encoding a fusion polypeptide in expressible form, which may optionally be comprised in the same or different vectors.

If desired, the cells, composition, or nucleic acid composition are provided together with instructions for administering the cells, composition, or nucleic acid composition to a subject having or at risk of developing a neoplasm (e.g., a cancer) or a pathogen infection (e.g., an infectious disease), or immune disorder (e.g., an autoimmune disease). The instructions generally include information about the use of the cell, composition or nucleic acid composition for the treatment and/or prevention of a neoplasm, or a pathogen infection (e.g., an infectious disease), or an immune disorder (e.g., an autoimmune disease). In certain embodiments, the instructions include at least one of the following: description of the therapeutic agent; dosage schedule and administration for treatment or prevention of a neoplasm, pathogen infection (e.g., an infectious disease), or immune disorder (e.g., an autoimmune disease) or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 6. EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1—CD80/4-1BB Co-Stimulatory Molecule

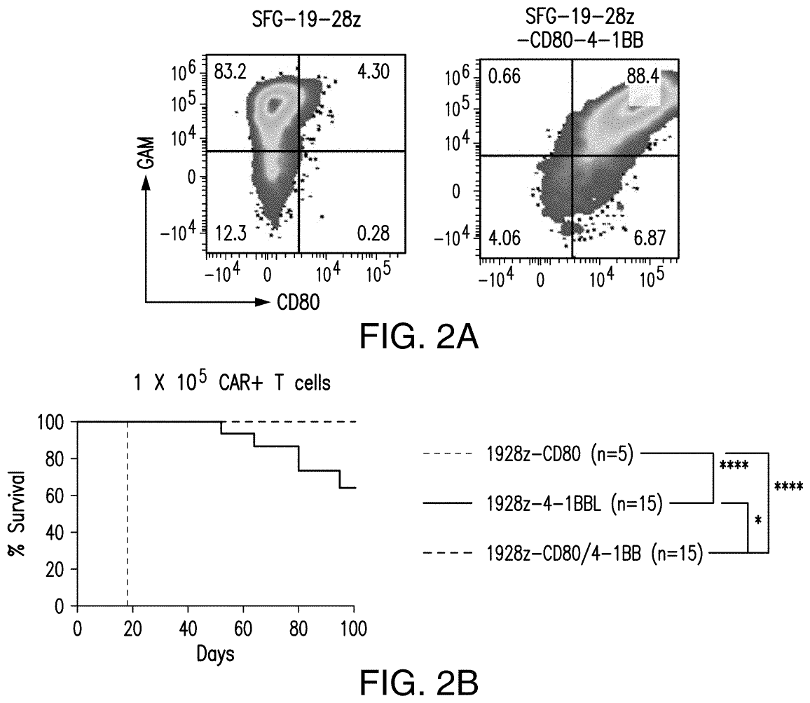

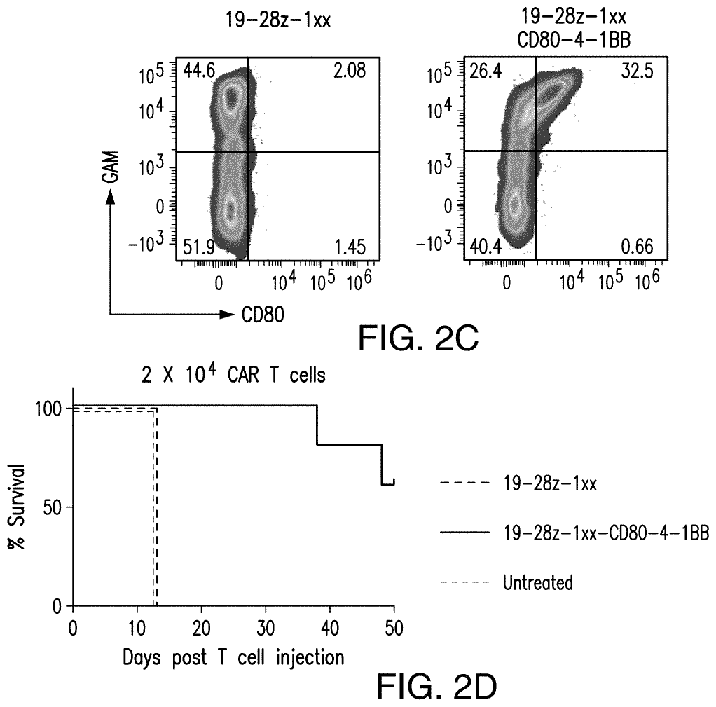

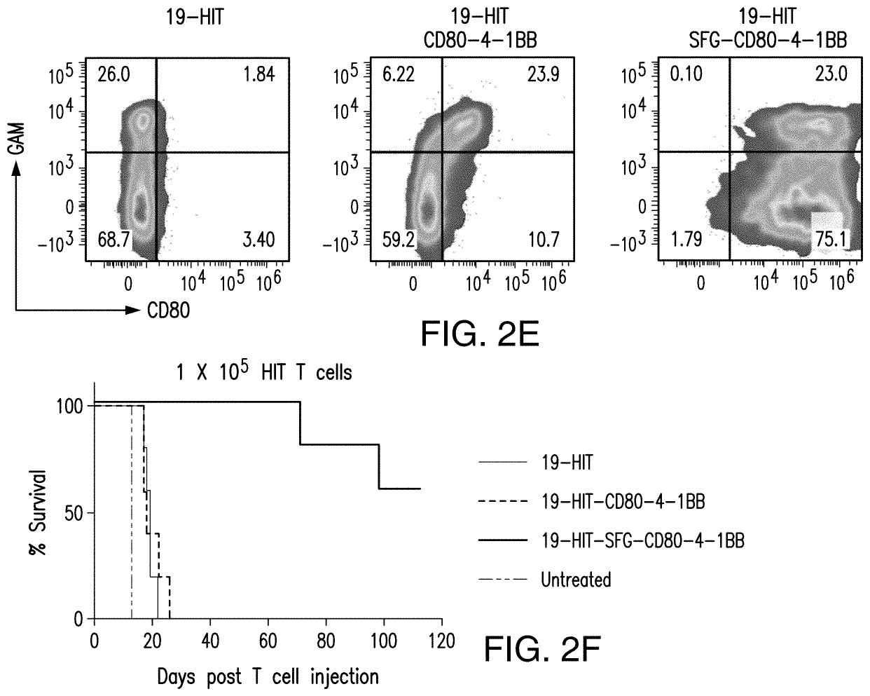

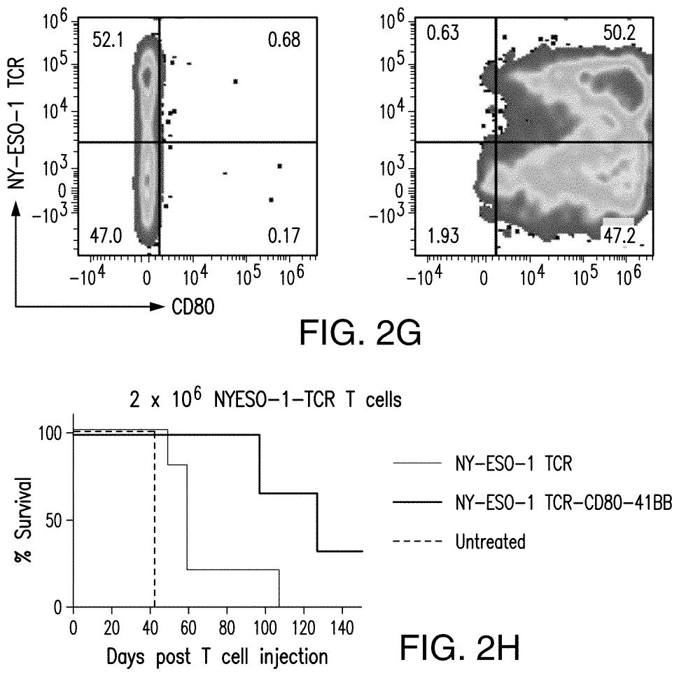

A presently disclosed fusion polypeptide was generated. The fusion polypeptide comprises the extracellular domain and transmembrane domain of human CD80 and the intracellular domain of human 4-1BB (designated as “CD80/4-1BB fusion polypeptide”). This CD80/4-1BB fusion polypeptide was transfected to T cells comprising a CAR targeting human CD19 (“1928z” or “1928z-1xx”), a CD19-targeting TCR like fusion molecule, which is an HLA independent TCR receptor (HIT) (designated as “19-HIT”), or a NY-ESO-1 TCR. The expressions of 1928z CAR with the CD80/4-1BB fusion polypeptide, 1928z CAR without the CD80/4-1BB fusion polypeptide, 1928z-1xx CAR with the CD80/4-1BB fusion polypeptide, 1928z-1xx CAR without the CD80/4-1BB fusion polypeptide, 19-HIT with the CD80/4-1BB fusion polypeptide, 19-HIT without the CD80/4-1BB fusion polypeptide were measured, NY-ESO-1 TCR with the CD80/4-1BB fusion polypeptide, or NY-ESO-1 TCR without the CD80/4-1BB were measured. The FACS expression results are shown in FIGS. 2A, 2C, 2E, and 2G. U.S. Pat. No. 9,220,728 discloses that exogenous co-stimulatory ligands (e.g., CD80, 4-1BBL) can enhance the cytotoxicity of CAR T cells. To compare to the cytotoxicity of CAR T cells comprising an exogenous co-stimulatory ligand, mice bearing NALM6 CD19 leukemia cells were treated with (a) 10⁵ T cells comprising 1928z and this CD80/4-1BB fusion polypeptide (“1928z-CD80/4-1BB”), (b) 10⁵ T cells comprising 1928z and an exogenous 4-1BBL molecule (“1928z-4-1BBL”), or (c) 10⁵ T cells comprising 1928z and an exogenous CD80 molecule (“1928z-CD80”). The survival rate of these mice were measured, and the results are shown in FIG. 2B. As shown in FIG. 2B, the CD80/4-1BB fusion polypeptide enhanced cytotoxicity and/or anti-tumor activity of the T cells (e.g., increased survival rate of the mice treated with T cells expressing 1928z-CD80/4-1BB as compared to mice treated with T cells expressing 1928z-4-1BBL or T cells expressing 1928z-CD80).

Furthermore, NALM6 CD19 leukemia cells were treated with (a) 2×10⁴ T cells comprising 19-28z-1xx and this CD80/4-1BB fusion polypeptide (“19-28z-1xx-CD80/4-1BB”), (b) 2×10⁴ T cells comprising 19-28z-1xx, (c) 10⁵ T cells comprising 19-HIT, or (d) 10⁵ T cells comprising 19-HIT and this CD80/4-1BB fusion polypeptide (“19-HIT-CD80/4-1BB”). The survival rates of these mice were measured and the results are shown in FIGS. 2D and 2F. In addition, mice bearing SK-MEL-23 melanoma cell line expressing HLAA2.1/NYESO-complex were treated with 2×10⁶ NY-ESO-1 TCR T cells without the CD80/4-1BB fusion polypeptide or 10⁶ NY-ESO-1 TCR T cells with the CD80/4-1BB fusion polypeptide. The survival rates of these mice were measured and the results are shown in FIG. 2H.

As shown in FIGS. 2D, 2F, and 2H, the CD80/4-1BB fusion polypeptide improved the survival rates as compared to untreated mice and mice treated with the T cells comprising 19-28z-1xx CAR alone or T cells comprising 19-HIT alone. The survival rate of mice treated with 19-HIT-CD80/4-1BB, wherein the 19-HIT and the CD80/4-1BB fusion polypeptide were targeted into TRAC locus and expressed under the control of the endogenous TRAC promoter, was probably due to the low expression of the CD80/4-1BB fusion polypeptide. All the results shown herein support the broad usage of the presently disclosed CD80/4-1BB fusion polypeptide to improve the antitumor T cell response.

### Example 2

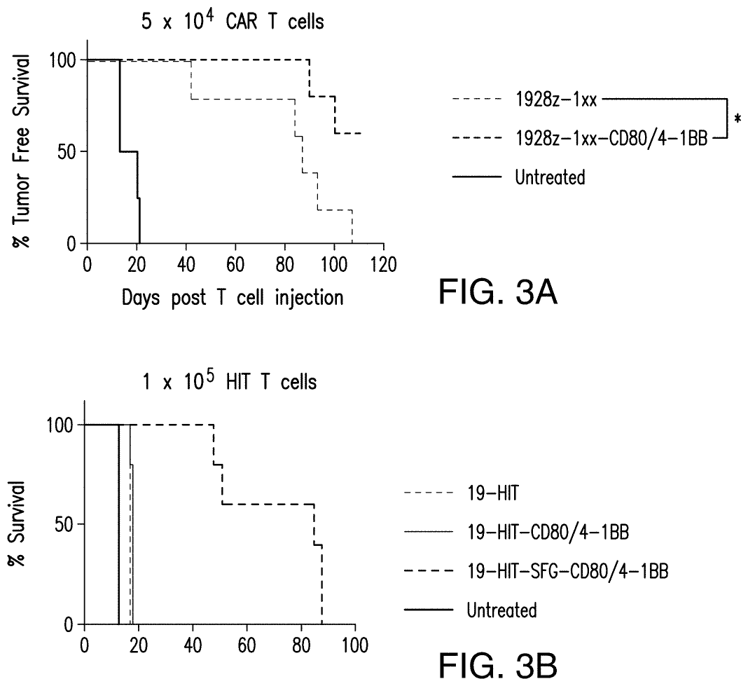

Mice bearing NALM6 CD19 leukemia cells overexpressing PDL-1 molecule were treated with 5×10⁴ T cells comprising 19-28z-1xx without the CD80/4-1BB fusion polypeptide, 5×10⁴ T cells comprising 19-28z-1xx with the CD80/4-1BB fusion polypeptide, 10⁵ T cells comprising 19-HIT without the CD80/4-1BB fusion polypeptide, or 10⁵T cells comprising 19-HIT with the CD80/4-1BB fusion polypeptide. The survival rates of these mice were measured and the results are shown in FIGS. 3A and 3B. As shown in FIGS. 3A and 3B, the presently disclosed CD80/4-1BB fusion polypeptide can preserve T cell antitumor response despite the presence of inhibitory molecules such as PD-1 present in the tumor microenvironment.

### Example 3

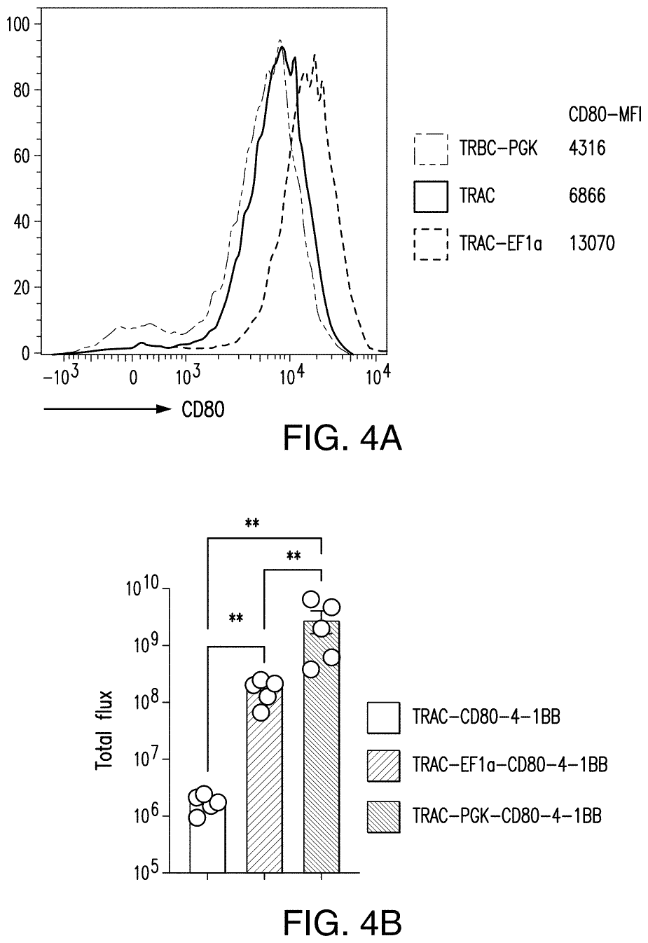

The inventors investigated whether the expression level of the CD80/4-1BB fusion polypeptide governs the anti-tumor response efficiency. The data shown in FIGS. 4A and 4B underscore the fine-tune regulation of the T cell antitumor response determined by the level of expression CD80-41-BB. While HIT and TCR specific T cells benefit from high expression of CD80-4-1BB, CAR T cells confer an improved anti-tumor response when CD80-4-1BB is expressed under the endogenous TRAC promoter.

### Example 4

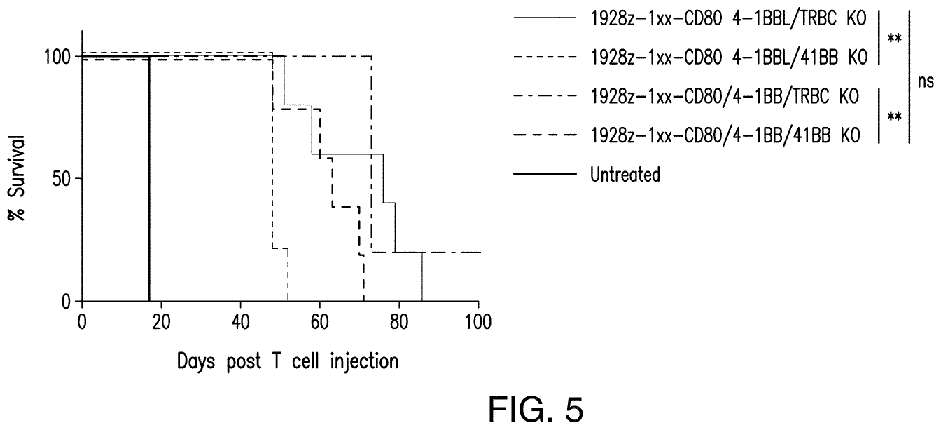

It was investigated whether the presently disclosed CD80/4-1BB fusion polypeptide can compensate for the lack of endogenous 4-1BB. Mice bearing NALM6 CD19 leukemia cells were treated with 2.5×10⁴ T cells comprising 1928z-1xx and an exogenous 4-1BBL molecule and an exogenous CD80 molecule (“1928z-1xx-CD80 4-1BBL”) or 2.5×10⁴ T cells comprising 1928z-1xx and the CD80/4-1BB fusion polypeptide (1928z-1xx-CD80/4-1BB″). Endogenous 4-1BB-gene knockout was obtained by electroporation of protein CAS9 and specific gRNA. Disruption of endogenous 4-1BB was achieved in 68% of infused CAR T cells. TRBC gRNA was used as a control. The survival rates of these mice were measured and the results are shown in FIG. 5. As shown in FIG. 5, despite the combination of exogenous CD80 and exogenous 4-1BBL and the CD80/4-1BB fusion polypeptide offered comparable antitumor effects of CAR T cells with the CD80/4-1BB fusion polypeptide offering better survival, the former (combination of exogenous CD80 and exogenous 4-1BBL) relies on endogenous expression of 4-1BB-molecule upregulated upon T cell activation, in contrast to the CD80/4-1BB fusion polypeptide. In absence of endogenous 4-1BB, the CD80/4-1BB fusion polypeptide improved CAR T cell antitumor response to a level similar to the CD80-4-1BBL TRBC knockout.

### Example 5

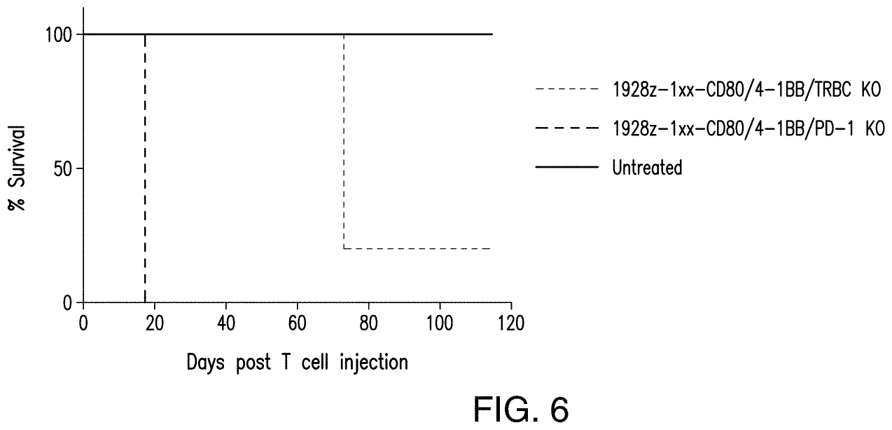

Next, the inventors investigated whether disruption of inhibitory molecule PD-1 enhances antitumor response in presence of CD80-4-1BB. Mice bearing NALM6 CD19 leukemia cells were treated with 2.5×10⁴ T cells comprising 1928z-1xx with the presently disclosed CD80/4-1BB fusion polypeptide. Endogenous PD-1-gene knockout was obtained by electroporation of protein CAS9 and specific gRNA. Disruption of PD-1 was achieved in 55% of infused CAR T cells. TRBC gRNA was used as a control. The survival rates of these mice were measured and the results are shown in FIG. 6. The data shown in FIG. 6 demonstrate the feasibility of gene engineering multiplexing and highlight the benefit of KO PD-1 molecule in the context T cells expressing the presently disclosed CD80/4-1BB fusion polypeptide.

### Example 6

CD4⁺ CAR T cells are known to sustain the CD8⁺ CAR T cells response. However, to mitigate side effects derived from CD4⁺ T cells presence, the inventors investigated whether the presently disclosed CD80/4-1BB fusion polypeptide can compensate for the absence of CD4⁺ CAR T cells.

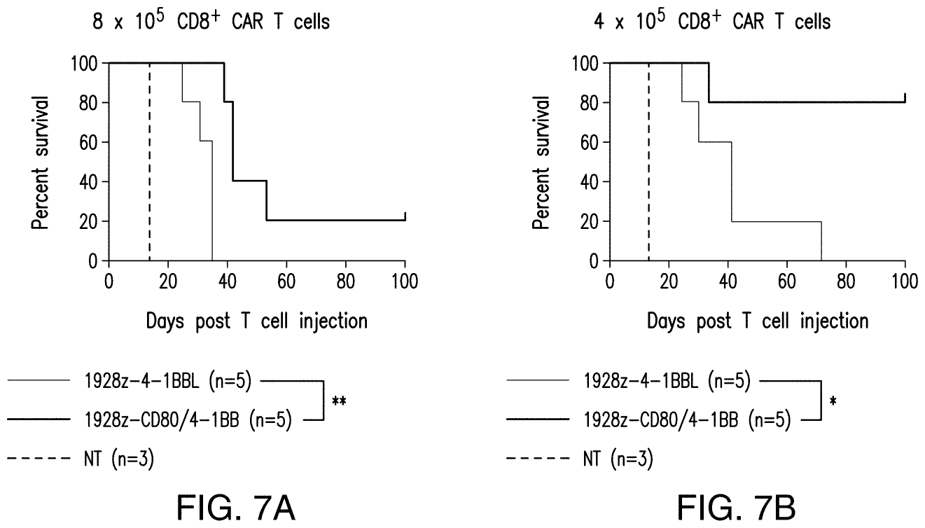

Mice bearing NALM6 CD19 leukemia cells were treated with (a) 4×10⁵ CD8⁺ T cells comprising 1928z and the presently disclosed CD80/4-1BB fusion polypeptide (“1928z-CD80/4-1BB”), (b) 4×10⁵ CD8⁺ T cells comprising 1928z and an exogenous 4-1BBL molecule (“1928z-4-1BBL”), (c) 8×10⁵ CD8⁺ T cells comprising 1928z-CD80/4-1BB, or (d) 8×10⁵ CD8⁺ T cells comprising 1928z-4-1BBL. The survival rates of these mice were measured and the results are shown in FIGS. 7A and 7B. As shown in FIGS. 7A and 7B, the presently disclosed CD80/4-1BB fusion polypeptide compensated for the absence of CD4⁺ T cells by providing co-stimulation need for optimal CD8⁺ CAR T cell response.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A fusion polypeptide comprising:
a) an extracellular domain and a transmembrane domain of a co-stimulatory ligand, and
b) an intracellular domain of a first co-stimulatory molecule.

2. The fusion polypeptide of claim 1, wherein the co-stimulatory ligand is selected from the group consisting of a tumor necrosis factor (TNF) family member, an immunoglobulin (Ig) superfamily member, and combinations thereof.

3. The fusion polypeptide of claim 2, wherein the TNF family member is selected from the group consisting of 4-1BBL, OX40L, CD70, GITRL, CD40L, and combinations thereof.

4. The fusion polypeptide of claim 2, wherein the Ig superfamily member is selected from the group consisting of CD80, CD86, ICOSLG, and combinations thereof.

5. The fusion polypeptide of claim 1, wherein the co-stimulatory ligand is CD80.

6. The fusion polypeptide of claim 5, wherein the extracellular domain of CD80 comprises an amino acid sequence that is at least about 85% homologous or identical to the amino acid sequence set forth in SEQ ID NO: 1, optionally wherein the extracellular domain of CD80 comprises or consists of the amino acid sequence set forth in SEQ ID NO: 1 or a functional fragment thereof.

7. The fusion polypeptide of claim 5, wherein the transmembrane domain of CD80 comprises an amino acid sequence that is at least about 85% homologous or identical to the amino acid sequence set forth in SEQ ID NO: 2, optionally wherein the transmembrane domain of CD80 comprises or consists of the amino acid sequence set forth in SEQ ID NO: 2 or a fragment thereof.

8. The fusion polypeptide of claim 1, wherein the first co-stimulatory molecule is selected from the group consisting of CD28, 4-1BB, OX40, ICOS, DAP-10, CD27, CD40, NKG2D, CD2, and combinations thereof.

9. The fusion polypeptide of claim 1, wherein the first co-stimulatory molecule is 4-1BB.

10. The fusion polypeptide of claim 9, wherein the intracellular domain of 4-1BB comprises an amino acid sequence that is at least about 85% homologous or identical to the amino acid sequence set forth in SEQ ID NO: 3, optionally wherein the intracellular domain of 4-1BB comprises or consists of the amino acid sequence set forth in SEQ ID NO: 3 or a functional fragment thereof.

11. The fusion polypeptide of claim 1, wherein the co-stimulatory ligand is CD80 and the first co-stimulatory molecule is selected from the group consisting of CD28, 4-1BB, OX40, ICOS, DAP-10, CD27, CD40, NKG2D, CD2, and combinations thereof.

12. The fusion polypeptide of claim 11, wherein the co-stimulatory ligand is CD80 and the first co-stimulatory molecule is 4-1BB.

13. The fusion polypeptide of claim 12, wherein the fusion polypeptide comprises an amino acid sequence that is at least about 85% homologous or identical to the amino acid sequence set forth in SEQ ID NO: 4, optionally wherein the fusion polypeptide comprises or consists of the amino acid sequence set forth in SEQ ID NO: 4.

14. The fusion polypeptide of claim 1, further comprising an intracellular domain of a second co-stimulatory molecule.

15. The fusion polypeptide of claim 14, wherein the second co-stimulatory molecule is selected from the group consisting of CD28, 4-1BB, OX40, ICOS, DAP-10, CD27, CD40, NKG2D, CD2, and combinations thereof.

16. The fusion polypeptide of claim 14, wherein the second co-stimulatory molecule is CD28.

17. The fusion polypeptide of claim 16, wherein the intracellular domain of CD28 comprises an amino acid sequence that is at least about 85% homologous or identical to the amino acid sequence set forth in SEQ ID NO: 5, optionally wherein the intracellular domain of CD28 comprises or consists of the amino acid sequence set forth in SEQ ID NO: 5 or a functional fragment thereof.

18. The fusion polypeptide of claim 14, wherein the co-stimulatory ligand is CD80, the first co-stimulatory molecule is 4-1BB, and the second co-stimulatory molecule is CD28.

19. The fusion polypeptide of claim 18, wherein the fusion polypeptide comprises an amino acid sequence that is at least about 85% homologous or identical to the amino acid sequence set forth in SEQ ID NO: 6, optionally wherein the fusion polypeptide comprises or consists of the amino acid sequence set forth in SEQ ID NO: 6.

20. The fusion polypeptide of claim 1, wherein the fusion polypeptide is capable of a) stimulating a cell comprising an antigen-recognizing receptor, and/or b) enhancing the activity of an immunoresponsive cell comprising an antigen-recognizing receptor, optionally wherein the activity comprises cytotoxicity, cell proliferation, and cell persistence.

21. The fusion polypeptide of claim 20, wherein the antigen-recognizing receptor is a chimeric antigen receptor (CAR), a T-Cell Receptor (TCR), or a TCR like fusion molecule.

22. The fusion polypeptide of claim 1, further comprising a signaling domain of a cytokine receptor.

23. The fusion polypeptide of claim 22, wherein the cytokine receptor is selected from the group consisting of CD121a, CDw121b, IL-18Ra, IL18Rb, CD122, CD25, CD132, CD124, CD213a13, CD127, IL-9R, IL15Ra, CDw125, CDw131, CD126, CD130, IL11Ra, Cd114, CD212, CD4, CDw217, CD118, and CDw119.

24. A nucleic acid encoding a fusion polypeptide of claim 1.

25. A vector comprising the nucleic acid of claim 24.

26. A cell comprising a fusion polypeptide of claim 1.

27. A composition comprising an effective amount of the cells of claim 26.

28. A composition comprising: a) a fusion polypeptide of claim 1; and b) an antigen-recognizing receptor that binds to an antigen.

29. A nucleic acid composition comprising: a) a first polynucleotide encoding a fusion polypeptide of claim 1; and b) a second polynucleotide encoding an antigen-recognizing receptor that binds to an antigen.

30. A cell comprising the composition of claim 28.

31. A method for producing a cell, the method comprising introducing into a cell a nucleic acid of claim 24.

32. A kit comprising a nucleic acid of claim 24.

33. A method of reducing tumor burden in a subject; treating a subject having a relapse of a neoplasm; treating and/or preventing a neoplasm in a subject; and/or treating and/or preventing an autoimmune disease or an infectious disease in a subject, the method comprising administering to the subject an effective amount of the cells of claim 30.


SEQUENCE LISTING (111 sequences)

SEQ ID NO: 1 - protein, 242 aa, Artificial Sequence
  Synthetic polypeptide
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 2 - protein, 21 aa, Artificial Sequence
  Synthetic polypeptide
LLPSWAITLISVNGIFVICCL

SEQ ID NO: 3 - protein, 42 aa, Artificial Sequence
  Synthetic polypeptide
KRGRKKLLYIFKQPFMRPVQTTQEEDGCSCRFPEEEEGGCEL

SEQ ID NO: 4 - protein, 309 aa, Artificial Sequence
  Synthetic polynucleotide
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFKRGRKKLLYIFKQPFMRPVQTTQEEDGCSCRFPEEEEGGCEL

SEQ ID NO: 5 - protein, 40 aa, Artificial Sequence
  Synthetic polypeptide
RSKRSRLLHSDYMNMTPRRPGPTRKHYQPYAPPRDFAAYR

SEQ ID NO: 6 - protein, 349 aa, Artificial Sequence
  Synthetic polypeptide
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFRSKRSRLLHSDYMNMTPRRPGPTRKHYQPYAPPRDFAAYRKRGRKKLLYIFKQPFMRPVQTTQEEDGCSCRFPEEEEGGCEL

SEQ ID NO: 7 - protein, 15 aa, Artificial Sequence
  Synthetic polypeptide
GGGGSGGGGSGGGGS

SEQ ID NO: 8 - protein, 20 aa, Artificial Sequence
  Synthetic polypeptide
MYRMQLLSCIALSLALVTNS

SEQ ID NO: 9 - protein, 20 aa, Artificial Sequence
  Synthetic polypeptide
MYSMQLASCVTLTLVLLVNS

SEQ ID NO: 10 - protein, 20 aa, Artificial Sequence
  Synthetic polypeptide
METPAQLLFLLLLWLPDTTG

SEQ ID NO: 11 - protein, 20 aa, Artificial Sequence
  Synthetic polypeptide
METDTLLLWVLLLWVPGSTG

SEQ ID NO: 12 - protein, 21 aa, Artificial Sequence
  Synthetic polypeptide
MALPVTALLLPLALLLHAARP

SEQ ID NO: 13 - protein, 18 aa, Artificial Sequence
  Synthetic polypeptide
MALPVTALLLPLALLLHA

SEQ ID NO: 14 - protein, 16 aa, Artificial Sequence
  Synthetic polypeptide
MKWVTFISLLFSSAYS

SEQ ID NO: 15 - protein, 30 aa, Artificial Sequence
  Synthetic polypeptide
MDSKGSSQKGSRLLLLLVVSNLLLCQGVVS

SEQ ID NO: 16 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 17 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 18 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 19 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 20 - protein, 329 aa, Homo sapiens
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 21 - protein, 199 aa, Homo sapiens
MTLHPSPITCEFLFSTALISPKMCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 22 - protein, 302 aa, Homo sapiens
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 23 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 24 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 25 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 26 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 27 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 28 - protein, 92 aa, Homo sapiens
MIHLGHILFLLLLPVAAAQTTPGERSSLPAFYPGTSGSCSGCGSLSLPLLAGLVAADAVASLLIVGAVFLCARPRRSPAQDGKVYINMPGRG

SEQ ID NO: 29 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCRYSCPREEEGSTIPIQEDYRKPEPACSP

SEQ ID NO: 30 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 31 - protein, 216 aa, Homo sapiens
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 32 - protein, 377 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPGGSILGQSNGLSAWTPPSHPTSLPFAEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 33 - protein, 269 aa, Mus musculus
RPQKSLLVEVEEGGNVVLPCLPDSSPVSSEKLAWYRGNQSTPFLELSPGSPGLGLHVGSLGILLVIVNVSDHMGGFYLCQKRPPFKDIWQPAWTVNVEDSGEMFRWNASDVRDLDCDLRNRSSGSHRSTSGSQLYVWAKDHPKVWGTKPVCAPRGSSLNQSLINQDLTVAPGSTLWLSCGVPPVPVAKGSISWTHVHPRRPNVSLLSLSLGGEHPVREMWVWGSLLLLPQATALDEGTYYCLRGNLTIERHVKVIARSAVWLWLLRTGG

SEQ ID NO: 34 - protein, 272 aa, Homo sapiens
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWK

SEQ ID NO: 35 - protein, 263 aa, Artificial Sequence
  Synthetic polypeptide
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 36 - DNA, 789 bp, Artificial Sequence
  Synthetic polynucleotide
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgg

SEQ ID NO: 37 - protein, 6 aa, Artificial Sequence
  Synthetic polypeptide
GYAFSS

SEQ ID NO: 38 - protein, 6 aa, Artificial Sequence
  Synthetic polypeptide
YPGDGD

SEQ ID NO: 39 - protein, 9 aa, Artificial Sequence
  Synthetic polypeptide
KTISSVVDF

SEQ ID NO: 40 - protein, 11 aa, Artificial Sequence
  Synthetic polypeptide
KASQNVGTNVA

SEQ ID NO: 41 - protein, 6 aa, Artificial Sequence
  Synthetic polypeptide
SATYRN

SEQ ID NO: 42 - protein, 9 aa, Artificial Sequence
  Synthetic polypeptide
QQYNRYPYT

SEQ ID NO: 43 - protein, 122 aa, Artificial Sequence
  Synthetic polypeptide
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 44 - protein, 108 aa, Artificial Sequence
  Synthetic polypeptide
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 45 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 46 - protein, 247 aa, Homo sapiens
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 47 - protein, 69 aa, Artificial Sequence
  Synthetic polypeptide
STTTKPVLRTPSPVHPTGTSQPQRPEDCRPRGSVKGTGLDFACDIYIWAPLAGICVALLLSLIITLICY

SEQ ID NO: 48 - protein, 207 aa, Artificial Sequence
  Synthetic polypeptide
TCTACTACTACCAAGCCAGTGCTGCGAACTCCCTCACCTGTGCACCCTACCGGGACATCTCAGCCCCAGAGACCAGAAGATTGTCGGCCCCGTGGCTCAGTGAAGGGGACCGGATTGGACTTCGCCTGTGATATTTACATCTGGGCACCCTTGGCCGGAATCTGCGTGGCCCTTCTGCTGTCCTTGATCATCACTCTCATCTGCTAC

SEQ ID NO: 49 - protein, 81 aa, Artificial Sequence
  Synthetic polypeptide
TTTTGGGTGCTGGTGGTGGTTGGTGGAGTCCTGGCTTGCTATAGCTTGCTAGTAACAGTGGCCTTTATTATTTTCTGGGTG

SEQ ID NO: 50 - protein, 345 aa, Homo sapiens
MAQHHLWILLLCLQTWPEAAGKDSEIFTVNGILGESVTFPVNIQEPRQVKIIAWTSKTSVAYVTPGDSETAPVVTVTHRNYYERIHALGPNYNLVISDLRMEDAGDYKADINTQADPYTTTKRYNLQIYRRLGKPKITQSLMASVNSTCNVTLTCSVEKEEKNVTYNWSPLGEEGNVLQIFQTPEDQELTYTCTAQNPVSNNSDSISARQLCADIAMGFRTHHTGLLSVLAMFFLLVLILSSVFLFRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVI

SEQ ID NO: 51 - DNA, 75 bp, Artificial Sequence
  Synthetic polynucleotide
ttgctgagcgtgctggctatgttctttctgcttgttctcattctgtcttcagtgtttttgttccgtttgttcaag

SEQ ID NO: 52 - protein, 583 aa, Homo sapiens
MESKGASSCRLLFCLLISATVFRPGLGWYTVNSAYGDTIIIPCRLDVPQNLMFGKWKYEKPDGSPVFIAFRSSTKKSVQYDDVPEYKDRLNLSENYTLSISNARISDEKRFVCMLVTEDNVFEAPTIVKVFKQPSKPEIVSKALFLETEQLKKLGDCISEDSYPDGNITWYRNGKVLHPLEGAVVIIFKKEMDPVTQLYTMTSTLEYKTTKADIQMPFTCSVTYYGPSGQKTIHSEQAVFDIYYPTEQVTIQVLPPKNAIKEGDNITLKCLGNGNPPPEEFLFYLPGQPEGIRSSNTYTLTDVRRNATGDYKCSLIDKKSMIASTAITVHYLDLSLNPSGEVTRQIGDALPVSCTISASRNATVVWMKDNIRLRSSPSFSSLHYQDAGNYVCETALQEVEGLKKRESLTLIVEGKPQIKMTKKTDPSGLSKTIICHVEGFPKPAIQWTITGSGSVINQTEESPYINGRYYSKIIISPEENVTLTCTAENQLERTVNSLNVSAISIPEHDEADEISDENREKVNDQAKLIVGIVVGLLLAALVAGVVYWLYMKKSKTASKHVNKDLGNMEENKKLEENNHKTEA

SEQ ID NO: 53 - DNA, 78 bp, Artificial Sequence
  Synthetic polynucleotide
ctaattgtgggaatcgttgttggtctcctccttgctgcccttgttgctggtgtcgtctactggctgtacatgaagaag

SEQ ID NO: 54 - protein, 235 aa, Homo sapiens; identical to SEQ ID NO: 45

SEQ ID NO: 55 - DNA, 75 bp, Artificial Sequence
  Synthetic polynucleotide
atctacatctgggcgcccttggccgggacttgtggggtccttctcctgtcactggttatcaccctttactgcaac

SEQ ID NO: 56 - protein, 221 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQLRLHPLEKCSRMDY

SEQ ID NO: 57 - DNA, 75 bp, Artificial Sequence
  Synthetic polynucleotide
atcacccttggcctgctggtggctggcgtcctggttctgctggtttccctgggagtggccatccacctgtgctgc

SEQ ID NO: 58 - DNA, 75 bp, Artificial Sequence
  Synthetic polynucleotide
ttctggttacccataggatgtgcagcctttgttgtagtctgcattttgggatgcatacttatttgttggcttaca

SEQ ID NO: 59 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 60 - DNA, 75 bp, Artificial Sequence
  Synthetic polynucleotide
ttcctcctctggatccttgcagcagttagttcggggttgtttttttatagctttctcctcacagctgtttctttg

SEQ ID NO: 61 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 62 - DNA, 81 bp, Artificial Sequence
  Synthetic polynucleotide
attgtcatcatcactgtggtagcagccgcagtcataatgggcactgcaggcctcagcacgtacctctataaccgccagcgg

SEQ ID NO: 63 - protein, 39 aa, Artificial Sequence
  Synthetic polypeptide
IEVMYPPPYLDNEKSNGTIIHVKGKHLCPSPLFPGPSKP

SEQ ID NO: 64 - DNA, 117 bp, Artificial Sequence
  Synthetic polynucleotide
caagagctgacttacacgtgtacagcccagaaccctgtcagcaacaattctgactccatctctgcccggcagctctgtgcagacatcgcaatgggcttccgtactcaccacaccggg

SEQ ID NO: 65 - DNA, 116 bp, Artificial Sequence
  Synthetic polynucleotide
accaactggagagaacagtaaactccttgaatgtctctgctataagtattccagaacacgatgaggcagacgagataagtgatgaaaacagagaaaaggtgaatgaccaggcaaaa

SEQ ID NO: 66 - protein, 39 aa, Artificial Sequence
  Synthetic polypeptide
NQLERTVNSLNVPAISIPEHDEADEISDENREKVNDQAK

SEQ ID NO: 67 - protein, 42 aa, Artificial Sequence
  Synthetic polypeptide
AAANQLERTVNSLNVSAISIPEHDEADEISDENREKVNDQAK

SEQ ID NO: 68 - protein, 48 aa, Artificial Sequence
  Synthetic polypeptide
PEHDEADEISDENREKVNDQAKLIVGIVVGLLLAALVAGVVYWLYMKK

SEQ ID NO: 69 - protein, 37 aa, Artificial Sequence
  Synthetic polypeptide
ENREKVNDQAKLIVGIVVGLLLAALVAGVVYWLYMKK

SEQ ID NO: 70 - protein, 65 aa, Artificial Sequence
  Synthetic polypeptide
NQLERTVNSLNVPAISIPEHDEADEISDENREKVNDQAKLIVGIVVGLLLAALVAGVVYWLYMKK

SEQ ID NO: 71 - protein, 66 aa, Artificial Sequence
  Synthetic polypeptide
TCTAENQLERTVNSLNVSAISIPEHDEADEISDENREKVNDQAKLIVGIVVGLLLAALVAGVVYWL

SEQ ID NO: 72 - protein, 44 aa, Artificial Sequence
  Synthetic polypeptide
PEHDEADEISDENREKVNDQAKLIVGIVVGLLLAALVAGVVYWL

SEQ ID NO: 73 - protein, 61 aa, Artificial Sequence
  Synthetic polypeptide
NQLERTVNSLNVSAISIPEHDEADEISDENREKVNDQAKLIVGIVVGLLLAALVAGVVYWL

SEQ ID NO: 74 - protein, 68 aa, Artificial Sequence
  Synthetic polypeptide
AAANQLERTVNSLNVSAISIPEHDEADEISDENREKVNDQAKLIVGIVVGLLLAALVAGVVYWLYMKK

SEQ ID NO: 75 - DNA, 138 bp, Artificial Sequence
  Synthetic polynucleotide
cccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 76 - DNA, 117 bp, Artificial Sequence
  Synthetic polynucleotide
ctgagtgtggttgatttccttcccaccactgcccagcccaccaagaagtccaccctcaagaagagagtgtgccggttacccaggccagagacccagaagggcccactttgtagcccc

SEQ ID NO: 77 - DNA, 117 bp, Artificial Sequence
  Synthetic polynucleotide
tctcatgccaactattacttctgcaacctatcaatttttgatcctcctccttttaaagtaactcttacaggaggatatttgcatatttatgaatcacaactttgttgccagctgaag

SEQ ID NO: 78 - DNA, 117 bp, Artificial Sequence
  Synthetic polynucleotide
gacacgggactctacatctgcaaggtggagctcatgtacccaccgccatactacctgggcataggcaacggaacccagatttatgtaattgatccagaaccgtgcccagattctgac

SEQ ID NO: 79 - DNA, 117 bp, Artificial Sequence
  Synhetic polynucleotide
ggggaatcagtgactgtcactcgagatcttgagggcacctacctctgtcgggccaggagcactcaaggggaggtcacccgcaaggtgaccgtgaatgtgctctccccccggtatgag

SEQ ID NO: 80 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 81 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - DNA, 336 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 83 - protein, 112 aa, Artificial Sequence
  Synthetic polypeptide
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 84 - DNA, 336 bp, Artificial Sequence
  Synthetic polynucleotide
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 85 - protein, 22 aa, Artificial Sequence
  Synthetic polypeptide
QNQLYNELNLGRREEYDVLDKR

SEQ ID NO: 86 - DNA, 66 bp, Artificial Sequence
  Synthetic polynucleotide
cagaaccagctctataacgagctcaatctaggacgaagagaggagtacgatgttttggacaagaga

SEQ ID NO: 87 - protein, 22 aa, Artificial Sequence
  Synthetic polypeptide
QNQLFNELNLGRREEFDVLDKR

SEQ ID NO: 88 - DNA, 66 bp, Artificial Sequence
  Synthetic polynucleotide
cagaaccagctctttaacgagctcaatctaggacgaagagaggagttcgatgttttggacaagaga

SEQ ID NO: 89 - protein, 23 aa, Artificial Sequence
  Synthetic polypeptide
QEGLYNELQKDKMAEAYSEIGMK

SEQ ID NO: 90 - DNA, 69 bp, Artificial Sequence
  Synthetic polynucleotide
caggaaggcctgtacaatgaactgcagaaagataagatggcggaggcctacagtgagattgggatgaaa

SEQ ID NO: 91 - protein, 23 aa, Artificial Sequence
  Synthetic polypeptide
QEGLFNELQKDKMAEAFSEIGMK

SEQ ID NO: 92 - DNA, 69 bp, Artificial Sequence
  Synthetic polynucleotide
caggaaggcctgttcaatgaactgcagaaagataagatggcggaggccttcagtgagattgggatgaaa

SEQ ID NO: 93 - protein, 22 aa, Artificial Sequence
  Synthetic polypeptide
HDGLYQGLSTATKDTYDALHMQ

SEQ ID NO: 94 - DNA, 66 bp, Artificial Sequence
  Synthetic polynucleotide
cacgatggcctttaccagggtctcagtacagccaccaaggacacctacgacgcccttcacatgcag

SEQ ID NO: 95 - protein, 22 aa, Artificial Sequence
  Synthetic polypeptide
HDGLFQGLSTATKDTFDALHMQ

SEQ ID NO: 96 - DNA, 66 bp, Artificial Sequence
  Synthetic polynucleotide
cacgatggccttttccaggggctcagtacagccaccaaggacaccttcgacgcccttcacatgcag

SEQ ID NO: 97 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 98 - DNA, 123 bp, Artificial Sequence
  Synthetic polynucleotide
aatagtagaaggaacagactccttcaaagtgactacatgaacatgactccccggaggcctgggctcactcgaaagccttaccagccctacgcccctgccagagactttgcagcgtaccgcccc

SEQ ID NO: 99 - protein, 41 aa, Artificial Sequence
  Synthetic polynucleotide
NSRRNRLLQSDYMNMTPRRPGLTRKPYQPYAPARDFAAYRP

SEQ ID NO: 100 - DNA, 123 bp, Artificial Sequence; identical to SEQ ID NO: 98
  Synthetic polynucleotide

SEQ ID NO: 101 - protein, 41 aa, Artificial Sequence
  Synthetic polypeptide
RSKRSRLLHSDYMNMTPRRPGPTRKHYQPYAPPRDFAAYRS

SEQ ID NO: 102 - DNA, 123 bp, Artificial Sequence
  Synthetic polynucleotide
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 103 - protein, 41 aa, Artificial Sequence
  Synthetic polypeptide
RSKRSRLLHSDYMNMTPRRPGPTRKHYQPYAPPRDFAAYRK

SEQ ID NO: 104 - DNA, 126 bp, Artificial Sequence
  Synthetic polynucleotide
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 105 - protein, 467 aa, Artificial Sequence
  Synthetic polypeptide
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - DNA, 1401 bp, Artificial Sequence
  Synthetic polynucleotide
gaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 107 - DNA, 54 bp, Artificial Sequence
  Synthetic polynucleotide
atggctctcccagtgactgccctactgcttcccctagcgcttctcctgcatgca

SEQ ID NO: 108 - protein, 23 aa, Artificial Sequence
  Synthetic polynucleotide
GSGATNFSLLKQAGDVEENPGPM

SEQ ID NO: 109 - protein, 467 aa, Artificial Sequence
  Synthetic polypeptide
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 110 - DNA, 1404 bp, Artificial Sequence
  Synthetic polynucleotide
gaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgctaa

SEQ ID NO: 111 - protein, 466 aa, Artificial Sequence
  Synthetic polypeptide
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMALPVTALLLPLALLLHADIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIIP